(19) United States
(12) Patent Application Publication
(10) Pub. No.: US 2017/0274048 A1
(43) Pub. Date: Sep. 28, 2017

(54) METHODS AND COMPOSITIONS FOR MODULATING THE IMUNE SYSTEM

(71) Applicant: BUCK INSTITUTE FOR RESEARCH ON AGING, Novato, CA (US)

(72) Inventors: Joana NEVES, Novato, CA (US); Deepak LAMBA, Novato, CA (US); Heinrich JASPER, Novato, CA (US)

(21) Appl. No.: 15/300,229

(22) PCT Filed: Mar. 27, 2015
(86) PCT No.: PCT/US2015/023141

§ 371 Date: Sep. 28, 2016

Related U.S. Application Data
(60) Provisional application No. 61/972,206, filed on Mar. 28, 2014.

(51) Int. Cl. A61K 38/18; A61K 39/39; A61K 38/17; C12N 5/079; A61K 35/12

(52) U.S. Cl. CPC A61K 38/18; C12N 5/0618; A61K 35/12; A61K 38/17; A61K 39/39; C12N 5/0602

(57) ABSTRACT

Methods and compositions for cell therapy using immune modulating peptides comprising a manf family protein, or fragment thereof, as an adjuvant to improve replacement cell viability and integration. The methods and compositions can be used in the treatment of a variety of diseases and disorders, including retinal diseases.

### CROSS-REFERENCE

This application claims the benefit of U.S. Provisional Application No. 61/972,206, filed Mar. 28, 2014, which application is incorporated herein by reference in its entirety.

### STATEMENT AS TO FEDERALLY SPONSORED RESEARCH

This invention was made with government support under grant EY018177 awarded by the National Institutes of Health. The government has certain rights in the invention.

### BACKGROUND OF THE INVENTION

Cell therapy can be any therapy in which cellular material is injected into a subject. Cell therapy can be targeted at many clinical indications in multiple organs and by several modes of cell delivery. Cell therapy has met with numerous challenges. For example, aged and damaged (or injured) tissues often provide a poor environment for integration (such as inflammatory microenvironments), thus reducing the efficiency of cell replacement therapy. In addition, cell viability represents a challenge for both replacement cells and cells secreting pro-regeneration factors in immunomodulation cell therapy. There is a need in the art to develop compositions and methods that improve cell therapy and/or reducing inflammatory microenvironments that injured tissues can be treated effectively.

### SUMMARY OF THE INVENTION

In a first aspect, disclosed herein are methods of cell therapy comprising: (a) administering an effective amount of an immune modulating peptide comprising a MANF family protein, or fragment thereof, to a subject in need thereof; (b) administering an effective amount of replacement cells to the subject.

In the methods of the first aspect, the immune modulating peptide can be administered prior to the replacement cells. Alternatively, the immune modulating peptide can be administered concurrently with the replacement cells.

In the methods of the first aspect, the replacement cells can be contacted with the immune modulating peptide prior to administration.

In a second aspect, disclosed herein are methods of cell therapy comprising: (a) administering an effective amount of genetically engineered cells comprising a transgene that expresses an immune modulating peptide comprising a MANF family protein, or fragment thereof, to a subject in need thereof; and (b) administering an effective amount of replacement cells to the subject.

In the methods of the second aspect, the genetically engineered cells can be administered prior to the replacement cells. Alternatively, the genetically engineered cells can be administered concurrently with the replacement cells.

In the methods of the second aspect, the replacement cells can be contacted with the immune modulating peptide prior to administration.

In the methods of the second aspect, the genetically engineered cells can be genetically engineered fibroblasts.

In the methods of the first and second aspects, the MANF family protein can be mesencephalic astrocyte-derived neurotrophic factor (MANF) or a fragment thereof.

In the methods of the first and second aspects, the peptide sequence of the immune modulating peptide can comprise a sequence that has at least about 80% identity with SEQ ID NO:3. For example, the peptide sequence of the immune modulating peptide can comprise a sequence that has at least about 90% identity with SEQ ID NO:3. In another example, the peptide sequence of the immune modulating peptide can comprise a sequence that has at least about 95% identity with SEQ ID NO:3. The immune modulating peptide can have a length that is at least 80% the length of SEQ ID NO:3. For example, the immune modulating peptide can have a length that is 100% the length of SEQ ID NO:3.

In the methods of the first and second aspects, the peptide sequence of the immune modulating peptide can consist of a sequence that has at least about 80% identity with SEQ ID NO:3. For example, the peptide sequence of the immune modulating peptide can consist of a sequence that has at least about 90% identity with SEQ ID NO:3. In another example, the peptide sequence of the immune modulating peptide can consist of a sequence that has at least about 95% identity with SEQ ID NO:3. In another example, the peptide sequence of the immune modulating peptide can consist of a sequence that has 100% identity with SEQ ID NO:3. The immune modulating peptide can have a length that is at least 80% the length of SEQ ID NO:3. For example, the immune modulating peptide can have a length that is 100% the length of SEQ ID NO:3.

In the methods of the first and second aspects, the peptide sequence of the immune modulating peptide can comprise SEQ ID NO:32 and SEQ ID NO:33. For example, the peptide sequence of the immune modulating peptide can consist of a sequence listed in Table 3. The immune modulating peptide can be cell permeable.

In the methods of the first and second aspects, the MANF family protein can be conserved dopamine neurotrophic factor (CDNF) or a fragment thereof.

In the methods of the first and second aspects, the peptide sequence of the immune modulating peptide can comprise a sequence that has at least about 80% identity with SEQ ID NO:6. For example, the peptide sequence of the immune modulating peptide can comprise a sequence that has at least about 90% identity with SEQ ID NO:6. In another example, the peptide sequence of the immune modulating peptide can comprise a sequence that has at least about 95% identity with SEQ ID NO:6. The immune modulating peptide can have a length that is at least 80% the length of SEQ ID NO:6. The immune modulating peptide can have a length that is 100% the length of SEQ ID NO:6.

In the methods of the first and second aspects, the peptide sequence of the immune modulating peptide can consist of a sequence that has at least about 80% identity with SEQ ID NO:6. For example, the peptide sequence of the immune modulating peptide can consist of a sequence that has at least about 90% identity with SEQ ID NO:6. In another example, the peptide sequence of the immune modulating peptide can consist of a sequence that has at least about 95% identity with SEQ ID NO:6. In another example, the peptide sequence of the immune modulating peptide can consist of a sequence that has 100% identity with SEQ ID NO:6. The immune modulating peptide can have a length that is at least 80% the length of SEQ ID NO:6. The immune modulating peptide can have a length that is 100% the length of SEQ ID NO:6.

In the methods of the first and second aspects, the peptide sequence of the immune modulating peptide can comprise SEQ ID NO:34 and SEQ ID NO:35. For example, the peptide sequence of the immune modulating peptide can consist of a sequence listed in Table 4. The neuroprotective peptide can be cell permeable.

In any of the methods of the first and second aspects, the replacement cells can comprise adult stem cells, amniotic stem cells, cord blood stem cells, induced pluripotent stem cells, or a combination thereof. The replacement cells can have been produced from adult stem cells, amniotic stem cells, cord blood stem cells, induced pluripotent stem cells, or a combination thereof. The replacement cells can comprise induced pluripotent stem cells. The replacement cells can have been produced from induced pluripotent stem cells. The replacement cells comprise autologous cells. The replacement cells comprise allogenic cells.

In any of the methods of the first and second aspects, the replacement cells can have been produced using a method that does not destroy an embryo capable of developing into a viable organism.

In any of the methods of the first and second aspects, treatment of neurodegenerative diseases can be excluded from the scope of the claims.

In any of the methods of the first and second aspects, the cell therapy method can be for treatment of a brain or spinal cord injury, a heart disease, a liver disease, baldness, missing teeth, a hearing impairment, a retinal disease, a muscular dystrophy, diabetes, an unhealed wound, or a combination thereof.

In some methods of the first and second aspects, the subject can have a retinal disorder. The retinal disorder can be macular degeneration, diabetic eye disease, age-related macular degeneration, branch retinal vein occlusion, central retinal vein occlusion, central retinal artery occlusion, central serous retinopathy, diabetic retinopathy, Fuchs' dystrophy, giant cell arteritis, glaucoma, hypertensive retinopathy, thyroid eye disease, iridocorneal endothelial syndrome, ischemic optic neuropathy, juvenile macular degeneration, macular edema, macular telangioctasia, marfan syndrome, optic neuritis, photokeratitis, retinitis pigmentosa, retinopathy of prematurity, stargardt disease, usher syndrome, Wolfram syndrome, or Leber Congenital Amaurosis. The replacement cells in these methods can comprise retinal pigment epithelium cells. The replacement cells can comprise retinal photoreceptor cells. The replacement cells in these methods can be administered by intravitreal administration, intracameral administration, conjunctival administration, intracorneal administration, intraocular administration, ophthalmic administration, retrobulbar administration, subconjunctival administration, or by transplant. The genetically modified cells in these methods can be administered by intravitreal administration, intracameral administration, conjunctival administration, intracorneal administration, intraocular administration, ophthalmic administration, retrobulbar administration, subconjunctival administration, or by transplant. The immune modulating peptide in these methods can be administered by intravitreal administration, intracameral administration, conjunctival administration, intracorneal administration, intraocular administration, ophthalmic administration, retrobulbar administration, or subconjunctival administration.

In some methods of the first and second aspects, the subject can have a hearing impairment. The hearing impairment can be caused by an ototoxic chemical, radiation, noise, age, or a combination thereof. The replacement cells in these methods can comprise hair cells. The replacement cells, the genetically engineered cells, or the immune modulating peptides in these methods can be administered by intratympanic administration, intracochlear administration, transtympanic injection, or a combination thereof.

In some methods of the first and second aspects, the subject can have a neurodegenerative disease. The neurodegenerative disease can be Parkinson's disease, amyotrophic lateral sclerosis, Alzheimer's disease, Lewy body disease, Huntington's disease, epilepsy, a memory disorder, an adult demyelinating disorder, a childhood myelin disease or glial disorder, or a combination thereof. The replacement cells in these methods can comprise astrocyte-oligodendrocyte glial progenitor cells, neuronal progenitor cells, dopaminergic neurons, medium spiny neurons, GABAergic neurons, cholinergic neurons, or a combination thereof. The replacement cells, the genetically engineered cells, or the immune modulating peptides in these methods can be administered by injection or infusion into a brain region.

In some methods of the first and second aspects, the subject can have a brain or spinal cord injury. The replacement cells in these methods can comprise glial progenitor cells, neural progenitor cells, bone marrow stem cells, or a combination thereof. The subject can have the brain injury and the replacement cells, the genetically engineered cells, or the immune modulating peptides can be administered by injection or infusion into a damaged brain region. The subject can have the spinal cord injury and the replacement cells, the genetically engineered cells, or the immune modulating peptides can be administered by injection or infusion into the spinal cord or the spinal canal.

In some methods of the first and second aspects, the subject can have a heart disease. The replacement cells in these methods can comprise skeletal myoblasts, bone marrow-derived cells, cardiac stem cells, mesenchymal stem cells, contractile cardiomyocytes, cardiac progenitors, endothelial cells, smooth muscle cells, or a combination thereof. The replacement cells, the genetically engineered cells, or the immune modulating peptides in these methods can be administered by intravascular infusion, intramyocardial injection, or by scaffold or patch-based epicardial delivery to the myocardium.

In some methods of the first and second aspects, the subject can have a liver disease. The replacement cells in these methods can comprise hepatocytes. The replacement cells, the genetically engineered cells, or the immune modulating peptides in these methods can be administered by infusion or injection through the portal vein into the liver, by direct injection into an extrahepatic site, or by transplantation of the replacement cells into a de-cellularized liver scaffold.

In some methods of the first and second aspects, the subject can have a hearing impairment. The hearing impairment can be caused by an ototoxic chemical, radiation, noise, age, or a combination thereof. The replacement cells in these methods can comprise hair cells. The replacement cells, the genetically engineered cells, or the immune modulating peptides in these methods can be administered by intratympanic administration, intracochlear administration, transtympanic injection, or a combination thereof.

In some methods of the first and second aspects, the subject can have a muscular dystrophy. The replacement cells in these methods can comprise satellite cells, myoblasts, bone marrow cells, blood vessel cells, skeletal myogenic progenitors, or a combination thereof. The replacement cells, the genetically engineered cells, or the immune modulating peptides in these methods can be administered by intramuscular injection or systemic injection.

In some methods of the first and second aspects, the subject can have diabetes. The replacement cells in these methods can comprise pancreatic islet cells. The replacement cells in these methods can comprise pancreatic beta cells. The replacement cells, the genetically engineered cells, or the immune modulating peptides in these methods can be administered by transcutaneous catheter infusion through the liver into the portal vein or by implantation in the pancreas.

In some methods of the first and second aspects, the subject can have an acute wound or slow healing or chronic wound. The replacement cells, the genetically engineered cells, or the immune modulating peptides in these methods can be administered directly to a wound site.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In the event that a term incorporated by reference conflicts with a term defined herein, this specification shall control.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: MANF is a hemocyte-derived damage response factor and promotes retinal repair in Drosophila. (A) Cartoon describing the experimental design and the current model for hemocyte mediated retinal repair in Drosophila. (B) Left, representative image of hemocyte smears from 3^{rd} instar larvae detecting MANF in Hml+ cells, identified by GFP expression. Nuclear staining is shown with DAPI. Right, western blot analysis of MANF and GFP (Hml-driven) proteins in cellular and plasma fractions from hemolymph of 3^{rd} instar larvae. (C) MANF relative mRNA levels detected by RT-qPCR in hemocyte samples collected from 3rd instar larvae of the designated genotypes and treatments (n≧5 for all conditions). (D) Western Blot analysis confirming MANF protein in cellular and plasma fractions from hemolymph of 3rd instar larvae following overexpression of Pvf-1 in the retina. Bottom graph represents the quantification of average relative levels from whole hemolymph samples (FIG. S2B, n=4). (E and G) Representative images of adult eye phenotypes from flies with the designated genotypes, after exposure of the right eye of P24 pupae to 17.5mJ of UV light. (F and H) Quantification of tissue loss by the average relative size of the UV-treated eye when compared to the untreated eye of the same fly (6<n<17 for each genotype). For all quantifications error bars represent s.e.m. and p-values are from student's t-test.

FIG. 2: PDGF-A/MANF damage-associated paracrine signaling is conserved in mammals. (A) Cartoon describing the cellular layer composition of the mouse eye. RPE, retinal pigmented epithelium; ONL, outer nuclear layer; OPL, outer plexiform layer; INL, inner nuclear layer; IPL. inner plexiform layer; GCL, ganglion cell layer. (B) Representative retinal images from C57BL/6J mice showing expression of PDGF-A by IHC, 6 h after exposure to 8 Klux of bright light for 1.5 h. Undamaged controls are shown for comparison. Nuclear staining is shown with DAPI. (C) MANF mRNA levels detected by RT-qPCR in retinal samples collected from C57BL/6J mice 12 h after exposure to 8 Klux of bright light for 1.5 h relative to undamaged controls (n=3 for both conditions). (D) Representative retinal images from C57BL/6J mice showing expression of CD11b and MANF by IHC, 12 h and 36 h after exposure to 8 Klux of bright light for 1.5 h. Undamaged controls are shown for comparison. Nuclear staining is shown with DAPI. Insets highlight CD11b+ cells detected in the plexiform layers in undamaged controls, in the vitreous 12 h after light exposure and within the ONL 36 h after light exposure and the presence or absence of MANF co-expression. Cartoons represent the regions of the eye shown in each section. (E, G and H) Representative retinal images from C57BL/6J mice showing expression of CD11b and MANF by IHC, one day after intravitreal injection of mrPDGF-AA or vehicle (PBS). Nuclear staining is shown with DAPI. Insets highlight CD11b+ cells detected in the plexiform layers (E), vitreous (E and G) and choroid blood vessels (H) and the presence or absence of MANF co-expression. Cartoon in E represents the experimental design. Cartoons in G and H represent the regions of the eye shown in each section. (F) Quantification of the average number of CD11b+ cells in the vitreous of C57BL/6J mice, per eye cryosection, one day after intravitreal injection of mrPDGF-AA protein (n=5, 3 sections per eye) or vehicle (PBS, n=6, 3 sections per eye). For all quantifications error bars represent s.e.m. and p-values are from student's t-test.

FIG. 3: MANF has neuroprotective and immune modulatory functions in the mammalian retina. (A) Representative retinal images from BALB/cJ mice, stained with TUNEL, two days after intravitreal injection of hrMANF or vehicle (PBS) and exposure to 5 klux of bright light for 1 h. Nuclear staining is shown with DAPI. Quantification of the average number of TUNEL+ nuclei per retinal field shown (hrMANF, n=8, 3 sections per eye; PBS, n=8, 3 sections per eye). (B) Representative retinal images from P16 Crx^{tvrm65} mice, stained with TUNEL, two days after intravitreal injection of hrMANF or vehicle (PBS). Nuclear staining is shown with DAPI. Quantification of the average number of TUNEL nuclei per eye cryosection is shown (hrMANF, n=6, 5 sections per eye; PBS, n=5, 5 sections per eye). (C) Representative retinal images from P21 Crx^{tvrm65} mice, stained with DAPI, seven days after intravitreal injection of hrMANF or vehicle (PBS). Dashed lines indicate the thickness of the ONL after MANF delivery for comparison. Quantification of the average number of nuclei rows in the ONL is shown (hrMANF, n=6, 5 sections per eye; PBS, n=6, 5 sections per eye). (D) Representative images retinal images from P28 Crx^{tvrm165} mice, stained with DAPI, fourteen days after intravitreal injection of MANF-secreting human fibroblasts (hfib-MANF) or control human fibroblasts (hfib-Cntrl). Dashed lines indicate the thickness of the ONL after hfib-MANF delivery for comparison. Quantification of the average number of nuclei rows in the ONL is shown (hfib-MANF, n=6, 5 sections per eye; hfib-Cntrl, n=6, 5 sections per eye). (E) Representative retinal images from P28 Crx^{tvrm65} mice, showing expression of CD11b and MANF (top panel) or Ym1 (lower panel) by IHC, fourteen days after intravitreal injection of hfib-MANF or hfib-Cntrl. Nuclear staining is shown with DAPI. Insets highlight CD11b+ cells and the presence or absence of MANF co-expression and Ym1 co-expression. Quantification of the average number of Ym1+ cells, per eye cryosection is shown (hfib-MANF, n=6, 5 sections per eye; hfib-Cntrl, n=6, 5 sections per eye). (F) Quantification of the average number of Arg1+ cells, per eye cryosection, in P21 Crx^{tvrm65} mice, seven days after intravitreal injection of hrMANF protein (n=6, 5 sections per eye) compared to vehicle (PBS, n=6, 5 sections per eye). (G) Left graph, quantification of the average number of Arg1+ cells, per eye cryosection, in BALB/cJ mice, two days after intravitreal injection of hrMANF or vehicle (PBS) and exposure to 5 klux of bright light for 1 h (hrMANF, n=8, 3 sections per eye; PBS, n=8, 3 sections per eye). Right graph, quantification of the percentage of MANF+ innate immune cells (CD11b+) in the retina of BALB/cJ mice after the same treatment (hrMANF, n=13 sections; PBS, n=5 sections). Corresponding representative images are shown in FIG. S5A-B. (H) Quantification of the average number of Ym1+ cells, per eye cryosection, in C57BL/6J mice, one day after intravitreal injection of mrPDGF-AA protein or vehicle (mrPDGF-AA, n=5, 3 sections per eye; PBS, n=6, 3 sections per eye). Corresponding representative images are shown in FIG. S5C. Cartoons represent the experimental design for each panel. For all quantifications error bars represent s.e.m. and p-values are from student's t-test.

FIG. 4: MANF regulates hemocyte activation in Drosophila. (A) Cartoon summarizing the phenotypes induced by MANF on hemocytes. (B) Representative IHC images of hemocyte smears from 3rd instar larvae of the designated genotypes and treatments, detecting Atilla protein. Hml+ cells are identified by GFP expression. Nuclear staining is shown with DAPI. For UV treatments, larvae were exposed to 50mJ of UV at 2nd instar stage and hemocytes collected 24 hours later. All analysis were performed after 24 h culture in control media (+, UV and UAS:MANF) or media supplemented with hrMANF protein. (C) Quantification of the percentage of Atilla+ cells (left) and High-GFP expressing cells (right) in the hemocyte population collected from 3rd instar larvae of the designated genotypes and treatments as described in (B) (n=5 for each genotype/treatment). (D and E) Hml (D) and Arg (E) relative mRNA levels detected by RT-qPCR in hemocyte samples collected from 3rd instar larvae of the designated genotypes (n≧3 for all conditions). (F) Representative IHC images of hemocyte smears from 3rd instar larvae of the designated genotypes, detecting MANF and Arg expression. Hml+ cells are identified by GFP expression. Graphs to the right are quantification of the percentage of MANF and Arg-expressing cells of the designated genotypes (approx. 120 cells/genotype, in 9 independent fields). For all quantifications error bars represent s.e.m. and p-values are from student's t-test.

FIG. 5: MANF-dependent hemocyte activation is required for neuroprotection. (A) KdelR and MANF relative mRNA levels detected by RT-qPCR in hemocyte samples collected from 3rd instar larvae of the designated genotypes (n=3 for all conditions). (B) Western Blot analysis shows presence of MANF protein in the plasma fraction of hemolymph collected from 3rd instar larvae expressing dsRNAi targeting KdelR transcripts in the hemocytes. (C) Arg relative mRNA levels detected by RT-qPCR in hemocyte samples collected from 3rd instar larvae of the designated genotypes (n=3 for all conditions). (D) Representative IHC images of hemocyte smears from 3rd instar larvae of the designated genotypes, detecting Atilla protein. Hml+ cells are identified by GFP expression. Nuclear staining is shown with DAPI. (E and F) Quantification of the percentage of Atilla+ cells in the hemocyte population collected from 3rd instar larvae of the designated genotypes (E, n≧3 for each genotype) and cultured for 24 h in presence of hrMANF protein or control media (F, n=3 for each genotype/treatment). (G) Cartoon summarizing the effects of KdelR KD on MANF-induced hemocyte phenotypes. (H) Representative images of adult eye phenotypes from flies with the designated genotypes, after exposure of the right eye of P24 pupae to 17.5mJ of UV light. (I) Quantification of tissue loss by the average relative size of the UV-treated eye, when compared to the untreated eye of the same fly (5<n<20 for each genotype). For all quantifications error bars represent s.e.m. and p-values are from student's t-test. (35) and (36) correspond to two independent dsRNAi expressing lines targeting KdelR transcripts.

FIG. 6: MANF enhances efficiency of integration of transplanted photoreceptors. (A) Cartoon representing the trans-corneal subretinal injection method. (B) Representative images of an integration site of Nrl-GFP donor photoreceptors one week after transplantation, analyzed by IHC for CD11b, MANF and GFP expression. (C) Quantification of the number of MANF+CD11b+ cells/field in integration sites vs. sites of no integration (10 fields per condition, all fields contained cells in the subretinal space). (D) Representative images and quantification of integration in C57BL/6J mice, analyzed by IHC for GFP expression, seven days after subretinal injection of P21 Nrl-GFP donor photoreceptors (PhR) supplemented with hrMANF protein (n=8) or vehicle (PBS, n=9). (E) Representative images and quantification of integration in wild-type (wt) or Crx^{tvrm65} mice (C57BL/6J background for both), analyzed by IHC for GFP expression, seven days after subretinal injection of P7 Nrl-GFP donor photoreceptors (PhR) supplemented with hrMANF protein (hrMANF) (n=4) or vehicle (PBS, n=4). For all quantifications error bars represent s.e.m. and p-values are from student's t-test.

FIG. 7: Model for the evolutionarily conserved immune modulatory function of MANF and its implication in tissue repair and regeneration. (A) In Drosophila (left) or mouse (right) the damaged retina secretes Pvf-1/PDGF-A which acts on innate immune cells—hemocytes in Drosophila or microglia/macrophages in mice—to induce MANF expression. Innate immune cell-derived MANF acts in an autocrine loop to promote phenotypic changes—atilla and arginase expression in hemocytes or M2 phenotypes (Ym1 and arginase1 expression) in microglia/macrophages. The resulting phenotypic transformations support tissue repair and regeneration of the retina. (B) MANF supplementation is an enhancer of retinal regenerative therapies by increasing the integration efficiency of exogenously supplied photoreceptors for retinal repair.

FIG. 8: RNA sequencing of hemocyte samples after epithelial UV damage, related to FIG. 1. (A) Venn diagram showing the analysis of the four datasets of RNAseq data leading to the identification of MANF. Candidate genes were selected by the following additive criteria: 1) Induction by UV in Hml>GFP larvae greater than 2 (pink; top-left circle); 2) Induction by UV in Hml>PvRRNAi larvae smaller than 2 (yellow; top right circle) and 3) change in expression levels by PvR loss of function smaller than 2 (green; bottom circle). Table on the right shows examples of RPKM values for genes meeting all criteria, and MANF is highlighted in red (light grey), (B) Table showing examples of RPKM values for genes encoding ribosomal proteins in the four datasets analyzed. (C) Relative RPKM values for PvR in hemocytes from larvae of the designated genotypes.

FIG. 9: Hemocyte-derived MANF and retinal repair in Drosophila, related to FIG. 1. (A) PvR relative mRNA levels detected by RT-qPCR in hemocyte samples collected from 3rd instar larvae of the designated genotypes (n=3 for all conditions). (B) Western Blot analysis of MANF and Actin proteins in whole hemolymph collected from 3rd instar larvae overexpressing Pvf-1 in the retina. (C) MANF relative mRNA levels detected by RT-qPCR in hemocyte samples collected from 3rd instar larvae of the designated genotypes (n≧6 for all conditions). (D) Representative images of adult eye phenotypes from GMR:Grim flies. Code color indicates label to the severity of the phenotype and corresponds to the bar graph on the right. Quantifications on the right, performed independently for males and females, indicate the distribution of phenotypes for each of the genotypes indicated. UAS:MANF/Cyo and Hml:Ga14/UAS:MANF flies are siblings. MANF overexpression in the hemocytes rescued the severity in eye phenotypes from GMR:Grim flies. (E) Representative images of adult eye phenotypes from GMR:RhlG69D flies. MANF overexpression in hemocytes rescued the severity in eye phenotypes from GMR:Rh1G69D flies. Quantifications on the right, performed independently for males and females, indicate the average eye size, quantified as described for UV experiments but using lateral views (as shown in image). MANF overexpression in the hemocytes rescued eye size from GMR:RhlG69D female flies. (F) MANF relative mRNA levels detected by RT-qPCR in hemocyte samples collected from 3rd instar larvae of the designated genotypes (n≧3 for all conditions). (G) Representative images of adult eye phenotypes from flies of the designated genotypes, after exposure of the right eye of P24 pupae to 17.5mJ of UV light. Quantifications on the right are as in FIG. 1H. Quantifications of necrotic tissue were performed in lateral views using ImageJ to determine the percentage of eye surface covered by black tissue. For all quantifications error bars represent s.e.m. and p-values are from student's t-test.

FIG. 10: Light damage of the mammalian retina, related to FIG. 2. (A) Cartoon describing the experimental design for the light damage experiment. (B) Representative retinal images from C57BL/6J mice, stained with TUNEL, two days after exposure to 8 klux of bright light for 1.5 h. Undamaged controls are shown for comparison. Nuclear staining is shown with DAPI. (C) Representative retinal images from C57BL/6J mice, showing expression of Iba-1 and CD11b by IHC, 12 h, 24 h and 36 h after exposure to 8 Klux of bright light for 1.5 h. Undamaged controls are shown for comparison. Nuclear staining is shown with DAPI. (D) Representative retinal images from C57BL/6J mice, showing expression of GFAP and MANF by IHC, 12 h after exposure to 8 Klux of bright light for 1.5 h. MANF expression in undamaged controls is shown for comparison. Nuclear staining is shown with DAPI. (E) Quantification of the average number of TUNEL+ nuclei per field of retinal epithelium on cryosections from BALB/cJ mice, two days after exposure to 5 klux of bright light for 1 h. Quantification in undamaged retinas is shown for comparison. (n=4 per condition, 3 sections per eye). For all quantifications error bars represent s.e.m. and p-values are from student's t-test.

FIG. 11: MANF is neuroprotective in Rd1 retinas, related to FIG. 3. (A) Representative retinal images from Pde6b^{Rd1} mice, stained with TUNEL five days after intravitreal injection of hrMANF or vehicle (PBS). Nuclear staining is shown with DAPI. Bar graph to the right is the quantification of the average number of TUNEL+ nuclei per field of retinal epithelium in the same conditions (hrMANF, n=3, 3 sections per eye; PBS, n=3, 3 sections per eye). (B) Representative retinal images from Pde6b^{Rd1} mice stained with Recoverin (bottom row), five days after intravitreal injection of hrMANF or vehicle (PBS). Nuclear staining is shown with DAPI (top row) in the same conditions. Dashed lines indicate the thickness of the ONL after MANF delivery for comparison. Bar graph to the right is the quantification of the average number of nuclei rows in the ONL of retinal epithelium on cryosections from Pde6b^{Rd1} mice, five days after intravitreal injection of hrMANF (n=3, 3 sections per eye) or vehicle (PBS, n=3, 3 sections per eye). (C) Representative images of direct GFP fluorescence on a field of human fibroblasts infected with GFP-MANF expressing lentivirus. (D) Western Blot analysis of MANF and GFP proteins in the media and cell extracts collected from the cultures shown in (C). GFP-MANF fusion protein is detected in the cell extracts and in the supernatant media, indicating that it is secreted. Cartoons on A and B represent the experimental design. For all quantifications error bars represent s.e.m. and p-values are from student's t-test.

FIG. 12: MANF intravitreal delivery promotes MANF expression, related to FIG. 3. (A and B) Representative retinal images from BALB/cJ mice, showing expression of CD11b and MANF (A) or Arg1 (B) by IHC, two days after intravitreal injection of hrMANF or vehicle (PBS) and exposure to 5 klux of bright light for 1 h. Nuclear staining is shown with DAPI. Insets highlight CD11b+ cells and the presence or absence of MANF co-expression and Arg1 co-expression. (C) Representative retinal images from C57BL/6J mice showing expression of CD11b and Ym1 by IHC, one day after intravitreal injection of mrPDGF-AA or vehicle (PBS). Nuclear staining is shown with DAPI. Insets highlight CD11b+ cells and the presence or absence of Ym1 co-expression. Cartoons represent the experimental design for each panel.

FIG. 13: Profiling of innate immune cells phenotypes after MANF intravitreal delivery, related to FIG. 3. (A) Representative retinal images from Crx^{tvrm65} mice, showing expression of Gr-1, F4/80, CD68, MANF and Arg1 by IHC, fourteen days after intravitreal injection of MANF-secreting human fibroblasts (hfib-MANF). Gr-1 is a neutrophil marker (red label; top 3 panels)) and F4/80 and CD68 are pan-macrophage/monocyte markers (blue label; bottom 6 panels). Areas shown are located in the vitreous. Cartoon represents the experimental design. (B) Quantification of the percentage of cells in the vitreous that expressed each of the makers defined and shown in (A). For each population, the number of cells co-expressing MANF and Arg1 are also indicated (n=10 sections from 6 eyes). This summary shows that the majority of cells found in the vitreous are monocytes/macrophages, a small fraction are neutrophils and all this innate immune cell types express MANF.

FIG. 14: Stress induced MANF mediates changes in hemocyte populations. (A) Representative images of hemocytes under control conditions (left column), after UV damage (middle column), or after MANF overexpression (right column); nuclear staining is by DAPI (top row) and Hml is shown by GFP (bottom row). (B) Quantitation of Hml and GFP expression with or without MANF overexpression (left) and with or without UV damage (right). (C) Western blots for GFP (Hml) in cells and in the plasma with or without overexpression of MANF.

FIG. 15: MANF exogenous delivery changes the immune environment after light damage of the mouse retina. (A) Mouse retinal images under control conditions (left column), after light damage (middle column), and after light damage with prior exogenous delivery of MANF; MANF immunoreactivity (bottom row), CD11b immunoreactivity (middle row) and an overlay (top row) are shown. (B) & (C) Quantification of the percent of MANF positive CD11b cells under control, light damage, and light damage plus MANF injection. (D) Representative retinal images after light damage with prior MANF injection showing MANF expressing amoeboid shape microglia/macrophages invading the retina with detection of M2 (alternative activated/tissue protective macrophage) markers.

FIG. 16: MANF exogenous delivery changes the immune environment in a mouse model of retinal degeneration. (A) Mouse retinal images in Crx−/− mice without treatment (left column), after PBS injection (middle column), and MANF injection; MANF immunoreactivity (bottom row), CD11b immunoreactivity (middle row) and an overlay (top row) are shown. (B) Representative retinal images in Crx−/− mice after MANF injection showing MANF expressing amoeboid shape microglia/macrophages invading the retina with detection of M2 (alternative activated/tissue protective macrophage) markers.

FIG. 17: Bright filed images collected at 16 h are shown on the top panel, showing a partial recovery in morphology of LPS treated cells, when MANF is co-supplemented (compare LPS vs. LPS+ MANF). RNA samples were collected at the designated times and relative mRNA levels of two markers of M2 (alternative) macrophage polarization (Ym1 and CD206) were quantified. In the absence (top graph) and presence (bottom graph) of a pro-inflammatory agent, MANF treatment induced the expression of markers of M2 activation in Raw264.7 Macrophages. N=3 for all time points and treatments.

### DETAILED DESCRIPTION OF THE INVENTION

Cell therapy is any therapy in which cellular material is injected into a subject. Cell therapy can be targeted at many clinical indications in multiple organs and by several modes of cell delivery. There are two main principles by which injection of cells can facilitate therapeutic action: cell replacement and immunomodulation to stimulate self-healing. These principles are not necessarily mutually exclusive. In cell replacement therapy, live cells are locally or systemically injected into a subject, after which they integrate into the site of injury and replace damaged or injured tissue. In many cases, when cell therapy is used for immunomodulation, cells that release soluble factors that facilitate self-healing of the organ or region are injected.

Cell therapy, either for cell replacement or immunomodulation, has met with numerous challenges. For example, aged and damaged tissues often provide a poor environment for integration (for example, due to inflammation in that microenvironment), reducing the efficiency of cell replacement therapy. In addition, cell viability represents a challenge for both replacement cells and cells secreting pro-regeneration factors in immunomodulation cell therapy.

The teachings and experiments presented herein have identified mesencephalic astrocyte-derived neurotrophic factor (MANF) as a co-adjuvant that significantly improves cell therapy by modulating the immune-microenvironment to improve cell viability and integration efficiency. In addition, MANF can be used by itself for immunomodulation, such as preventing and/or decreasing inflammation in areas that injured (e.g., environmentally caused injury or degeneration). Disclosed herein are methods and compositions for the use of MANF family proteins, and fragments thereof, in cell therapy.

General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Antibodies: A Laboratory Manual, Second edition, Cold Spring Harbor Laboratory Press, Cold Spring Harbor, N.Y. (Greenfield, ed., 2014), Beaucage et al. eds., Current Protocols in Nucleic Acid Chemistry, John Wiley & Sons, Inc., New York, 2000, (including supplements through 2014) and Gene Transfer and Expression in Mammalian Cells (Makrides. ed., Elsevier Sciences B.V., Amsterdam, 2003).

### Definitions

Unless defined otherwise herein, all technical and scientific terms used herein have the same meaning as commonly understood by one of ordinary skill in the art to which this invention pertains. For the purposes of clarity and a concise description, features can be described herein as part of the same or separate embodiments; however it will be appreciated that the scope of the invention may include embodiments having combinations of all or some of the features described.

The terminology used herein is for the purpose of describing particular embodiments only and is not intended to be limiting of the invention.

“Immunomodulation” or “immunomodulatory” can refer to an alteration in the overall immunoreactivity of the immune system in a mammal, or alteration in the response of a cell, relative to an untreated control of the same type, upon treatment with an agent, such as a polypeptide or nucleic acid molecule of the present invention, or fragments and analogs thereof. Immunomodulation can be assayed using immune cells, for example, B cells, T cells, antigen-presenting cells, or any other cell that is involved in immune function Immunomodulation can also be assayed by determining expression and/or activity of immune-related genes and proteins, or immune-related compounds, such as cytokines, cytokine receptors, immunoglobulins, etc.

“Immunosuppression” refers to a decrease in the overall immunoreactivity of the immune system upon administration of an immunomodulator in comparison to the immunoreactivity of an immune system that has not been contacted with the particular immunomodulator “Immunostimulation” refers to a increase in the overall immunoreactivity of the immune system upon administration of an immunomodulator in comparison to the immunoreactivity of an immune system that has not been contacted with the particular immunomodulator. “Decreasing T cell stimulation” means lowering the level of T cell stimulation as measured by, for example, a chromium release assay. “Decreasing inflammation” means decreasing the number of inflammatory cells (leukocytes, for example eosinophils) in the target tissue by, preferably, two-fold. By “cell proliferation” is meant the growth or reproduction of similar cells. By “apoptosis” is meant the process of cell death where a dying cell displays a set of well-characterized biochemical hallmarks which include cytolemmal blebbing, cell soma shrinkage, chromatin condensation, and DNA laddering.

“Immunomodulator” refers to an agent that induces an immunomodulatory effect or alteration (i.e., immunosuppression, immunostimulation, etc.) as measured, for example, by an alteration of virulence in mutated viruses or a variety of immunoassays well known in the art (for example, chemotaxis assays as described herein). For example, in the present invention, an immunomodulator may elicit an altered level of immune function. An “anti-inflammatory” agent is an immunomodulatory agent capable of decreasing the overall inflammation or immune function upon administration to an individual. In some embodiments MANF is an immunomodulator. In some embodiments MANF is an anti-inflammatory agent.

All genes and gene products (including RNA and proteins), and their respective names, disclosed herein are intended to correspond to homologs from any species for which the compositions and methods disclosed herein are applicable. When a gene or gene product from a particular species is disclosed, it is understood that this disclosure is intended to be exemplary only and is not to be interpreted as a limitation unless the context in which it appears clearly indicates otherwise. For example, the genes and gene products disclosed herein, which in some embodiments relate to mammalian (including human) nucleic acid and/or amino acid sequences, are intended to encompass homologous and/or orthologous and/or paralogous genes and gene products from other animals including, but not limited to, other mammals, fish, reptiles, amphibians, birds, and other vertebrates.

As used herein, the terms “polypeptide,” and “peptide,” are equivalent and mutually interchangeable. They refer to any amino acid chain of two or more amino acids, including native peptides, degradation products, synthetically synthesized peptides, or recombinant peptides; and include any post-translational modifications thereto (for example phosphorylation or glycosylation). Polypeptides include modified peptides, which may have, for example, modifications rendering the peptides more stable or less immunogenic. Such modifications can include, but are not limited to, cyclization, N-terminus modification, C− terminus modification, peptide bond modification, backbone modification and residue modification. Acetylation—amidation of the termini of the peptide (e.g., N-terminal acetylation and C− terminal amidation) can increase the stability and cell permeability of the peptides.

“Post-translational modification” can mean any change to a polypeptide or polypeptide fragment during or after synthesis. Post-translational modifications can be produced naturally (such as during synthesis within a cell) or generated artificially (such as by recombinant or chemical means). A “protein” can be made up of one or more polypeptides.

As used herein, the term “fragment” refers to a portion of a compound. For example, when referring to a protein, a fragment is a plurality of consecutive amino acids comprising less than the entire length of the polypeptide.

The disclosure of a particular sequence should be understood as disclosure of all fragments of a sequence. A fragment of a sequence can be defined according to a percent length of a reference sequence (e.g., a reference protein or peptide sequence). For example, a fragment of a sequence (e.g., protein or peptide sequence) can have a length that is at least about 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the length of the reference sequence. In another example, a fragment of a sequence (e.g., protein or peptide sequence) can have a length that is at most about 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the length of the reference sequence. In another example, a fragment of a sequence (e.g., protein or peptide sequence) can have a length that is about 1-99%, 2-99%, 5-99%, 10-99%, 20-99%, 30-99%, 40-99%, 50-99%, 60-99%, 70-99%, 80-99%, 90-99%, 2-90%, 5-90%, 10-90%, 20-90%, 30-90%, 40-90%, 50-90%, 60-90%, 70-90%, 80-90%, 5-80%, 10-80%, 20-80%, 30-80%, 40-80%, 50-80%, 60-80%, 70-80%, 10-70%, 20-70%, 30-70%, 40-70%, 50-70%, 60-70%, 20-60%, 30-60%, 40-60%, 50-60%, 30-50%, 40-50%, or 30-40% of the length of the reference sequence. Fragments can also be defined as have a percent identity to a reference sequence; for example a fragment can have length that is less than the reference sequence and a percent identity of the reference sequence.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SUM J. Applied Math. 48: 1073.

The disclosure of any particular sequence herein should be interpreted as the disclosure of all sequences sharing a percent identity with the sequence. A sequence can be defined herein according to a percent identity with a reference sequence. For example, the sequence can have at least about: 50%, 55%, 60%, 65%, 70%, 75%, 80%, 85%, 90%, 95%, 96%, 97%, 98%, 99%, or 100% identity with the reference sequence. In another example, the sequence can have about: 50-60%, 50-75%, 50-80%, 50-85%, 50-90%, 50-95%, 50-97%, 50-99%, 50-100%, 60-75%, 60-80%, 60-85%, 60-90%, 60-95%, 60-97%, 60-99%, 60-100%, 75-80%, 75-85%, 75-90%, 75-95%, 75-97%, 75-99%, 75-100%, 80-85%, 80-90%, 80-95%, 80-97%, 80-99%, 80-100%, 85-90%, 85-95%, 85-97%, 85-99%, 85-100%, 90-95%, 90-97%, 90-99%, 90-100%, 95-97%, 95-99%, 95-100%, 97-99%, 97-100%, or 99-100% identity with the reference sequence. Such sequences can be called variants of the reference sequence.

A “variant” of a polypeptide comprises an amino acid sequence wherein one or more amino acid residues are inserted into, deleted from and/or substituted into the amino acid sequence relative to another polypeptide sequence. The substituted amino acid(s) can be conservative substitutions or non-conservative substitutions, depending upon the context. Variants include fusion proteins.

Conservative substitutions are substitutions of one amino acid with a chemically similar amino acid. The following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

In making changes to the peptides and proteins disclosed herein, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein or peptide can be considered in designing variants of a protein or peptide. Certain amino acids can be substituted for other amino acids having a similar hydropathic index or score and still retain a similar biological activity. In making changes based upon the hydropathic index, the substitution of amino acids whose hydropathic indices are within ±2, ±1, or ±0.5 are included.

The substitution of like amino acids can also be made effectively on the basis of hydrophilicity. In certain embodiments, the greatest local average hydrophilicity of a protein or peptide, as governed by the hydrophilicity of its adjacent amino acids, correlates with a biological property of the protein or peptide.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, the substitution of amino acids whose hydrophilicity values are within ±2, ±1, ±0.5 are included.

As used herein, the term “subject” refers to any animal (e.g., mammals, birds, reptiles, amphibians, fish), including, but not limited to, humans, non-human primates, rodents, and the like, which is to be the recipient of a particular treatment. Typically, the terms “subject” and “individual” may be used interchangeably herein in reference to a subject.

As used herein, the term “administering” refers to providing an amount of a chemical or biological compound or pharmaceutical composition to a subject. The chemical or biological compound can be administered alone, but may be administered with other compounds, excipients, fillers, binders, carriers or other vehicles selected based upon the chosen route of administration and standard pharmaceutical practice. Administration may be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; ear drops; sprays, including nasal sprays; other proteins and peptides; synthetic polymers; microspheres; nanoparticles; and the like.

The active ingredients (e.g., chemicals or biological compounds or pharmaceutical compositions) disclosed herein can also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. Moreover, the packaging material may be biologically inert or lack bioactivity, such as plastic polymers, silicone, etc. and may be processed internally by the subject without affecting the effectiveness of the active ingredient.

“Cell therapy” as used herein is any therapy where cellular material (e.g., live cells, tissues) are injected into a subject. Cell therapy can include treatments where cells are injected into a subject to replace damaged or diseased tissue. Cell therapy can include treatments where cells that are capable of releasing soluble factors to improve a healing or regenerative response are injected into a subject.

An “adjuvant” is a pharmacological or immunological active ingredient that modifies the effects of another active ingredient. For example, a cell therapy adjuvant can be a pharmacological or immunological active ingredient that increases the effectiveness of the cell therapy. The increased effectiveness can be, e.g., due to an increased viability or an increased integration efficiency of the injected cells.

The term “effective amount,” as applied to the active ingredient(s) (e.g., compound(s), biologics and pharmaceutical compositions) described herein, means the quantity necessary to render the desired therapeutic result. For example, an effective amount is a level effective to treat, cure, or alleviate the symptoms of a disorder for which the therapeutic compound, biologic or composition is being administered. In another example, an effective amount of an adjuvant for cell therapy is an amount effective to increase the viability or integration efficiency of the injected cells or tissues. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

As used herein, “disorder” refers to a disorder, disease or condition, or other departure from healthy or normal biological activity, and the terms can be used interchangeably. The terms would refer to any condition that impairs normal function. The condition may be caused by sporadic or heritable genetic abnormalities. The condition may also be caused by non-genetic abnormalities. The condition may also be caused by injuries to a subject from environmental factors, such as, but not limited to, cutting, crushing, burning, piercing, stretching, shearing, injecting, or otherwise modifying a subject's cell(s), tissue(s), organ(s), system(s), or the like.

As used herein, “treatment” or “treating” refers to arresting or inhibiting, or attempting to arrest or inhibit, the development or progression of a disorder and/or causing, or attempting to cause, the reduction, suppression, regression, or remission of a disorder and/or a symptom thereof. Various clinical and scientific methodologies and assays can be used to assess the development or progression of a disorder, and similarly, various clinical and scientific methodologies and assays can be used to assess the reduction, regression, or remission of a disorder or its symptoms. Additionally, treatment can be applied to a subject or to a cell culture.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

As used herein, the term “about” means plus or minus 10% of the indicated value. For example, about 100 means from 90 to 110.

Introduction

Regenerative therapies using stem cell-derived cells hold promise for the treatment of a range of diseases and disorders, including diabetes, liver diseases, neurologic disorders, spinal cord injury, heart disease, and retinal diseases. Induced pluripotent stem cells (PSC) directed to various cell fates can serve as a virtually unlimited supply of materials for treating these diseases and disorders. These cell therapy techniques, however, face numerous challenges. For example, aged and diseased tissues typically provide a poor microenvironment for integration of new cells. Further, cell loss after transplantation reduces the efficiency of cell therapy.

Tissue repair involves sophisticated interactions between damaged tissues and innate immune cells Immune cells can promote wound healing, clear damaged cells and fight infection, yet their excessive/aberrant activation can cause inflammatory conditions that limit repair and regeneration. As shown in the Examples presented herein, tissue damage induces the expression of Mesencephalic Astrocyte-derived Neurotrophic Factor (MANF) in innate immune cells. MANF, in turn, enhances M2 polarization of immune cells, resulting in an anti-inflammatory microenvironment that improves neuroprotection and tissue repair.

The experiments presented herein highlight the benefit of immunomodulation using MANF for improving tissue repair and regeneration, and identify a new use for MANF family proteins as adjuvants for enhancing regenerative therapies (e.g., cell therapies). The MANF family proteins include mesencephalic astrocyte-derived neurotrophic factor (MANF) and cerebral dopamine neurotrophic factor (CDNF). Both MANF and CDNF can enhance the survival of dopaminergic neurons in vitro and in vivo. As shown in the experiments presented herein, MANF also significantly reduces tissue loss in both fly and mouse models of retinal degeneration. Further, the loss of MANF in flies significantly increased the tissue degeneration response in the retina upon exposure to ultraviolet light. Not only was MANF protective of retinal tissue, but it also promoted cell integration in the mammalian retina.

The evolutionary conservation of MANF, as well as its broad damage-stimulated expression, supports a wide-ranging role in tissue repair and regeneration processes for MANF. Accordingly, disclosed herein are methods and compositions for the use of MANF family proteins as an adjuvant to improve the efficiency of cell therapy.

Cell Therapy

Cell therapy involves the injection of cellular materials into a subject to achieve a therapeutic outcome. Generally, cell therapy involves the injection of living cells or tissues to serve as replacements for damaged or diseased tissue, or to provide a source of soluble factors such as cytokines, chemokines, or growth factors to facilitate self-healing. Cell therapy can be targeted at many clinical indications in multiple organs and by several modes of cell delivery.

Replacement Cells

Cell therapy can involve the administration (e.g., injection) of cells to serve as replacements for damaged or diseased tissue. As such, disclosed herein are methods and compositions for administering replacement cells to a subject in need thereof, the methods comprising the use of an immune modulating peptide as an adjuvant. Replacement cells can be stem cells. Replacement cells can be produced from stem cells. Replacement cells can be somatic cells isolated from a donor (e.g., a cadaver or organ donor).

Stem Cells

Cell therapy can involve the use of stem cells. Stem cells are undifferentiated cells that can differentiate into specialized cell types and can self-renew by dividing to produce more stem cells. The stem cells can be administered in an undifferentiated state or a partially differentiated state, or they can be differentiated into a target cell type prior to administration. The stem cells can also be used to produce replacement tissue.

Stem cells can be classified based upon their potency or potential to differentiate into different cell types. Totipotent stem cells can differentiate into any cell type, and are capable of producing an entire organism. Pluripotent stem cells can differentiate into nearly all cells, generally including cells from all three germ layers in vertebrates (e.g., endoderm layer, mesoderm layer, and ectoderm layer). Multipotent stem cells are stem cells capable of differentiating into a closely related family of cells. Oligopotent stem cells can differentiate into only a few cell types. And unipotent cells can only produce one cell type.

There are many different sources of stem cells, including embryonic stem cells (ES), fetal stem cells, adult stem cells, amniotic stem cells, cord blood stem cells, and induced pluripotent stem cells. Embryonic stem (ES) cells are pluripotent stem cells derived from the inner cell mass of a blastocyst, an early-stage embryo. Fetal stem cells are located in the organs of fetuses and can be either from the tissue of the fetus proper (fetal proper stem cells) or from extraembryonic membranes (extraembryonic fetal stem cells). Extraembryonic fetal stem cells are acquired after birth and are generally not distinguished from adult stem cells. Adult stem cells are stem cells that maintain and repair the tissue in which they are found and can vary in potency depending upon the source. Pluripotent adult stem cells can be found in the umbilical cord blood, bone marrow, and a few other tissues. Most adult stem cells are multipotent and generally referred to by their tissue of origin (e.g., mesenchymal stem cells, adipose-derived stem cells, endothelial stem cells, dental pulp stem cells, etc) Amniotic stem cells are found in the amniotic fluid and are multipotent. Cord blood-derived multipotent stem cells are isolated from mammalian cord blood. Induced pluripotent stem cells are adult cells that have been reprogrammed to be pluripotent.

The use of certain types of stem cells, either directly or to produce other cell types, can be specifically excluded from the scope of the claims, if indicated. For example, the scope of the claims can be limited to exclude the use of any type of stem cells that involves the destruction of an embryo capable of developing into a mature organism.

The methods and compositions for cell therapy disclosed herein can use induced pluripotent stem cells. Induced pluripotent stem cells (PSC) can be generated by reprogramming adult (somatic) cells to a pluripotent state by transient expression of pluripotency factors. These cells can self-renew indefinitely and are able to differentiate into any cell lineage.

The induced pluripotent stem cells employed in the methods and compositions disclosed herein can be autologous PSCs. The ability to generate PSC from individual patients and differentiate them into an unlimited supply of tissue and organ-specific cells has an advantage in that these cells would be capable of circumventing immunologic rejection following transplantation. Alternatively, the PSCs employed in the methods and compositions disclosed herein can be allogenic, wherein the cell donor is different from the recipient.

Induced pluripotent stem cells can be produced by reprogramming somatic cells. The somatic cells used to produce PSCs can generally include any live somatic cells. Exemplary somatic cells include, but are not limited to, fibroblasts; epithelial cells; muscle cells (e.g., skeletal muscle cells, visceral muscle cells, cardiac muscle cells); hepatic cells; bone cells; vascular endothelial cells; brain neurons; glia (e.g., oligodendroglia and astroglia); primary, secondary, tertiary, and subsequent spheres derived from various cells including brain neurons, glia, and cancer cells; peripheral blood- or bone marrow-derived mononuclear cells; granular leukocytes, and lymphocytes; keratinocytes, osteoblasts; osteoclasts; gastric epithelial cells; liver epithelial cells; small and large intestinal tract epithelial cells; pancreatic cells (endocrine cells such as alpha cells and beta cells, and exocrine cells); adult stem cells (e.g., adipose-derived stem cells (ADSCs), hematopoietic stem cells, neural stem cells, mesenchymal stem cells).

Methods of producing PSCs can include reprogramming somatic cells to express one or more reprogramming genes. Such reprogramming genes can be selected from the group consisting of NANOG, SOX2, OCT3/4, KLF4, LIN28, c-MYC, and Glis1. The somatic cells can be reprogrammed by introducing the one or more reprogramming genes to the cell exogenously (e.g., by viral delivery). The somatic cells can be reprogrammed by using one or more factors to induce expression of the endogenous versions of the one or more reprogramming genes. For example, microRNAs can be used to induce the expression of the endogenous reprogramming genes. The somatic cells can be reprogrammed by mimicking the function of the one or more reprogramming genes. For example, small molecules or exogenous proteins can be introduced to the somatic cells that stimulate the same signaling cascades as the one or more reprogramming genes.

Successfully reprogrammed somatic cells (e.g., induced pluripotent stem cells) can be identified based upon, for example, cellular biological properties including morphology, growth properties, detection of cell surface markers (e.g., SSEA-3, SSEA-4, TRA-1-60, TRA-1-81, TRA-2-49/6E, or Nanog), expression of stem cell genes (e.g., Oct-3/4, Sox2, Nanog, GDF3, REX1, FGF4, ESG1, DPPA2, DPPA4, or hTERT), or high telomerase activity. Successfully reprogrammed somatic cells (e.g., induced pluripotent stem cells) can be identified based upon, for example, the ability to differentiate into different cell types (e.g., neural cell types, cardiac cell types, etc.), the formation of teratomas in immunodeficient mice, or the formation of embryoid bodies in culture.

The specific cell types used in the cell therapy methods and compositions will depend upon the indication to be treated.

Indications

The methods and compositions disclosed herein can be useful for the treatment of subjects having a wide range of indications. For example, the cellular therapy methods, using an immune modulating peptide as an adjuvant, can be useful in the treatment of neurodegenerative diseases, brain and spinal cord injury, heart disease, liver disease, baldness, replacement of missing teeth, hearing impairment, retinal diseases (e.g., blindness and vision impairment), muscular dystrophies, diabetes, wounds (e.g., slow healing or chronic wounds, acute wounds), and may other types of diseases and disorders.

Neurodegenerative Diseases

A subject treated with the cell therapy compositions and methods disclosed herein can have a neurodegenerative disease. For example, the subject can have Parkinson's disease, amyotrophic lateral sclerosis, Alzheimer's disease, Lewy body disease, Huntington's disease, epilepsy, memory disorders, adult demyelinating disorders (e.g., multiple sclerosis, white matter stroke, Krabbe disease, metachromatic leukodystrophy, Tay-Sachs disease), childhood myelin disease and glial disorders, or a combination thereof.

The replacement cells used in cell therapy to treat neurodegenerative diseases can depend upon the disease being treated. The replacement cells can be stein cells (e.g., induced pluripotent stem cells), cells produced from stem cells, or cells obtained from a donor. The replacement cells can be astrocyte-oligodendrocyte glial progenitor cells (e.g., for treating adult demyelinating disorders or childhood myelin disease and glial disorders). The replacement cells can be neuronal progenitor cells (e.g., to treat Parkinson's disease, Huntington's disease, epilepsy, memory disorders). The replacement cells can be differentiated neurons. For example, the replacement cells can be dopaminergic neurons (e.g., to treat Parkinson's disease), medium spiny neurons (e.g., to treat Huntington's disease), GABAergic neurons (e.g., to treat epilepsy), cholinergic neurons (e.g., to treat memory disorders), or a combination thereof. Alternatively, the scope of the claims can be limited to exclude treatment of a neurodegenerative disease, if indicated. For example, the scope of the claims can be limited to exclude Parkinson's disease, if indicated.

Cells and immune modulating adjuvants can be administered in subjects with neurodegenerative diseases by, for example, by injection or infusion into the brain at or near a sight of injury or cell loss. Injection or infusion can be performed using a catheter. Injection or infusion can be accomplished with convection enhanced delivery.

Brain and Spinal Cord Injury

A subject treated with the cell therapy compositions and methods disclosed herein can have a brain or spinal cord injury. For example, the subject can have paralysis due to damage to the spinal cord, or brain damage due to stroke or heart attack. Replacement cells used in cell therapy to treat brain or spinal cord injury can depend upon the location of the damage being treated. The replacement cells can be stem cells (e.g., induced pluripotent stem cells), cells produced from stem cells, or cells obtained from a donor. The replacement cells can be glial progenitor cells, neural progenitor cells, or a combination thereof. The replacement cells can be differentiated cells that would normally be found in the damaged region of the brain or spinal cord. The replacement cells can be bone marrow stem cells. Cells and immune modulating adjuvants can be administered in subjects with a brain or spinal cord injury by injection or infusion into a brain region, the spinal cord, or the spinal canal.

Heart Disease

A subject treated with the cell therapy compositions and methods disclosed herein can have a heart disease. Cell therapy can be useful in treating heart disease at least because heart disease can result in the replacement of contractile cardiomyocytes with scar tissue. Replacement cells used to treat subjects with heart disease can be stem cells (e.g., induced pluripotent stem cells), cells produced from stem cells, or cells obtained from a donor. The replacement cells can comprise skeletal myoblasts, bone marrow-derived cells, cardiac stem cells, mesenchymal stem cells, contractile cardiomyocytes, cardiac progenitors, endothelial cells, smooth muscle cells, or a combination thereof. In some examples, the replacement cells can be administered as cell sheets. Cells and immune modulating adjuvants can be administered in subjects with heart disease by, for example, intravascular infusion (e.g., intravenous, intracoronary, or retrograde coronary sinus infusion), intramyocardial injection (e.g., transendocardial catheter-based injection, epicardial injection), or by scaffold or patch-based epicardial delivery to the myocardium.

Liver Disease

A subject treated with the cell therapy compositions and methods disclosed herein can have a liver disease. Subjects with either acute liver failure, chronic liver disease, or liver-based inborn errors of metabolism can be treated by cell therapy. Replacement cells used to treat subjects with liver disease can be stem cells (e.g., induced pluripotent stem cells), cells produced from stem cells, or cells obtained from a donor. The replacement cells can comprise hepatocytes, Cells and immune modulating adjuvants can be administered in subjects with liver disease by, for example, infusion or injection through the portal vein into the liver, by direct injection into an extrahepatic site (e.g., the spleen, lymph node), or large scale transplantation of cells into a de-cellularized liver scaffold.

Baldness

A subject treated with the cell therapy compositions and methods disclosed herein can be suffering from baldness. Hair follicles naturally contain stem cells, although follicle cells can shrink during the aging process, Replacement cells used to treat subjects for baldness can be stem cells (e.g., induced pluripotent stem cells), cells produced from stem cells, or cells obtained from a donor. The replacement cells can include follicle cells.

Missing Teeth

A subject treated with the cell therapy compositions and methods disclosed herein can be in need of replacement teeth. Replacement cells used to treat subjects in need of replacement teeth can be stem cells (e.g., induced pluripotent stem cells), cells produced from stem cells, or cells obtained from a donor. The replacement cells can be a tooth bud, which can develop into a new tooth when implanted into the gums.

Hearing Impairment

A subject treated with the cell therapy compositions and methods disclosed herein can have hearing impairment (e.g., hearing loss, tinitis, vertigo, instability or loss of balance, nausea, or a combination thereof). The hearing impairment can be caused by, for example, an ototoxic chemical, radiation, noise, age, or a combination thereof. Replacement cells used in cell therapy to treat hearing impairment can stem cells (e.g., induced pluripotent stem cells), cells produced from stem cells, or cells obtained from a donor. The replacement cells can include hair cells.

Cells and immune modulating adjuvants can be administered to the inner ear in subjects with hearing impairment by a variety of techniques, including intratympanic administration, intracochlear administration, transtympanic injection, or a combination thereof. These include the use of devices to transport and/or deliver the cells and immune modulating adjuvants in a targeted fashion to the membranes of the round or oval window, where it diffuses into the inner ear or is actively infused. Examples are otowicks (see, e.g., U.S. Pat. No. 6,120,484 to Silverstein), round window catheters (see, e.g., U.S. Pat. Nos. 5,421,818; 5,474,529; 5,476,446; 6,045,528; 6,377,849), or microimplants (see, e.g., WO2004/064912). They further include the use of devices that are inserted into the cochlear duct or any other part of the cochlea (see, e.g., U.S. Pat. No. 6,309,410). Another delivery technique is transtympanic injection (sometimes also called “intratympanic injection”), whereas the cells and immune modulating adjuvants are injected through the tympanic membrane into the middle ear typically for diffusion across the round window membrane (for a description, see, e.g., Light J. and Silverstein H., Current Opinion in Otolaryngology & Head and Neck Surgery (12): 378-383 (2004). For repeated injections, a middle ear ventilation tube may be inserted into the tympanic membrane, through which the cells and immune modulating adjuvants can be administered into the middle ear space.

Retinal Diseases

A subject treated with the cell therapy compositions and methods disclosed herein can have a retinal disease. For example, the subject can have macular degeneration, diabetic eye disease, age-related macular degeneration, branch retinal vein occlusion, central retinal vein occlusion, central retinal artery occlusion, central serous retinopathy, diabetic retinopathy, Fuchs' dystrophy, giant cell arteritis, glaucoma, hypertensive retinopathy, thyroid eye disease, iridocorneal endothelial syndrome, ischemic optic neuropathy, juvenile macular degeneration, macular edema, macular telangioctasia, marfan syndrome, optic neuritis, photokeratitis, retinitis pigmentosa, retinopathy of prematurity, stargardt disease, usher syndrome, Wolfram syndrome, Leber Congenital Amaurosis, or any other retinal disease. The subject can suffer from impaired vision, night blindness, retinal detachment, light sensitivity, tunnel vision, loss of peripheral vision, blindness, spots, or a combination thereof.

Replacement cells used in cell therapy to treat retinal diseases can include stem cells (e.g., induced pluripotent stem cells), cells produced from stem cells, or cells obtained from a donor. The replacement cells can include retinal pigment epithelium cells, corneal stem cells, retinal photoreceptor cells, retinal ganglion cells, bipolar cells, horizontal cells, amacrine cells, a combination thereof. The replacement cells can be sheets of retinal pigment epithelium cells.

Cells and immune modulating adjuvants can be administered in subjects with retinal diseases by, for example, intravitreal administration, intracameral administration, systemic administration, conjunctival administration, intracorneal administration, intraocular administration, ophthalmic administration, retrobulbar administration, subconjunctival administration, or by transplant.

Muscular Dystrophies

A subject treated with the cell therapy compositions and methods disclosed herein can have a muscular dystrophy (e.g., Duchenne Muscular Dystrophy). Replacement cells used in cell therapy to treat muscular dystrophy can include stem cells (e.g., induced pluripotent stem cells), cells produced from stein cells, or cells obtained from a donor. Replacement cells can include satellite cells, myoblasts, bone marrow cells, blood vessel cells, skeletal myogenic progenitors, or a combination thereof. Cells and immune modulating adjuvants can be administered in subjects with muscular dystrophy by intramuscular injection or systemic injection.

Diabetes

A subject treated with the cell therapy compositions and methods disclosed herein can have diabetes. Replacement cells used in cell therapy to treat diabetes can include stem cells (e.g., induced pluripotent stem cells), cells produced from stem cells, or cells obtained from a donor. Replacement cells can include pancreatic islet cells (e.g., alpha cells, beta cells, delta cells, P cells, epsilon cells). In some embodiments, the replacement cells are pancreatic beta cells. The pancreatic beta cells can be aggregates or dissociated cells. Replacement cells can be intact pancreatic islets. Cells and immune modulating adjuvants can be administered in subjects with diabetes, for example, by implantation in the liver. Implantation in the liver can be accomplished by transcutaneous catheter infusion through the liver into the portal vein. Cells and immune modulating adjuvants can be administered in subjects with diabetes, for example, by implantation in the pancreas.

Wounds

The cell therapy compositions and methods disclosed herein can be used to improve wound healing. The wounds can be acute wounds or slow healing or chronic wounds. The use of cell therapy to improve wound healing can be beneficial because it can minimize scar formation. Replacement cells used in cell therapy to improve wound healing can include stem cells (e.g., induced pluripotent stem cells), produced from stem cells, or obtained from a donor. Cells and immune modulating adjuvants can be administered directly to a wound site in subjects in order to improve wound healing.

MANF Family Proteins

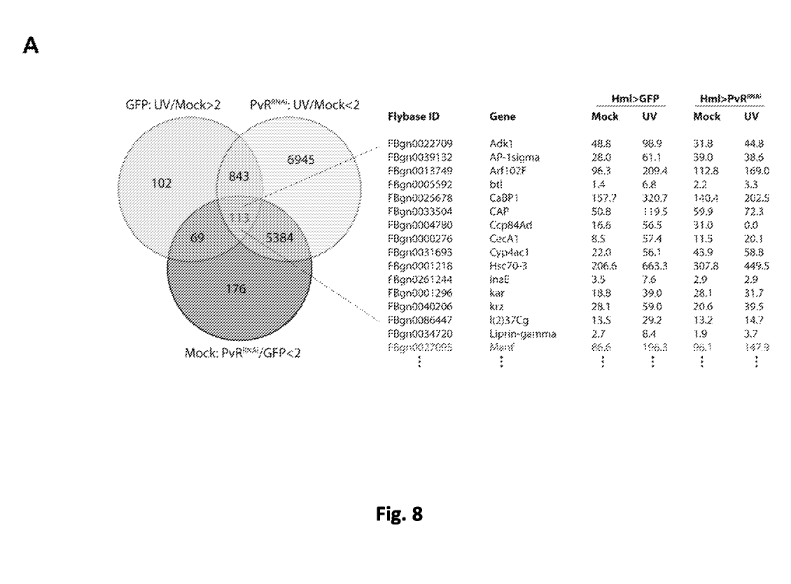

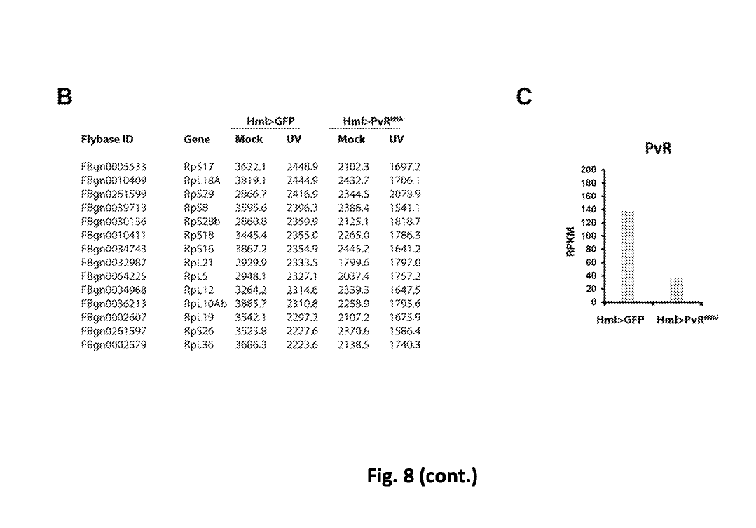

Mesencephalic Astrocyte-derived Neurotrophic Factor (MANF) was identified in a genetic screen for factors that may promote tissue repair and regeneration. FIG. 8A. MANF is a member of a family of neurotrophic factors (MANF family) that also includes conserved dopamine neurotrophic factors. MANF family proteins have been shown to exert a direct neuroprotective effect both in vitro and in vivo, and can reduce cell stress related apoptosis. Surprisingly, the data presented herein shows that MANF can also modulate the immune system. Accordingly, in one aspect of the invention, MANF can be used by itself or in combination with other treatment options, e.g., cell therapy.

MANF can be used by itself to address a certain sub-population of individuals that have or are suspected to have environmentally caused retinal injuries and/or degeneration (as opposed to injuries and/or degeneration that arise from inherited or genetic causes). In some aspects, these individuals do not have the inherited or genetic retinal diseases.

MANF can be used by itself to treat a certain sub-population of individuals that have or are suspected to have inflammation in a microenvironment. In one non-limiting embodiment, the retinal area is the microenvironment. In some aspects, the sub-population of individuals have or suspected of having a retinal injury and/or degeneration also have some form of inflammation in the retinal microenvironment. In other aspects, MANF can be used by itself to treat a certain sub-population of individuals that have or are suspected to have one or more (or a combination thereof) of the indications as listed herein.

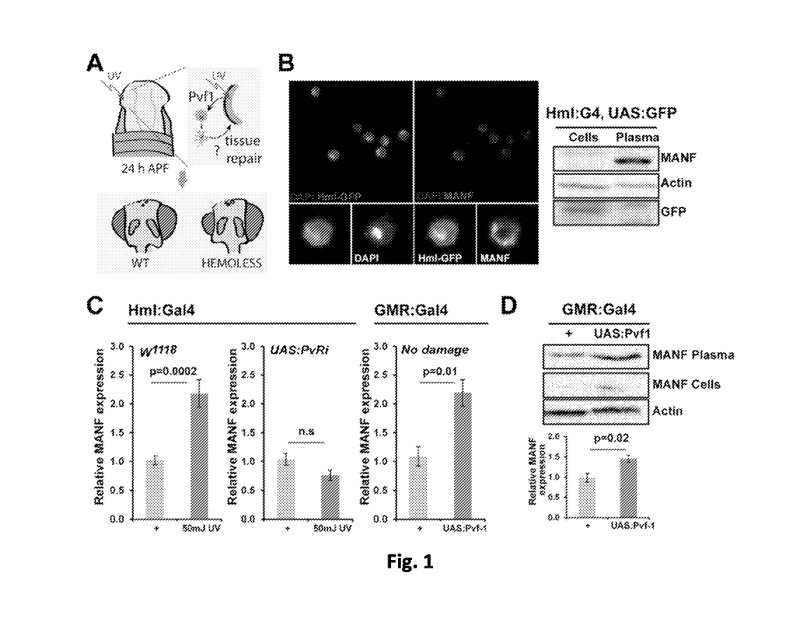

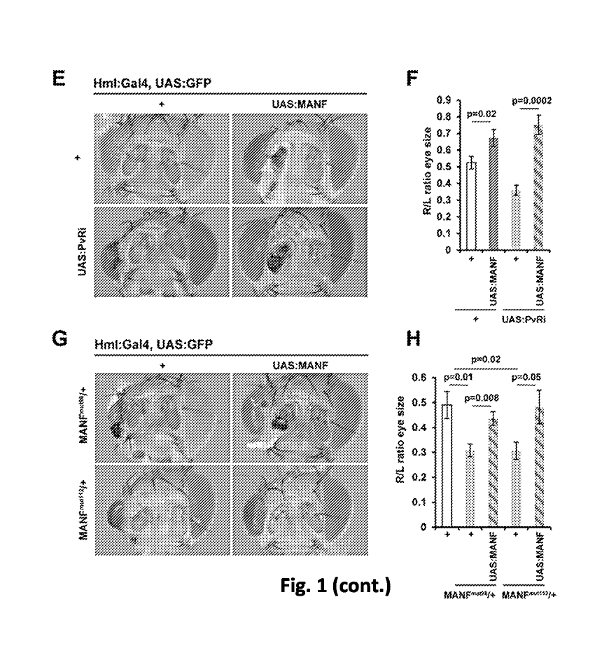

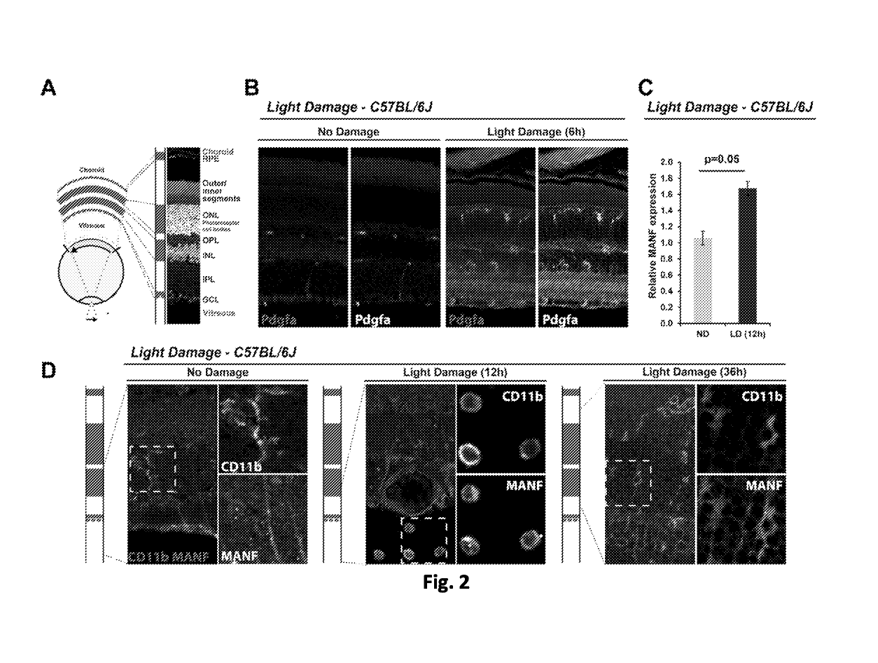

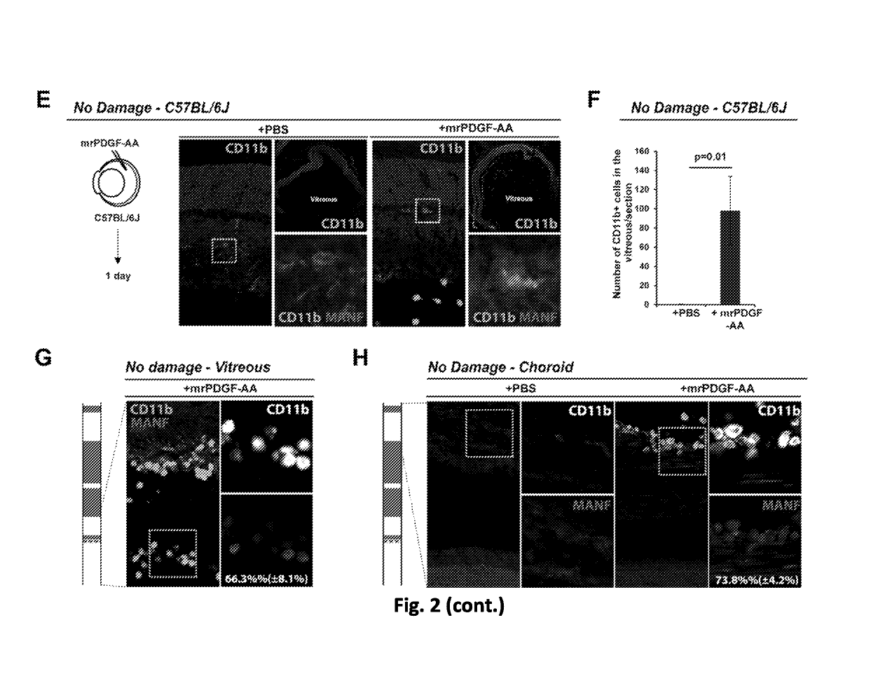

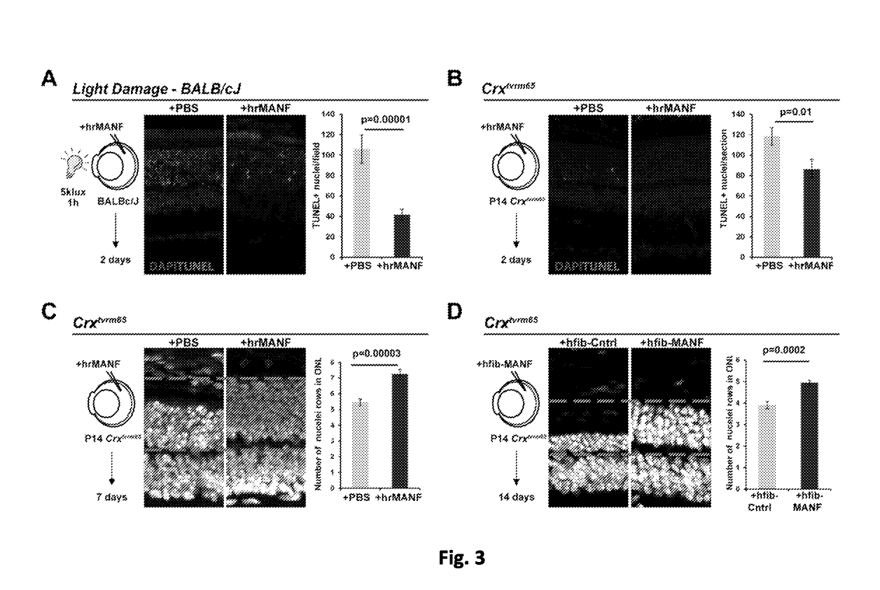

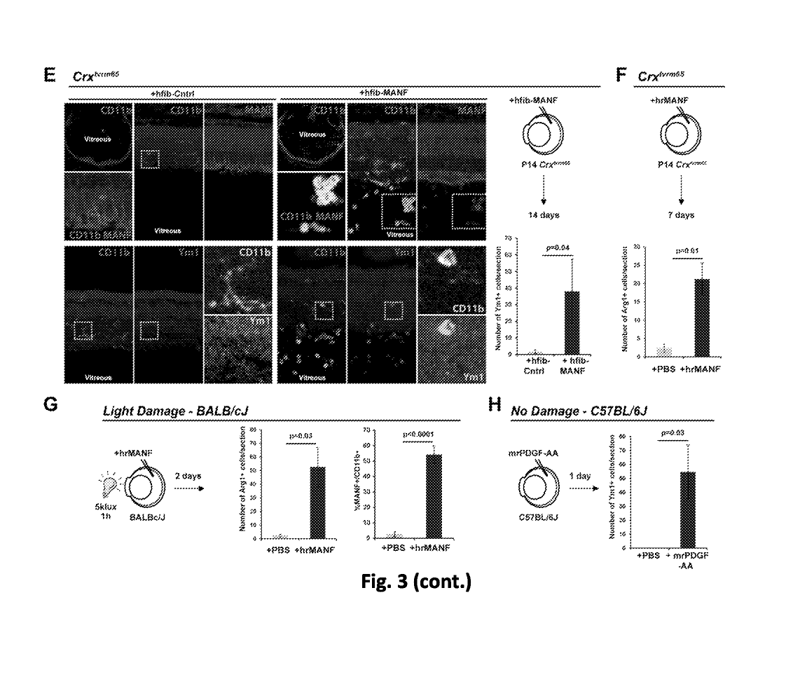

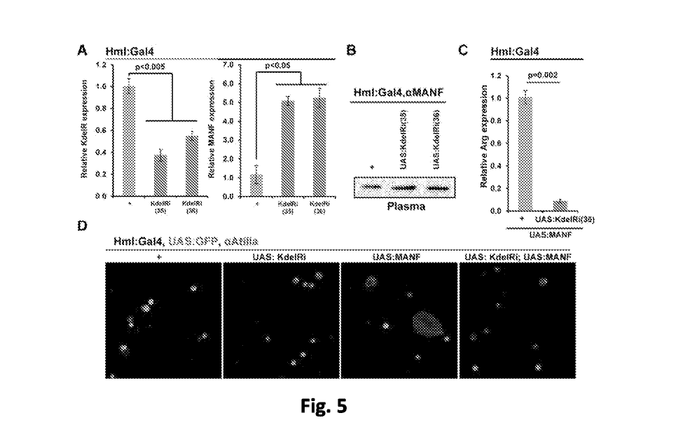

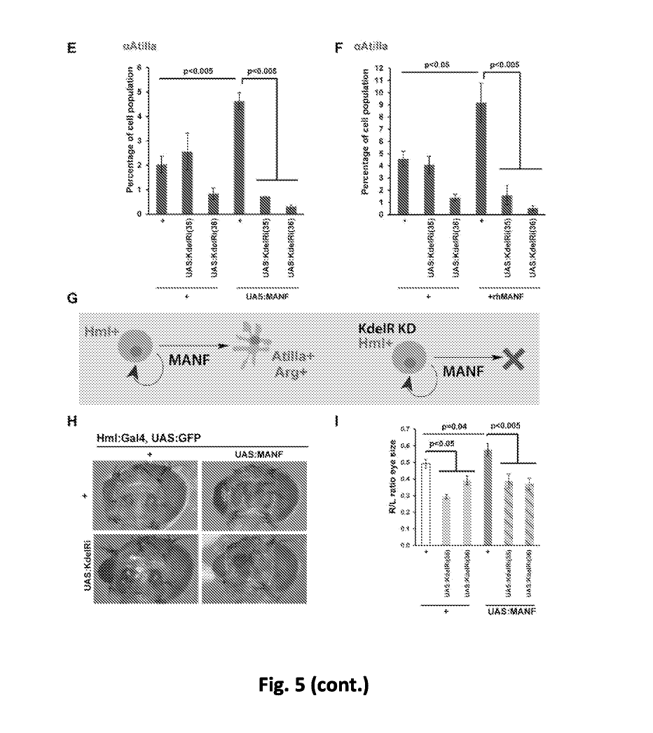

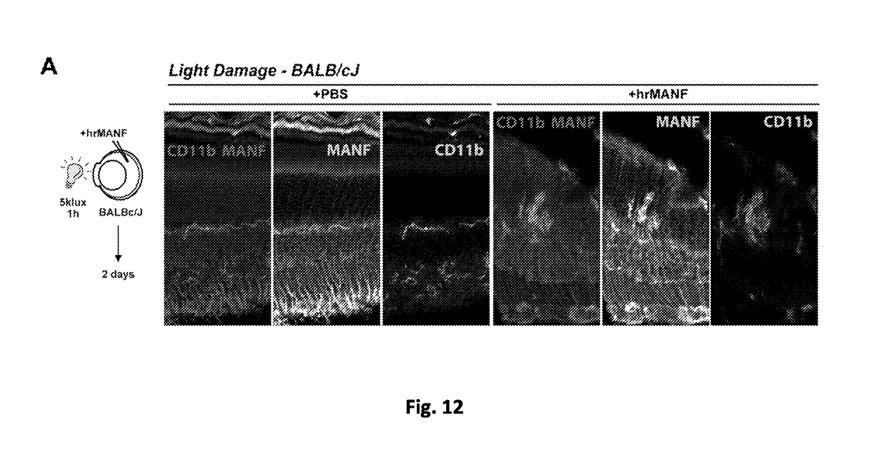

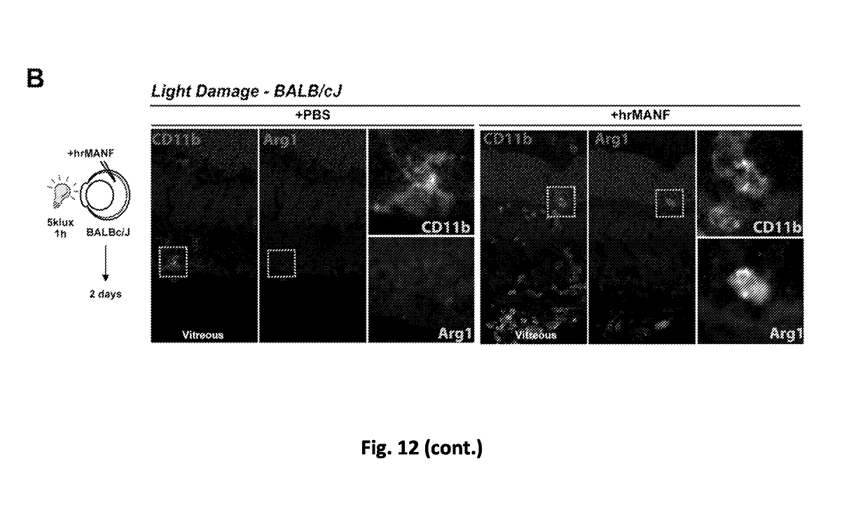

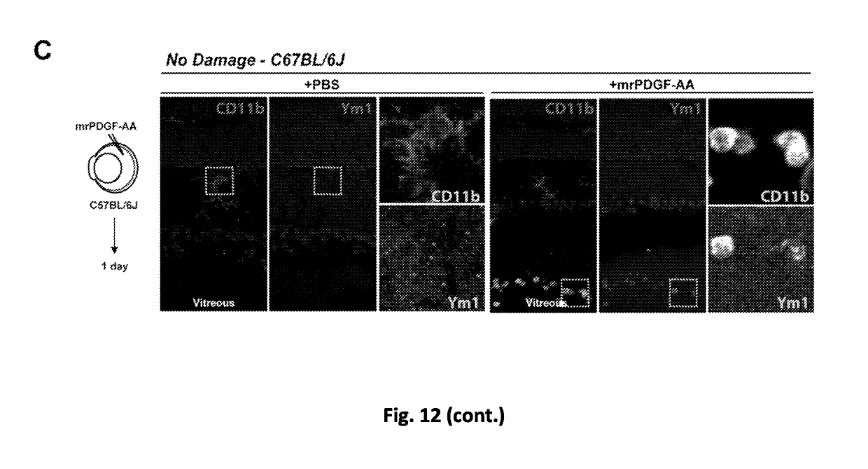

The experiments presented herein show that MANF is expressed in immune cells and that this expression is induced by tissue damage (FIGS. 1 and 2). Indeed, exogenous MANF increased recruitment of MANF expressing innate immune cells in damaged retina (FIGS. 3 and 12). Importantly, these immune cells also expressed markers of M2 activation, which is associated with resolution of inflammation and tissue repair. Interestingly, the data indicate that immune cell activation by MANF requires interaction with Kdel receptors (FIG. 5). Both MANF and CDNF contain c-terminal amino acid sequences (RTDL and KTEL respectively) that are conservative substitutions of the consensus KDEL signal that binds to Kdel receptors.

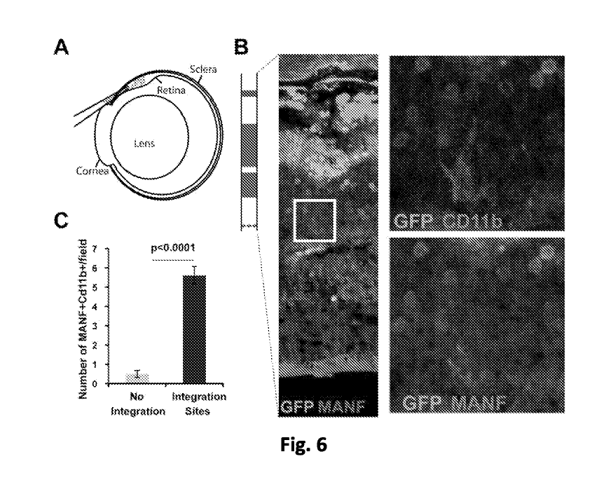

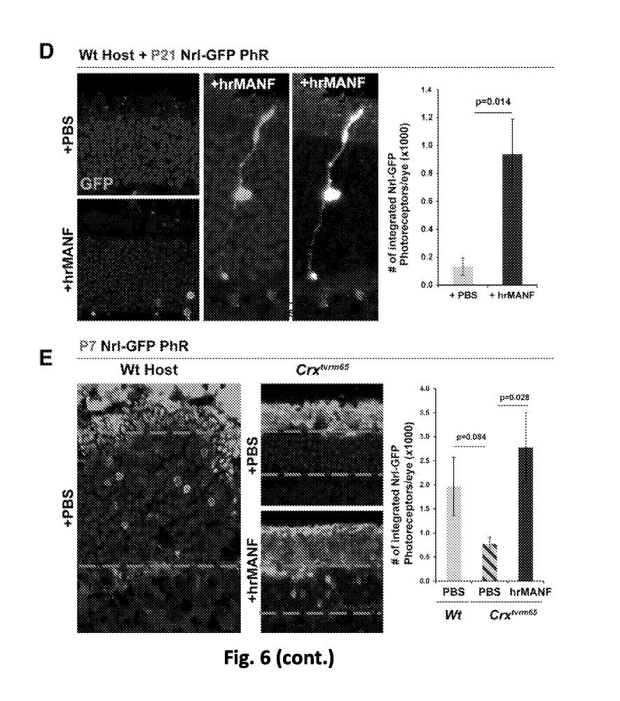

Overexpression or injection of MANF reduced tissue loss in multiple models of retinal degeneration. (FIGS. 1, 3, 9, 10, 11). Further, injection of MANF improved the integration efficiency of donor photoreceptor cells in replacement cell therapy (FIG. 6).

These findings support a role for MANF family proteins, and active fragments thereof, as adjuvants to improve cell therapy, both by improving the survival of transplanted cells, as well as in promoting a microenvironment supportive of local repair and integration of the transplanted cells. Accordingly, disclosed herein are immune modulating peptides comprising a MANF family protein or active fragment thereof, for use in the cell therapy methods and compositions disclosed herein. The immunomodulating peptides can be administered directly (e.g., by injection). The immunomodulating peptides can be delivered to a required site by administering one or more cells that secrete the immunomodulating peptides.

Immune Modulating Peptides

Immune modulating peptides can comprise amino acid sequences corresponding to a full length, or an active fragment of, a MANF family protein. The MANF family protein can be mesencephalic astrocytes-derived neurotrophic factor (MANF) or conserved dopamine neurotrophic factor (CDNF; also referred to as cerebral dopamine neurotrophic factor). In some embodiments, the immunomodulating peptide comprises MANF, or an active fragment thereof. Exemplary MANF peptide sequences can be found in Table 1. Exemplary MANF fragments can be found in Table 3. In some embodiments, the immunomodulating peptide comprises CDNF, or an active fragment thereof. Exemplary CDNF sequences can be found in Table 2. Exemplary CDNF fragments can be found in Table 4. A full length MANF or CDNF peptide sequence can be the pro-form, which contains a signal sequence, or the mature, secreted form in which the signal sequence is missing.

In some embodiments, the immunomodulating peptide comprises a protein having 70, 80, 85, 90, 95, 96, 97, 98, 99, or 100% homology (or identity) with the sequence of human CDNF or MANF protein.

In some embodiments, the immunomodulating peptide comprises about: 10%, 20%, 30%, 40%, 50%, 60%, 70%, 80%, 90%, or 100% of the length of human CDNF or MANF protein.

Immune modulating peptides can be a pro-form of MANF or an active fragment thereof. For example, the peptide sequence of the immunomodulating peptide can comprise or consist of a sequence that has at least about 80% identity with SEQ ID NO: 1. In another example, the peptide sequence of the immunomodulating peptide can comprise or consist of a sequence that has at least about 90% identity with SEQ ID NO: 1. In another example, the peptide sequence of the immunomodulating peptide can comprise or consist of a sequence that has at least about 95% identity with SEQ ID NO: 1. In another example, the peptide sequence of the immunomodulating peptide can comprise or consist of a sequence that has at least about 97% identity with SEQ ID NO: 1. In another example, the peptide sequence of the immunomodulating peptide can comprise or consist of a sequence that has 100% identity with SEQ ID NO: 1. In any of these examples, the immunomodulating peptide can have a length that is at least about 5% the length of SEQ ID NO: 1. In any of these examples, the immunomodulating peptide can have a length that is at least about 50% the length of SEQ ID NO: 1. In any of these examples, the immunomodulating peptide can have a length that is at least about 80% the length of SEQ ID NO: 1. In any of these examples, the immunomodulating peptide can have a length that is at least about 90% the length of SEQ ID NO: 1. In any of these examples, the immunomodulating peptide can have a length that is the same length as SEQ ID NO: 1. The immunomodulating peptide, in any of these examples can also have a maximum length. The maximum length can be, e.g., 100%, 90%, 80%, 70%, 60%, 50%, or 25% the length of SEQ ID NO: 1.

Immune modulating peptides can be a pro-form of MANF or an active fragment thereof. For example, the peptide sequence of the immunomodulating peptide can comprise or consist of a sequence that has at least about 80% identity with SEQ ID NO: 2. In another example, the peptide sequence of the immunomodulating peptide can comprise or consist of a sequence that has at least about 90% identity with SEQ ID NO: 2. In another example, the peptide sequence of the immune modulating peptide can comprise or consist of a sequence that has at least about 95% identity with SEQ ID NO: 2. In another example, the peptide sequence of the immune modulating peptide can comprise or consist of a sequence that has at least about 97% identity with SEQ ID NO: 2. In another example, the peptide sequence of the immune modulating peptide can comprise or consist of a sequence that has 100% identity with SEQ ID NO: 2. In any of these examples, the immune modulating peptide can have a length that is at least about 5% the length of SEQ ID NO: 2. In any of these examples, the immune modulating peptide can have a length that is at least about 50% the length of SEQ ID NO: 2. In any of these examples, the immune modulating peptide can have a length that is at least about 80% the length of SEQ ID NO: 2. In any of these examples, the immune modulating peptide can have a length that is at least about 90% the length of SEQ ID NO: 2. In any of these examples, the immune modulating peptide can have a length that is the same length as SEQ ID NO: 2. The immune modulating peptide, in any of these examples can also have a maximum length. The maximum length can be, e.g., 100%, 90%, 80%, 70%, 60%, 50%, or 25% the length of SEQ ID NO: 2.

Immune modulating peptides can be a mature or secreted form of MANF, or an active fragment thereof. For example, the peptide sequence of the immune modulating peptide can comprise or consist of a sequence that has at least about 80% identity with SEQ ID NO: 3. In another example, the peptide sequence of the immune modulating peptide can comprise or consist of a sequence that has at least about 90% identity with SEQ ID NO: 3. In another example, the peptide sequence of the immune modulating peptide can comprise or consist of a sequence that has at least about 95% identity with SEQ ID NO: 3. In another example, the peptide sequence of the immune modulating peptide can comprise or consist of a sequence that has at least about 97% identity with SEQ ID NO: 3. In another example, the peptide sequence of the immune modulating peptide can comprise or consist of a sequence that has 100% identity with SEQ ID NO: 3. In any of these examples, the immune modulating peptide can have a length that is at least about 5% the length of SEQ ID NO: 3. In any of these examples, the immune modulating peptide can have a length that is at least about 50% the length of SEQ ID NO: 3. In any of these examples, the immune modulating peptide can have a length that is at least about 80% the length of SEQ ID NO: 3. In any of these examples, the immune modulating peptide can have a length that is at least about 90% the length of SEQ ID NO: 3. In any of these examples, the immune modulating peptide can have a length that is the same length as SEQ ID NO: 3. The immune modulating peptide, in any of these examples can also have a maximum length. The maximum length can be, e.g., 100%, 90%, 80%, 70%, 60%, 50%, or 25% the length of SEQ ID NO: 3.

Immune modulating peptides can be a synthetic form of MANF, or an active fragment thereof. The synthetic form of MANF contains a non-natural N-terminal methionine. The N-terminal methionine can enable production of the synthetic form of MANF in cell lines lacking the post-translational modification machinery to process the pro-form of MANF to the secreted or mature form of MANF. For example, the peptide sequence of the immune modulating peptide can comprise or consist of a sequence that has at least about 80% identity with SEQ ID NO: 4. In another example, the peptide sequence of the immune modulating peptide can comprise or consist of a sequence that has at least about 90% identity with SEQ ID NO: 4. In another example, the peptide sequence of the immune modulating peptide can comprise or consist of a sequence that has at least about 95% identity with SEQ ID NO: 4. In another example, the peptide sequence of the immune modulating peptide can comprise or consist of a sequence that has at least about 97% identity with SEQ ID NO: 4. In another example, the peptide sequence of the immune modulating peptide can comprise or consist of a sequence that has 100% identity with SEQ ID NO: 4. In any of these examples, the immune modulating peptide can have a length that is at least about 5% the length of SEQ ID NO: 4. In any of these examples, the immune modulating peptide can have a length that is at least about 50% the length of SEQ ID NO: 4. In any of these examples, the immune modulating peptide can have a length that is at least about 80% the length of SEQ ID NO: 4. In any of these examples, the immune modulating peptide can have a length that is at least about 90% the length of SEQ ID NO: 4. In any of these examples, the immune modulating peptide can have a length that is the same length as SEQ ID NO: 4. The immune modulating peptide, in any of these examples can also have a maximum length. The maximum length can be, e.g., 100%, 90%, 80%, 70%, 60%, 50%, or 25% the length of SEQ ID NO: 4.

TABLE 1
Human MANF Protein Sequences

| SEQ | | ASCESSION | |
|---|---|---|---|
| ID | NAME | Number | SEQUENCE |
| SEQ | Human Pro- | NP_006001 | MRRMRRMWAT QGLAVALALS VLPGSRALRP |
| ID | MANF | | GDCEVCISYL GRFYQDLKDR DVTFSPATIE |
| NO: 1 | | | NELIKFCREA RGKENRLCYY IGATDDAATK |
| | | | IINEVSKPLA HHIPVEKICE KLKKKDSQIC |
| | | | ELKYDKQIDL STVDLKKLRV KELKKILDDW |
| | | | GETCKGCAEK SDYIRKINEL MPKYAPKAAS |
| | | | ART DL |
| SEQ | Human Pro- | | MWATQGLAVA LALSVLPGSR ALRPGDCEVC |
| ID | MANF | | ISYLGRFYQD LKDRDVTFSP ATIENELIKF |
| NO: 2 | | | CREARGKENR LCYYIGATDD AATKIINEVS |
| | | | KPLAHHIPVE KICEKLKKKD SQICELKYDK |
| | | | QIDLSTVDLK KLRVKELKKI LDDWGETCKG |
| | | | CAEKSDYIRK INELMPKYAP KAASARTDL |
| SEQ | Human MANF | | LRPGDCEVCI SYLGRFYQDL KDRDVTFSPA |
| ID | (Secreted | | TIENELIKFC REARGKENRL CYYIGATDDA |
| NO: 3 | Form) | | ATKIINEVSK PLAHHIPVEK ICEKLKKKDS |
| | | | QICELKYDKQ IDLSTVDLKK LRVKELKKIL |
| | | | DDWGETCKGC AEKSDYIRKI NELMPKYAPK |
| | | | AASARTDL |
| SEQ | Human | | MLRPGDCEVC ISYLGRFYQD LKDRDVTFSP |
| ID | Synthetic | | ATIENELIKF CREARGKENR LCYYIGATDD |
| NO: 4 | MANF | | AATKIINEVS KPLAHHIPVE KICEKLKKKD |
| | | | SQICELKYDK QIDLSTVDLK KLRVKELKKI |
| | | | LDDWGETCKG CAEKSDYIRK INELMPKYAP |
| | | | KAASARTDL |

Immune modulating peptides can be a pro-form of CDNF, or an active fragment thereof. For example, the peptide sequence of the immune modulating peptide can comprise or consist of a sequence that has at least about 80% identity with SEQ ID NO: 5. In another example, the peptide sequence of the immune modulating peptide can comprise or consist of a sequence that has at least about 90% identity with SEQ ID NO: 5. In another example, the peptide sequence of the immune modulating peptide can comprise or consist of a sequence that has at least about 95% identity with SEQ ID NO: 5. In another example, the peptide sequence of the immune modulating peptide can comprise or consist of a sequence that has at least about 97% identity with SEQ ID NO: 5. In another example, the peptide sequence of the immune modulating peptide can comprise or consist of a sequence that has 100% identity with SEQ ID NO: 5. In any of these examples, the immune modulating peptide can have a length that is at least about 5% the length of SEQ ID NO: 5. In any of these examples, the immune modulating peptide can have a length that is at least about 50% the length of SEQ ID NO: 5. In any of these examples, the immune modulating peptide can have a length that is at least about 80% the length of SEQ ID NO: 5. In any of these examples, the immune modulating peptide can have a length that is at least about 90% the length of SEQ ID NO: 5. In any of these examples, the immune modulating peptide can have a length that is the same length as SEQ ID NO: 5. The immune modulating peptide, in any of these examples can also have a maximum length. The maximum length can be, e.g., 100%, 90%, 80%, 70%, 60%, 50%, or 25% the length of SEQ ID NO: 5.

Immune modulating peptides can be a mature or secreted form of CDNF, or an active fragment thereof. For example, the peptide sequence of the immune modulating peptide can comprise or consist of a sequence that has at least about 80% identity with SEQ ID NO: 6. In another example, the peptide sequence of the immune modulating peptide can comprise or consist of a sequence that has at least about 90% identity with SEQ ID NO: 6. In another example, the peptide sequence of the immune modulating peptide can comprise or consist of a sequence that has at least about 95% identity with SEQ ID NO: 6. In another example, the peptide sequence of the immune modulating peptide can comprise or consist of a sequence that has at least about 97% identity with SEQ ID NO: 6. In another example, the peptide sequence of the immune modulating peptide can comprise or consist of a sequence that has 100% identity with SEQ ID NO: 6. In any of these examples, the immune modulating peptide can have a length that is at least about 5% the length of SEQ ID NO: 6. In any of these examples, the immune modulating peptide can have a length that is at least about 50% the length of SEQ ID NO: 6. In any of these examples, the immune modulating peptide can have a length that is at least about 80% the length of SEQ ID NO: 6. In any of these examples, the immune modulating peptide can have a length that is at least about 90% the length of SEQ ID NO: 6. In any of these examples, the immune modulating peptide can have a length that is the same length as SEQ ID NO: 6. The immune modulating peptide, in any of these examples can also have a maximum length. The maximum length can be, e.g., 100%, 90%, 80%, 70%, 60%, 50%, or 25% the length of SEQ ID NO: 6.

Immune modulating peptides can be a synthetic form of CDNF, or an active fragment thereof. The synthetic form of CDNF contains a non-natural N-terminal methionine. The N-terminal methionine can enable production of the synthetic form of CDNF in cell lines lacking the post-translational modification machinery to process the pro-form of CDNF to the secreted or mature form of CDNF. For example, the peptide sequence of the immune modulating peptide can comprise or consist of a sequence that has at least about 80% identity with SEQ ID NO: 7. In another example, the peptide sequence of the immune modulating peptide can comprise or consist of a sequence that has at least about 90% identity with SEQ ID NO: 7. In another example, the peptide sequence of the immune modulating peptide can comprise or consist of a sequence that has at least about 95% identity with SEQ ID NO: 7. In another example, the peptide sequence of the immune modulating peptide can comprise or consist of a sequence that has at least about 97% identity with SEQ ID NO: 7. In another example, the peptide sequence of the immune modulating peptide can comprise or consist of a sequence that has 100% identity with SEQ ID NO: 7. In any of these examples, the immune modulating peptide can have a length that is at least about 5% the length of SEQ ID NO: 7. In any of these examples, the immune modulating peptide can have a length that is at least about 50% the length of SEQ ID NO: 7. In any of these examples, the immune modulating peptide can have a length that is at least about 80% the length of SEQ ID NO: 7. In any of these examples, the immune modulating peptide can have a length that is at least about 90% the length of SEQ ID NO: 7. In any of these examples, the immune modulating peptide can have a length that is the same length as SEQ ID NO: 7. The immune modulating peptide, in any of these examples can also have a maximum length. The maximum length can be, e.g., 100%, 90%, 80%, 70%, 60%, 50%, or 25% the length of SEQ ID NO: 7.

TABLE 2
Human CDNF Protein Sequences

| SEQ | | ASCESSION | |
|---|---|---|---|
| ID | NAME | Number | SEQUENCE |
| SEQ | Human CDNF | NP_001025125 | MWCASPVAVV AFCAGLLVSH PVLTQGQEAG |
| ID | Precursor | | GRPGADCEVC KEFLNRFYKS LIDRGVNFSL |
| NO: 5 | | | DTIEKELISF CLDTKGKENR LCYYLGATKD |
| | | | AATKILSEVT RPMSVHMPAM KICEKLKKLD |
| | | | SQICELKYEK TLDLASVDLR KMRVAELKQI |
| | | | LHSWGEECRA CAEKTDYVNL IQELAPKYAA |
| | | | THPKTEL |
| SEQ | Human CDNF | | QEAGGRPGAD CEVCKEFLNR FYKSLIDRGV |
| ID | (Mature) | | NFSLDTIEKE LISFCLDTKG KENRLCYYLG |
| NO: 6 | | | ATKDAATKIL SEVTRPMSVH MPAMKICEKL |
| | | | KKLDSQICEL KYEKTLDLAS VDLRKMRVAE |
| | | | LKQILHSWGE ECRACAEKTD YVNLIQELAP |
| | | | KYAATHPKTE L |
| SEQ | Human | | MQEAGGRPGA DCEVCKEFLN RFYKSLIDRG |
| ID | Synthetic | | VNFSLDTIEK ELISFCLDTK GKENRLCYYL |
| NO: 7 | CDNF | | GATKDAATKI LSEVTRPMSV HMPAMKICEK |
| | | | LKKLDSQICE LKYEKTLDLA SVDLRKMRVA |
| | | | ELKQILHSWG EECRACAEKT DYVNLIQELA |
| | | | PKYAATHPKT EL |

Active fragments of MANF or CDNF can include short peptides with a length of about 4-40 amino acids; for example, about: 4-40, 4-35, 4-30, 4-25, 4-20, 4-15, 4-10, 5-40, 6-40, 7-40, 8-40, 5-35, 5-30, 5-25, 5-20, 5-15, 5-10, 6-35, 6-30, 6-25, 6-20, 6-15, 6-10, 7-35, 7-30, 7-25, 7-20, 7-15, 7-10, 8-35, 8-30, 8-25, 8-20, or 8-15 amino acids. For example, the short peptides can consist of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. The peptides may comprise any of the naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine as well as non-conventional or modified amino acids. The peptide can have 70, 80, 85, 90, 95, 96, 97, 98, 99, or 100% homology (or identity) with the sequence of human CDNF or MANF protein.

CKGC (SEQ ID NO:32) and CRAC (SEQ ID NO:34) have been identified as evolutionarily conserved sequences in MANF and CDNF respectively. (see, e.g., WO 2013/034805) In some embodiments, the short peptides comprise the sequence CXXC. In some embodiments, the peptides comprise the sequence CKGC or CRAC.

The date presented herein identify the KDEL like c-terminal sequences of MANF and CDNF as important for immune cell activation. In some embodiments, the short peptides comprise the sequence RTDL (SEQ ID NO:33) or KTEL (SEQ ID NO:35).

In some embodiments, the short peptides comprise both the CXXC motif and the KDEL-like sequence of either MANF or CDNF. The CXXC motif and the KDEL-like sequence can be separated, for example, by from 0 to 32 amino acids. For example, the CXXC motif and the KDEL-like sequence can be separated by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 amino acids. In some embodiments, the immune modulating peptide comprises the sequences CKGC and RTDL. separated by from 0 to 24 amino acids. In some embodiments, the peptides comprise the sequences CRAC and KTEL separated by from 0 to 22 amino acids.

These short peptides disclosed herein can be cell permeable. Active fragments of MANF can include any of the short peptides disclosed in Table 3. Active fragments of CDNF can include any of the short peptides disclosed in Table 4.

TABLE 3
Short peptides of human MANF.

| SEQ ID NO | SEQUENCE |
|---|---|
| SEQ ID | ILDDWGETCKGCAEKSDYIRKINELMPKYAPKAASARTDL |
| NO: 8 | |
| SEQ ID | LDDWGETCKGCAEKSDYIRKINELMPKYAPKAASARTDL |
| NO: 9 | |
| SEQ ID | DDWGETCKGCAEKSDYIRKINELMPKYAPKAASARTDL |
| NO: 10 | |
| SEQ ID | DWGETCKGCAEKSDYIRKINELMPKYAPKAASARTDL |
| NO: 11 | |
| SEQ ID | WGETCKGCAEKSDYIRKINELMPKYAPKAASARTDL |
| NO: 12 | |
| SEQ ID | GETCKGCAEKSDYIRKINELMPKYAPKAASARTDL |
| NO: 13 | |
| SEQ ID | EICKGCAEKSDYIRKINELMPKYAPKAASARIDL |
| NO: 14 | |
| SEQ ID | ICKGCAEKSDYIRKINELMPKYAPKAASARIDL |
| NO: 15 | |
| SEQ ID | CKGCAEKSDYIRKINELMPKYAPKAASARTDL |
| NO: 16 | |

TABLE 4
Short peptides of human CDNF.

| SEQ ID NO | SEQUENCE |
|---|---|
| SEQ ID | KQILHSWGEECRACAEKTDYVNLIQELAPKYAATHPKTEL |
| NO: 17 | |
| SEQ ID | QILHSWGEECRACAEKTDYVNLIQELAPKYAATHPKTEL |
| NO: 18 | |
| SEQ ID | ILHSWGEECRACAEKTDYVNLIQELAPKYAATHPKTEL |
| NO: 19 | |
| SEQ ID | LHSWGEECRACAEKTDYVNLIQELAPKYAATHPKTEL |
| NO: 20 | |
| SEQ ID | HSWGEECRACAEKTDYVNLIQELAPKYAATHPKTEL |
| NO: 21 | |
| SEQ ID | SWGEECRACAEKTDYVNLIQELAPKYAATHPKTEL |
| NO: 22 | |
| SEQ ID | WGEECRACAEKTDYVNLIQELAPKYAATHPKTEL |
| NO: 23 | |
| SEQ ID | GEECRACAEKTDYVNLIQELAPKYAATHPKTEL |
| NO: 24 | |
| SEQ ID | EECRACAEKTDYVNLIQELAPKYAATHPKTEL |
| NO: 25 | |
| SEQ ID | ECRACAEKTDYVNLIQELAPKYAATHPKTEL |
| NO: 26 | |
| SEQ ID | CRACAEKTDYVNLIQELAPKYAATHPKTEL |
| NO: 27 | |

Immune modulating peptides can be conjugated to a detectable chemical or biochemical moiety such as a FITC-label or an Alexa probe. As used herein, a “detectable chemical or biochemical moiety” means a tag that exhibits an amino acid sequence or a detectable chemical or biochemical moiety for the purpose of facilitating detection of the peptide; such as a detectable molecule selected from among: a visible, fluorescent, chemiluminescent, or other detectable dye; an enzyme that is detectable in the presence of a substrate, e.g., an alkaline phosphatase with NBT plus BCIP or a peroxidase with a suitable substrate; a detectable protein, e.g., a green fluorescent protein. Preferably, the tag does not prevent or hinder the penetration of the peptide into the target cell.

Effective Amounts

With respect to immune modulating peptides disclosed herein, (e.g., MANF family proteins or active fragments thereof), the effective amount can vary, as described herein.

The effective amount of an immune modulating peptide (e.g., MANF family proteins or active fragments thereof, e.g., MANF, CDNF, or fragments thereof) can expressed as a weight of the immune modulating peptide. The weights disclosed should be interpreted as an amount that is administered in a single dose to a site of administration. For example, the effective amount of the immune modulating peptide can be about: 0.5 μg-2.5 μg, 0.5 μg-5 μg, 0.5 μg-7.5 μg, 0.5 μg-12.5 μg, 0.5 μg-25 μg, 0.5 μg-50 μg, 0.5 μg-75 μg, 0.5 μg-100 μg, 0.5 μg-150 μg, 0.5 μg-250 μg, 0.5 μg-500 μg, 0.5 μg-1000 μg, 0.5 μg-1250 μg, 0.5 μg-2500 μg, 1 μg-2.5 μg, 1 μg-5 μg, 1 μg-7.5 μg, 1 μg-12.5 μg, 1 μg-25 μg, 1 μg-50 μg, 1 μg-75 μg, 1 μg-100 μg, 1 μg-150 μg, 1 μg-250 μg, 1 μg-500 μg, 1 μg-1000 μg, 1 μg-1250 μg, 1 μg-2500 μg, 2.5 μg-5 μg, 2.5 μg-7.5 μg, 2.5 μg-12.5 μg, 2.5 μg-25 μg, 2.5 μg-50 μg, 2.5 μg-75 μg, 2.5 μg-100 μg, 2.5 μg-150 μg, 2.5 μg-250 μg, 2.5 μg-500 μg, 2.5 μg-1000 μg, 2.5 μg-1250 μg, 2.5 μg-2500 μg, 5 μg-7.5 μg, 5 μg-12.5 μg, 5 μg-25 μg, 5 μg-50 μg, 5 μg-75 μg, 5 μg-100 μg, 5 μg-150 μg, 5 μg-250 μg, 5 μg-500 μg, 5 μg-1000 μg, 5 μg-1250 μg, 5 μg-2500 μg, 7.5 μg-12.5 μg, 7.5 μg-25 μg, 7.5 μg-50 μg, 7.5 μg-75 μg, 7.5 μg-100 μg, 7.5 μg-150 μg, 7.5 μg-250 μg, 7.5 μg-500 μg, 7.5 μg-1000 μg, 7.5 μg-1250 μg, 7.5 μg-2500 μg, 12.5 μg-25 μg, 12.5 μg-50 μg, 12.5 μg-75 μg, 12.5 μg-100 μg, 12.5 μg-150 μg, 12.5 μg-250 μg, 12.5 μg-500 μg, 12.5 μg-1000 μg, 12.5 μg-1250 μg, 12.5 μg-2500 μg, 25 μg-50 μg, 25 μg-75 μg, 25 μg-100 μg, 25 μg-150 μg, 25 μg-250 μg, 25 μg-500 μg, 25 μg-1000 μg, 25 μg-1250 μg, 25 μg-2500 μg, 50 μg-75 μg, 50 μg-100 μg, 50 μg-150 μg, 50 μg-250 μg, 50 μg-500 μg, 50 μg-1000 μg, 50 μg-1250 μg, 50 μg-2500 μg, 75 μg-100 μg, 75 μg-150 μg, 75 μg-250 μg, 75 μg-500 μg, 75 μg-1000 μg, 75 μg-1250 μg, 75 μg-2500 μg, 100 μg-150 μg, 100 μg-250 μg, 100 μg-500 μg, 100 μg-1000 μg, 100 μg-1250 μg, 100 μg-2500 μg, 150 μg-250 μg, 150 μg-500 μg, 150 μg-1000 μg, 150 μg-1250 μg, 150 μg-2500 μg, 250 μg-500 μg, 250 μg-1000 μg, 250 μg-1250 μg, 250 μg-2500 μg, 500 μg-1000 μg, 500 μg-1250 μg, 500 μg-2500 μg, 1000 μg-1250 μg, 1000 μg-2500 μg, or 1250 μg-2500 μg. The effective amount can be about 1 μg-500 μg. The effective amount can be about 5 μg-250 μg.

The effective amount of an immune modulating peptide (e.g., MANF family proteins or active fragments thereof, e.g., MANF, CDNF, or fragments thereof) can be at least about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg.

The effective amount of an immune modulating peptide (e.g., MANF family proteins or active fragments thereof, e.g., MANF, CDNF, or fragments thereof) can be less than about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg.

The effective amount of an immune modulating peptide (e.g., MANF family proteins or active fragments thereof, e.g., MANF, CDNF, or fragments thereof) can in a formulation having a wide range of concentrations. For example, the effective amount of the immune modulating peptide can be in a formulation at a concentration of about: 0.1 μMol-500 μMol, 0.1 μMol-100 μMol, 0.1 μMol-50 μMol, 0.1 μMol-20 μMol, 0.1 μMol-10 μMol, 0.1 μMol-5 μMol, 0.1 μMol-3 μMol, 0.1 μMol-1 μMol, 0.1 μMol-0.5 μMol, 0.5 μMol-500 μMol, 0.5 μMol-100 μMol, 0.5 μMol-50 μMol, 0.5 μMol-20 μMol, 0.5 μMol-10 μMol, 0.5 μMol-5 μMol, 0.5 μMol-3 μMol, 0.5 μMol-1 μMol, 1 μMol-500 μMol, 1 μMol-100 μMol, 1 μMol-50 μMol, 1 μMol-20 μMol, 1 μMol-10 μMol, 1 μMol-5 μMol, 1 μMol-3 μMol, 3 μMol-500 μMol, 3 μMol-100 μMol, 3 μMol-50 μMol, 3 μMol-20 μMol, 3 μMol-10 μMol, 3 μMol-5 μMol, 5 μMol-500 μMol, 5 μMol-100 μMol, 5 μMol-50 μMol, 5 μMol-20 μMol, 5 μMol-10 μMol, 10 μMol-500 μMol, 10 μMol-100 μMol, 10 μMol-50 μMol, 10 μMol-20 μMol, 20 μMol-500 μMol, 20 μMol-100 μMol, 20 μMol-50 μMol, 50 μMol-500 μMol, 50 μMol-100 μMol, or 100 μMol-500 μMol. In some examples, the effective amount of the immune modulating peptide is in a formulation at a concentration of about: 1 μMol-50 μMol. In some examples, the effective amount of the immune modulating peptide is in a formulation at a concentration of about: 3 μMol-20 μMol.

The effective amount of the immune modulating peptide in a formulation can be administered in a range of volumes. The range of volumes can depend upon the route of administration. The range of volumes can depend upon the site of administration.

For some sites or routes of administration, the range of volumes for a single dose can be from 1 μL, to about 1000 μL; for example, about: 1 μL-5 μL, 1 μL-25 μL, 1 μL-50 μL, 1 μL-100 μL, 1 μL-200 μL, 1 μL-250 μL, 1 μL-300 μL, 1 μL-500 μL, 1 μL-1000 μL, 5 μL-25 μL, 5 μL-50 μL, 5 μL-100 μL, 5 μL-200 μL, 5 μL-250 μL, 5 μL-300 μL, 5 μL-500 μL, 5 μL-1000 μL, 25 μL-50 μL, 25 μL-100 μL, 25 μL-200 μL, 25 μL-250 μL, 25 μL-300 μL, 25 μL-500 μL, 25 μL-1000 μL, 50 μL-100 μL, 50 μL-200 μL, 50 μL-250 μL, 50 μL-300 μL, 50 μL-500 μL, 50 μL-1000 μL, 100 μL-200 μL, 100 μL-250 μL, 100 μL-300 μL, 100 μL-500 μL, 100 μL-1000 μL, 200 μL-250 μL, 200 μL-300 μL, 200 μL-500 μL, 200 μL-1000 μL, 250 μL-500 μL, 250 μL-1000 μL, 300 μL-500 μL, 300 μL-1000, or 500 μL-1000 μL. The volume range can be about 50 μL-1000 μL. The volume range can be about 100 μL-500 μL. The volume range can be about 200 μL-300 μL. The volume range can be about 25 μL-100 μL.

Immune Modulating Peptide Delivery by Cells

Delivery of an immune modulating peptide to a desired site in a subject can be accomplished by administering one or more cells genetically engineered to express the immune modulating peptide. The genetically engineered cells can comprise a transgene that encodes for the immune modulating peptide.

The genetically engineered cell can comprise a transgene that expresses a MANF family protein. The MANF family protein can be MANF or an active fragment thereof. The transgene can comprise a nucleotide sequence that encodes for MANF or an active fragment thereof, wherein the nucleotide sequence has at least about 70% identity to SEQ ID NO: 28. The transgene can comprise a nucleotide sequence that encodes for MANF or an active fragment thereof, wherein the nucleotide sequence has at least about 80% identity to SEQ ID NO: 28. The transgene can comprise a nucleotide sequence that encodes for MANF or an active fragment thereof, wherein the nucleotide sequence has at least about 90% identity to SEQ ID NO: 28. The transgene can comprise a nucleotide sequence that encodes for MANF or an active fragment thereof, wherein the nucleotide sequence has at least about 95% identity to SEQ ID NO: 28. The transgene can comprise a nucleotide sequence that encodes for MANF or an active fragment thereof, wherein the nucleotide sequence is SEQ ID NO: 28.

The genetically engineered cell can comprise a transgene that expresses a MANF family protein. The MANF family protein can be MANF or an active fragment thereof. The transgene can comprise a nucleotide sequence that encodes for MANF or an active fragment thereof, wherein the nucleotide sequence has at least about 70% identity to SEQ ID NO: 29. The transgene can comprise a nucleotide sequence that encodes for MANF or an active fragment thereof, wherein the nucleotide sequence has at least about 80% identity to SEQ ID NO: 29. The transgene can comprise a nucleotide sequence that encodes for MANF or an active fragment thereof, wherein the nucleotide sequence has at least about 90% identity to SEQ ID NO: 29. The transgene can comprise a nucleotide sequence that encodes for MANF or an active fragment thereof, wherein the nucleotide sequence has at least about 95% identity to SEQ ID NO: 29. The transgene can comprise a nucleotide sequence that encodes for MANF or an active fragment thereof, wherein the nucleotide sequence is SEQ ID NO: 29.

The genetically engineered cell can comprise a transgene that expresses a MANF family protein. The MANF family protein can be a pro-MANF or an active fragment thereof. In one example, the transgene can comprise a nucleotide sequence that encodes for a peptide sequence that has at least about 80% identity to SEQ ID NO:1. In another example, the transgene can comprise a nucleotide sequence that encodes for a peptide sequence that has at least about 90% identity to SEQ ID NO:1. In another example, the transgene can comprise a nucleotide sequence that encodes for a peptide sequence that has at least about 95% identity to SEQ ID NO:1. In another example, the transgene can comprise a nucleotide sequence that encodes for a peptide sequence that has 100% identity with SEQ ID NO:1. In any of these examples, the peptide sequence can have a length that is at least about 5% the length of SEQ ID NO:1. In any of these examples, the peptide sequence can have a length that is at least about 50% the length of SEQ ID NO:1. In any of these examples, the peptide sequence can have a length that is at least about 80% the length of SEQ ID NO:1. In any of these examples, the peptide sequence can have a length that is at least about 90% the length of SEQ ID NO:1. In any of these examples, the peptide sequence can have a length that is at least about 95% the length of SEQ ID NO:1. The peptide sequence, in any of these examples, can also have a maximum length. The maximum length can be, e.g., 100%, 90%, 80%, 70%, 60%, 50%, or 25% the length of SEQ ID NO:1.

The genetically engineered cell can comprise a transgene that expresses a MANF family protein. The MANF family protein can be a pro-MANF or an active fragment thereof. In one example, the transgene can comprise a nucleotide sequence that encodes for a peptide sequence that has at least about 80% identity to SEQ ID NO:2. In another example, the transgene can comprise a nucleotide sequence that encodes for a peptide sequence that has at least about 90% identity to SEQ ID NO:2. In another example, the transgene can comprise a nucleotide sequence that encodes for a peptide sequence that has at least about 95% identity to SEQ ID NO:2. In another example, the transgene can comprise a nucleotide sequence that encodes for a peptide sequence that has 100% identity with SEQ ID NO:2. In any of these examples, the peptide sequence can have a length that is at least about 5% the length of SEQ ID NO:2. In any of these examples, the peptide sequence can have a length that is at least about 50% the length of SEQ ID NO:2. In any of these examples, the peptide sequence can have a length that is at least about 80% the length of SEQ ID NO:2. In any of these examples, the peptide sequence can have a length that is at least about 90% the length of SEQ ID NO:2. In any of these examples, the peptide sequence can have a length that is at least about 95% the length of SEQ ID NO:2. The peptide sequence, in any of these examples, can also have a maximum length. The maximum length can be, e.g., 100%, 90%, 80%, 70%, 60%, 50%, or 25% the length of SEQ ID NO:2.

The genetically engineered cell can comprise a transgene that expresses a MANF family protein. The MANF family protein can be a mature or secreted form of MANF, or an active fragment thereof. In one example, the transgene can comprise a nucleotide sequence that encodes for a peptide sequence that has at least about 80% identity to SEQ ID NO:3. In another example, the transgene can comprise a nucleotide sequence that encodes for a peptide sequence that has at least about 90% identity to SEQ ID NO:3. In another example, the transgene can comprise a nucleotide sequence that encodes for a peptide sequence that has at least about 95% identity to SEQ ID NO:3. In another example, the transgene can comprise a nucleotide sequence that encodes for a peptide sequence that has 100% identity with SEQ ID NO:3. In any of these examples, the peptide sequence can have a length that is at least about 5% the length of SEQ ID NO:3. In any of these examples, the peptide sequence can have a length that is at least about 50% the length of SEQ ID NO:3. In any of these examples, the peptide sequence can have a length that is at least about 80% the length of SEQ ID NO:3. In any of these examples, the peptide sequence can have a length that is at least about 90% the length of SEQ ID NO:3. In any of these examples, the peptide sequence can have a length that is at least about 95% the length of SEQ ID NO:3. The peptide sequence, in any of these examples, can also have a maximum length. The maximum length can be, e.g., 100%, 90%, 80%, 70%, 60%, 50%, or 25% the length of SEQ ID NO:3.

The genetically engineered cell can comprise a transgene that expresses a MANF family protein. The MANF family protein can be a synthetic form of MANF, or an active fragment thereof. In one example, the transgene can comprise a nucleotide sequence that encodes for a peptide sequence that has at least about 80% identity to SEQ ID NO:4. In another example, the transgene can comprise a nucleotide sequence that encodes for a peptide sequence that has at least about 90% identity to SEQ ID NO:4. In another example, the transgene can comprise a nucleotide sequence that encodes for a peptide sequence that has at least about 95% identity to SEQ ID NO:4. In another example, the transgene can comprise a nucleotide sequence that encodes for a peptide sequence that has 100% identity with SEQ ID NO:4. In any of these examples, the peptide sequence can have a length that is at least about 5% the length of SEQ ID NO:4. In any of these examples, the peptide sequence can have a length that is at least about 50% the length of SEQ ID NO:4. In any of these examples, the peptide sequence can have a length that is at least about 80% the length of SEQ ID NO:4. In any of these examples, the peptide sequence can have a length that is at least about 90% the length of SEQ ID NO:4. In any of these examples, the peptide sequence can have a length that is at least about 95% the length of SEQ ID NO:4. The peptide sequence, in any of these examples, can also have a maximum length. The maximum length can be, e.g., 100%, 90%, 80%, 70%, 60%, 50%, or 25% the length of SEQ ID NO:4.

The genetically engineered cell can comprise a transgene that expresses a MANF family protein. The MANF family protein can be CDNF, or an active fragment thereof. The transgene can comprise a nucleotide sequence that encodes for CDNF or an active fragment thereof, wherein the nucleotide sequence has at least about 70% identity to SEQ ID NO:30. The transgene can comprise a nucleotide sequence that encodes for CDNF or an active fragment thereof, wherein the nucleotide sequence has at least about 80% identity to SEQ ID NO:30. The transgene can comprise a nucleotide sequence that encodes for CDNF or an active fragment thereof, wherein the nucleotide sequence has at least about 90% identity to SEQ ID NO:30. The transgene can comprise a nucleotide sequence that encodes for CDNF or an active fragment thereof, wherein the nucleotide sequence has at least about 95% identity to SEQ ID NO:30. The transgene can comprise a nucleotide sequence that encodes for CDNF or an active fragment thereof, wherein the nucleotide sequence is SEQ ID NO:30.

TABLE 5
Human MANF cDNA Sequences.

| SEQ ID | | | |
|---|---|---|---|
| Number | NAME | ASCESSION | SEQUENCE |
| SEQ ID | Human | NM_006010 | agctacggcg cgcggccggg acttggaggc |
| NO: 28 | Pro-MANF | | ggtgcggcgc ggcgggtgcg gttcagtcgg |
| | cDNA | | tcggcggcgg cagcggagga ggaggaggag |
| | | | gaggaggatg aggaggatga ggaggatgtg |
| | | | ggccacgcag gggctggcgg tggcgctggc |
| | | | tctgagcgtg ctgccgggca gccgggcgct |
| | | | gcggccgggc gactgcgaag tttgtatttc |
| | | | ttatctggga agattttacc aggacctcaa |
| | | | agacagagat gtcacattct caccagccac |
| | | | tattgaaaac gaacttataa agttctgccg |
| | | | ggaagcaaga ggcaaagaga atcggttgtg |
| | | | ctactatatc ggggccacag atgatgcagc |
| | | | caccaaaatc atcaatgagg tatcaaagcc |
| | | | tctggcccac cacatccctg tggagaagat |
| | | | ctgtgagaag cttaagaaga aggacagcca |
| | | | gatatgtgag cttaagtatg acaagcagat |
| | | | cgacctgagc acagtggacc tgaagaagct |
| | | | ccgagttaaa gagctgaaga agattctgga |
| | | | tgactggggg gagacatgca aaggctgtgc |
| | | | agaaaagtct gactacatcc ggaagataaa |
| | | | tgaactgatg cctaaatatg cccccaaggc |
| | | | agccagtgca cggaccgatt tgtagtctgc |
| | | | tcaatctctg ttgcacctga gggggaaaaa |
| | | | acagttcaac tgcttactcc caaaacagcc |
| | | | tttttgtaat ttatttttta agtgggctcc |
| | | | tgacaatact gtatcagatg tgaagcctgg |
| | | | agctttcctg atgatgctgg ccctacagta |
| | | | cccccatgag gggattccct tccttctgtt |
| | | | gctggtgtac tctaggactt caaagtgtgt |
| | | | ctgggatttt tttattaaag aaaaaaaatt |
| | | | tctagctgtc cttgcagaat tatagtgaat |
| | | | accaaaatgg ggttttgccc caggaggctc |
| | | | ctaaaaaaaa aaaaaaaaaa aaaaaaaaaa |
| | | | aaa |
| SEQ ID | Human | | atgtgggcca cgcaggggct ggcggtggcg |
| NO: 29 | Pro-MANF | | ctggctctga gcgtgctgcc gggcagccgg |
| | cDNA | | gcgctgcggc cgggcgactg cgaagtttgt |
| | (Coding | | atttcttatc tgggaagatt ttaccaggac |
| | Region | | ctcaaagaca gagatgtcac attctcacca |
| | Only) | | gccactattg aaaacgaact tataaagttc |
| | | | tgccgggaag caagaggcaa agagaatcgg |
| | | | ttgtgctact atatcggggc cacagatgat |
| | | | gcagccacca aaatcatcaa tgaggtatca |
| | | | aagcctctgg cccaccacat ccctgtggag |
| | | | aagatctgtg agaagcttaa gaagaaggac |
| | | | agccagatat gtgagcttaa gtatgacaag |
| | | | cagatcgacc tgagcacagt ggacctgaag |
| | | | aagctccgag ttaaagagct gaagaagatt |
| | | | ctggatgact ggggggagac atgcaaaggc |
| | | | tgtgcagaaa agtctgacta catccggaag |
| | | | ataaatgaac tgatgcctaa atatgccccc |
| | | | aaggcagcca gtgcacggac cgatttgtag |

The genetically engineered cell can comprise a transgene that expresses a MANF family protein. The MANF family protein can be CDNF, or an active fragment thereof. The transgene can comprise a nucleotide sequence that encodes for CDNF or an active fragment thereof, wherein the nucleotide sequence has at least about 70% identity to SEQ ID NO:31. The transgene can comprise a nucleotide sequence that encodes for CDNF or an active fragment thereof, wherein the nucleotide sequence has at least about 80% identity to SEQ ID NO:31. The transgene can comprise a nucleotide sequence that encodes for CDNF or an active fragment thereof, wherein the nucleotide sequence has at least about 90% identity to SEQ ID NO:31. The transgene can comprise a nucleotide sequence that encodes for CDNF or an active fragment thereof, wherein the nucleotide sequence has at least about 95% identity to SEQ ID NO:31. The transgene can comprise a nucleotide sequence that encodes for CDNF or an active fragment thereof, wherein the nucleotide sequence is SEQ ID NO: 31.

The genetically engineered cell can comprise a transgene that expresses a MANF family protein. The MANF family protein can be a pro-CDNF or an active fragment thereof. In one example, the transgene can comprise a nucleotide sequence that encodes for a peptide sequence that has at least about 80% identity to SEQ ID NO:5. In another example, the transgene can comprise a nucleotide sequence that encodes for a peptide sequence that has at least about 90% identity to SEQ ID NO:5. In another example, the transgene can comprise a nucleotide sequence that encodes for a peptide sequence that has at least about 95% identity to SEQ ID NO:5. In another example, the transgene can comprise a nucleotide sequence that encodes for a peptide sequence that has 100% identity with SEQ ID NO:5. In any of these examples, the peptide sequence can have a length that is at least about 5% the length of SEQ ID NO:5. In any of these examples, the peptide sequence can have a length that is at least about 50% the length of SEQ ID NO:5. In any of these examples, the peptide sequence can have a length that is at least about 80% the length of SEQ ID NO:5. In any of these examples, the peptide sequence can have a length that is at least about 90% the length of SEQ ID NO:5. In any of these examples, the peptide sequence can have a length that is at least about 95% the length of SEQ ID NO:5. The peptide sequence, in any of these examples, can also have a maximum length. The maximum length can be, e.g., 100%, 90%, 80%, 70%, 60%, 50%, or 25% the length of SEQ ID NO:5.

The genetically engineered cell can comprise a transgene that expresses a MANF family protein. The MANF family protein can be a mature or secreted form of CDNF, or an active fragment thereof. In one example, the transgene can comprise a nucleotide sequence that encodes for a peptide sequence that has at least about 80% identity to SEQ ID NO:6. In another example, the transgene can comprise a nucleotide sequence that encodes for a peptide sequence that has at least about 90% identity to SEQ ID NO:6. In another example, the transgene can comprise a nucleotide sequence that encodes for a peptide sequence that has at least about 95% identity to SEQ ID NO:6. In another example, the transgene can comprise a nucleotide sequence that encodes for a peptide sequence that has 100% identity with SEQ ID NO:6. In any of these examples, the peptide sequence can have a length that is at least about 5% the length of SEQ ID NO:6. In any of these examples, the peptide sequence can have a length that is at least about 50% the length of SEQ ID NO:6. In any of these examples, the peptide sequence can have a length that is at least about 80% the length of SEQ ID NO:6. In any of these examples, the peptide sequence can have a length that is at least about 90% the length of SEQ ID NO:6. In any of these examples, the peptide sequence can have a length that is at least about 95% the length of SEQ ID NO:6. The peptide sequence, in any of these examples, can also have a maximum length. The maximum length can be, e.g., 100%, 90%, 80%, 70%, 60%, 50%, or 25% the length of SEQ ID NO:6.

The genetically engineered cell can comprise a transgene that expresses a MANF family protein. The MANF family protein can be a synthetic form of CDNF, or an active fragment thereof. In one example, the transgene can comprise a nucleotide sequence that encodes for a peptide sequence that has at least about 80% identity to SEQ ID NO:7. In another example, the transgene can comprise a nucleotide sequence that encodes for a peptide sequence that has at least about 90% identity to SEQ ID NO:7. In another example, the transgene can comprise a nucleotide sequence that encodes for a peptide sequence that has at least about 95% identity to SEQ ID NO:7. In another example, the transgene can comprise a nucleotide sequence that encodes for a peptide sequence that has 100% identity with SEQ ID NO:7. In any of these examples, the peptide sequence can have a length that is at least about 5% the length of SEQ ID NO:7. In any of these examples, the peptide sequence can have a length that is at least about 50% the length of SEQ ID NO:7. In any of these examples, the peptide sequence can have a length that is at least about 80% the length of SEQ ID NO:7. In any of these examples, the peptide sequence can have a length that is at least about 90% the length of SEQ ID NO:7. In any of these examples, the peptide sequence can have a length that is at least about 95% the length of SEQ ID NO:7. The peptide sequence, in any of these examples, can also have a maximum length. The maximum length can be, e.g., 100%, 90%, 80%, 70%, 60%, 50%, or 25% the length of SEQ ID NO:7.

TABLE 6
Human CDNF cDNA Sequences.

| SEQ | | ASCESSION | |
|---|---|---|---|
| ID | NAME | Number | SEQUENCE |
| SEQ | Human CDNF | NM_001029954 | gcgcgggtgg cggaggcgat tgaagctgct |
| ID | cDNA | | ggcccagcat gtggtgcgcg agcccagttg |
| NO: 30 | | | ctgtggtggc cttttgcgcc gggcttttgg |
| | | | tctctcaccc ggtgctgacg cagggccagg |
| | | | aggccggggg gcggccaggg gccgactgtg |
| | | | aagtatgtaa agaattcttg aaccgattct |
| | | | acaagtcact gatagacaga ggagttaact |
| | | | tttcgctgga cactatagag aaagaattga |
| | | | tcagtttttg cttggacacc aaaggaaaag |
| | | | aaaaccgcct gtgctattat ctaggagcca |
| | | | caaaagacgc agccacaaag atcctaagtg |
| | | | aagtcactcg cccaatgagt gtgcatatgc |
| | | | ctgcaatgaa gatttgtgag aagctgaaga |
| | | | agttggatag ccagatctgt gagctgaaat |
| | | | atgaaaaaac actggacttg gcatcagttg |
| | | | acctgcggaa gatgagagtg gcagagctga |
| | | | agcagatcct gcatagctgg ggggaggagt |
| | | | gcagggcctg tgcagaaaaa actgactatg |
| | | | tgaatctcat tcaagagctg gcccccaagt |
| | | | atgcagcgac acaccccaaa acagagctct |
| | | | gatctccaat gccagcacat ttgtgacttg |
| | | | taattagaga gaaaagtgac tctctaggat |
| | | | atggacatgt tgattaagga taactgggaa |
| | | | tgcatcatat ttggtctcat gctttttgtg |
| | | | ttggtattat tcctcagaat tttgttacgt |
| | | | gggtttatga gtgaaactaa tactactgat |
| | | | aacttacatt tgcagtgtac caaaagctaa |
| | | | aagttccttt ctcataagtt tcttggaatg |
| | | | actatgccag ttttcattgc ctgtctccta |
| | | | aaagtgacct actgacaaat tgatggagta |
| | | | aattgattcc aagaaagaag aaggcattca |
| | | | gagactcctc tctggatgca attttaaaat |
| | | | atattggact aaaacaaaag acacaacagt |
| | | | cagcttatct aatgcacaac ttcaatccca |
| | | | aatacagaat caaaagtttt tttcaagtga |
| | | | attttctgtt ttcactctat attgtagctc |
| | | | tctttggtat cagaaatggt caggcaggag |
| | | | tactcgtttt tcccattgga agaaaaccca |
| | | | cttaaaccta cttaaagaaa atagagagat |
| | | | caagaaaccg tataagtata gccaatgttt |
| | | | ggcaattata tcaacattta cattgttaat |
| | | | tgaaccaagc tcaaggtctt tttcattcct |
| | | | tacagtcaca tatcagacaa gtttttgatg |
| | | | gaagattaaa gcatattaaa aacactcata |
| | | | ggc aacttgg |
| SEQ | Human CDNF | | atgtggtgcg cgagcccagt tgctgtggtg |
| ID | cDNA | | gccttttgcg ccgggctttt ggtctctcac |
| NO: 31 | (Coding | | ccggtgctga cgcagggcca ggaggccggg |
| | region | | gggcggccag gggccgactg tgaagtatgt |
| | only) | | aaagaattct tgaaccgatt ctacaagtca |
| | | | ctgatagaca gaggagttaa cttttcgctg |
| | | | gacactatag agaaagaatt gatcagtttt |
| | | | tgcttggaca ccaaaggaaa agaaaaccgc |
| | | | ctgtgctatt atctaggagc cacaaaagac |
| | | | gcagccacaa agatcctaag tgaagtcact |
| | | | cgcccaatga gtgtgcatat gcctgcaatg |
| | | | aagatttgtg agaagctgaa gaagttggat |
| | | | agccagatct gtgagctgaa atatgaaaaa |
| | | | acactggact tggcatcagt tgacctgcgg |
| | | | aagatgagag tggcagagct gaagcagatc |
| | | | ctgcatagct ggggggagga gtgcagggcc |
| | | | tgtgcagaaa aaactgacta tgtgaatctc |
| | | | attcaagagc tggcccccaa gtatgcagcg |
| | | | acacacccca aaacagagct ctga |

The genetically engineered cell(s) that comprise a transgene encoding an immune modulating peptide can be any suitable cell type. The genetically engineered cells can be stem cells. The genetically engineered cells can be produced from stem cells. The genetically engineered cells can be somatic cells isolated from a donor (e.g., a cadaver or organ donor). The genetically engineered cells can be allogenic cells, autologous cells, or cells from a different species (xenogenic). The genetically engineered cells can be genetically engineered fibroblasts. The genetically engineered cells can be encapsulated cells.

### Exemplary Embodiments

In a first aspect, disclosed herein are methods of cell therapy comprising: (a) administering an effective amount of an immune modulating peptide comprising a MANF family protein, or fragment thereof, to a subject in need thereof; (b) administering an effective amount of replacement cells to the subject.

In the methods of the first aspect, the immune modulating peptide can be administered prior to the replacement cells. Alternatively, the immune modulating peptide can be administered concurrently with the replacement cells.

In the methods of the first aspect, the replacement cells can be contacted with the immune modulating peptide prior to administration.

In a second aspect, disclosed herein are methods of cell therapy comprising: (a) administering an effective amount of genetically engineered cells comprising a transgene that expresses an immune modulating peptide comprising a MANF family protein, or fragment thereof, to a subject in need thereof; and (b) administering an effective amount of replacement cells to the subject.

In the methods of the second aspect, the genetically engineered cells can be administered prior to the replacement cells. Alternatively, the genetically engineered cells can be administered concurrently with the replacement cells.

In the methods of the second aspect, the replacement cells can be contacted with the immune modulating peptide prior to administration.

In the methods of the second aspect, the genetically engineered cells can be genetically engineered fibroblasts.

In the methods of the first and second aspects, the MANF family protein can be mesencephalic astrocyte-derived neurotrophic factor (MANF) or a fragment thereof.

In the methods of the first and second aspects, the peptide sequence of the immune modulating peptide can comprise a sequence that has at least about 80% identity with SEQ ID NO:3. For example, the peptide sequence of the immune modulating peptide can comprise a sequence that has at least about 90% identity with SEQ ID NO:3. In another example, the peptide sequence of the immune modulating peptide can comprise a sequence that has at least about 95% identity with SEQ ID NO:3. The immune modulating peptide can have a length that is at least 80% the length of SEQ ID NO:3. For example, the immune modulating peptide can have a length that is 100% the length of SEQ ID NO:3.

In the methods of the first and second aspects, the peptide sequence of the immune modulating peptide can consist of a sequence that has at least about 80% identity with SEQ ID NO:3. For example, the peptide sequence of the immune modulating peptide can consist of a sequence that has at least about 90% identity with SEQ ID NO:3. In another example, the peptide sequence of the immune modulating peptide can consist of a sequence that has at least about 95% identity with SEQ ID NO:3. In another example, the peptide sequence of the immune modulating peptide can consist of a sequence that has 100% identity with SEQ ID NO:3. The immune modulating peptide can have a length that is at least 80% the length of SEQ ID NO:3. For example, the immune modulating peptide can have a length that is 100% the length of SEQ ID NO:3.

In the methods of the first and second aspects, the peptide sequence of the immune modulating peptide can comprise SEQ ID NO:32 and SEQ ID NO:33. For example, the peptide sequence of the immune modulating peptide can consist of a sequence listed in Table 3. The immune modulating peptide can be cell permeable.

In the methods of the first and second aspects, the MANF family protein can be conserved dopamine neurotrophic factor (CDNF) or a fragment thereof.

In the methods of the first and second aspects, the peptide sequence of the immune modulating peptide can comprise a sequence that has at least about 80% identity with SEQ ID NO:6. For example, the peptide sequence of the immune modulating peptide can comprise a sequence that has at least about 90% identity with SEQ ID NO:6. In another example, the peptide sequence of the immune modulating peptide can comprise a sequence that has at least about 95% identity with SEQ ID NO:6. The immune modulating peptide can have a length that is at least 80% the length of SEQ ID NO:6. The immune modulating peptide can have a length that is 100% the length of SEQ ID NO:6.

In the methods of the first and second aspects, the peptide sequence of the immune modulating peptide can consist of a sequence that has at least about 80% identity with SEQ ID NO:6. For example, the peptide sequence of the immune modulating peptide can consist of a sequence that has at least about 90% identity with SEQ ID NO:6. In another example, the peptide sequence of the immune modulating peptide can consist of a sequence that has at least about 95% identity with SEQ ID NO:6. In another example, the peptide sequence of the immune modulating peptide can consist of a sequence that has 100% identity with SEQ ID NO:6. The immune modulating peptide can have a length that is at least 80% the length of SEQ ID NO:6. The immune modulating peptide can have a length that is 100% the length of SEQ ID NO:6.

In the methods of the first and second aspects, the peptide sequence of the immune modulating peptide can comprise SEQ ID NO:34 and SEQ ID NO:35. For example, the peptide sequence of the immune modulating peptide can consist of a sequence listed in Table 4. The neuroprotective peptide can be cell permeable.

In any of the methods of the first and second aspects, the replacement cells can comprise adult stem cells, amniotic stem cells, cord blood stem cells, induced pluripotent stem cells, or a combination thereof. The replacement cells can have been produced from adult stem cells, amniotic stem cells, cord blood stem cells, induced pluripotent stem cells, or a combination thereof. The replacement cells can comprise induced pluripotent stem cells. The replacement cells can have been produced from induced pluripotent stem cells. The replacement cells comprise autologous cells. The replacement cells comprise allogenic cells.

In any of the methods of the first and second aspects, the replacement cells can have been produced using a method that does not destroy an embryo capable of developing into a viable organism.

In any of the methods of the first and second aspects, treatment of neurodegenerative diseases can be excluded from the scope of the claims.

In any of the methods of the first and second aspects, the cell therapy method can be for treatment of a brain or spinal cord injury, a heart disease, a liver disease, baldness, missing teeth, a hearing impairment, a retinal disease, a muscular dystrophy, diabetes, an unhealed wound, or a combination thereof.

In some methods of the first and second aspects, the subject can have a retinal disorder. The retinal disorder can be macular degeneration, diabetic eye disease, age-related macular degeneration, branch retinal vein occlusion, central retinal vein occlusion, central retinal artery occlusion, central serous retinopathy, diabetic retinopathy, Fuchs' dystrophy, giant cell arteritis, glaucoma, hypertensive retinopathy, thyroid eye disease, iridocorneal endothelial syndrome, ischemic optic neuropathy, juvenile macular degeneration, macular edema, macular telangioctasia, marfan syndrome, optic neuritis, photokeratitis, retinitis pigmentosa, retinopathy of prematurity, stargardt disease, usher syndrome, Wolfram syndrome, or Leber Congenital Amaurosis. The replacement cells in these methods can comprise retinal pigment epithelium cells. The replacement cells can comprise retinal photoreceptor cells. The replacement cells in these methods can be administered by intravitreal administration, intracameral administration, conjunctival administration, intracorneal administration, intraocular administration, ophthalmic administration, retrobulbar administration, subconjunctival administration, or by transplant. The genetically modified cells in these methods can be administered by intravitreal administration, intracameral administration, conjunctival administration, intracorneal administration, intraocular administration, ophthalmic administration, retrobulbar administration, subconjunctival administration, or by transplant. The immune modulating peptide in these methods can be administered by intravitreal administration, intracameral administration, conjunctival administration, intracorneal administration, intraocular administration, ophthalmic administration, retrobulbar administration, or subconjunctival administration.

In some methods of the first and second aspects, the subject can have a hearing impairment. The hearing impairment can be caused by an ototoxic chemical, radiation, noise, age, or a combination thereof. The replacement cells in these methods can comprise hair cells. The replacement cells, the genetically engineered cells, or the immune modulating peptides in these methods can be administered by intratympanic administration, intracochlear administration, transtympanic injection, or a combination thereof.

In some methods of the first and second aspects, the subject can have a neurodegenerative disease. The neurodegenerative disease can be Parkinson's disease, amyotrophic lateral sclerosis, Alzheimer's disease, Lewy body disease, Huntington's disease, epilepsy, a memory disorder, an adult demyelinating disorder, a childhood myelin disease or glial disorder, or a combination thereof. The replacement cells in these methods can comprise astrocyte-oligodendrocyte glial progenitor cells, neuronal progenitor cells, dopaminergic neurons, medium spiny neurons, GABAergic neurons, cholinergic neurons, or a combination thereof. The replacement cells, the genetically engineered cells, or the immune modulating peptides in these methods can be administered by injection or infusion into a brain region.

In some methods of the first and second aspects, the subject can have a brain or spinal cord injury. The replacement cells in these methods can comprise glial progenitor cells, neural progenitor cells, bone marrow stem cells, or a combination thereof. The subject can have the brain injury and the replacement cells, the genetically engineered cells, or the immune modulating peptides can be administered by injection or infusion into a damaged brain region. The subject can have the spinal cord injury and the replacement cells, the genetically engineered cells, or the immune modulating peptides can be administered by injection or infusion into the spinal cord or the spinal canal.

In some methods of the first and second aspects, the subject can have a heart disease. The replacement cells in these methods can comprise skeletal myoblasts, bone marrow-derived cells, cardiac stem cells, mesenchymal stem cells, contractile cardiomyocytes, cardiac progenitors, endothelial cells, smooth muscle cells, or a combination thereof. The replacement cells, the genetically engineered cells, or the immune modulating peptides in these methods can be administered by intravascular infusion, intramyocardial injection, or by scaffold or patch-based epicardial delivery to the myocardium.

In some methods of the first and second aspects, the subject can have a liver disease. The replacement cells in these methods can comprise hepatocytes. The replacement cells, the genetically engineered cells, or the immune modulating peptides in these methods can be administered by infusion or injection through the portal vein into the liver, by direct injection into an extrahepatic site, or by transplantation of the replacement cells into a de-cellularized liver scaffold.

In some methods of the first and second aspects, the subject can have a hearing impairment. The hearing impairment can be caused by an ototoxic chemical, radiation, noise, age, or a combination thereof. The replacement cells in these methods can comprise hair cells. The replacement cells, the genetically engineered cells, or the immune modulating peptides in these methods can be administered by intratympanic administration, intracochlear administration, transtympanic injection, or a combination thereof.

In some methods of the first and second aspects, the subject can have a muscular dystrophy. The replacement cells in these methods can comprise satellite cells, myoblasts, bone marrow cells, blood vessel cells, skeletal myogenic progenitors, or a combination thereof. The replacement cells, the genetically engineered cells, or the immune modulating peptides in these methods can be administered by intramuscular injection or systemic injection.

In some methods of the first and second aspects, the subject can have diabetes. The replacement cells in these methods can comprise pancreatic islet cells. The replacement cells in these methods can comprise pancreatic beta cells. The replacement cells, the genetically engineered cells, or the immune modulating peptides in these methods can be administered by transcutaneous catheter infusion through the liver into the portal vein or by implantation in the pancreas.

In some methods of the first and second aspects, the subject can have an acute wound or slow healing or chronic wound. The replacement cells, the genetically engineered cells, or the immune modulating peptides in these methods can be administered directly to a wound site.

### EXAMPLES

Introduction

Metazoans have evolved sophisticated tissue repair and regeneration mechanisms that allow restoring and preserving tissue function in the wake of damaging insults. These mechanisms involve carefully orchestrated cell-cell interactions within the damaged tissue as well as between the damaged tissue and innate immune cells, governed by secreted factors that stimulate cell proliferation and migration, promote or limit apoptosis, and fight off microbes. Deregulation of these interactions can be a major cause of inflammatory and degenerative diseases and can be an important driver of the age-related decline in tissue regenerative capabilities.

Studies in Drosophila have significantly advanced the understanding of tissue repair and regeneration in metazoans. This work has highlighted an important role for the interaction between hemocytes (Drosophila blood cells with macrophage-like activities) and damaged epithelia in the repair process. Hemocytes are activated in response to tissue damage and coordinate localized and systemic repair responses, but have also been implicated in inflammatory processes in flies. Hemocytes are important to the repair process because hemoless (depleted in hemocytes) flies show enhanced tissue loss. A productive model for the genetic dissection of tissue/hemocyte interactions in repair processes is the pupal retina, which responds to UV damage by inducing photoreceptor apoptosis in a dose-dependent manner. A paracrine interaction between UV-damaged photoreceptors and hemocytes through the Pvf-1/PvR pathway governs repair of the damaged retina: damaged photoreceptors secrete Pvf-1 and activate PvR in hemocytes, promoting repair of UV-induced tissue damage through unknown mechanisms (FIG. 1A). Similar interactions are likely to be important for retinal repair in vertebrates, where recruitment of monocyte-derived macrophages (mammalian equivalents of hemocytes) with anti-inflammatory profiles may play an important role in limiting retinal degeneration.

Microglia, monocyte-derived macrophages, and other innate immune cell types can both promote and resolve inflammation, and recent studies have highlighted the importance of managing these inflammatory responses for tissue repair and regeneration. In the central nervous system (CNS), resident (microglia) and invading innate immune cells orchestrate a complex response to damage aimed at restoring tissue integrity. While inflammation can cause neurodegeneration in the brain and retina, innate immune cells can also aid in the retinal repair process, secreting neuroprotective factors that act on photoreceptors and retinal ganglion cells, inhibiting or resolving neurotoxic inflammation. In particular, recruitment of ‘healing’ monocyte-derived macrophages can be important for retinal ganglion cell neuroprotection and oncomodulin-expressing innate immune cells can play a role in axonal regeneration.

This antagonism can be, at least in part, a consequence of different states of immune cell activation. Classical or M1 activation can be associated with pro-inflammatory conditions that can cause tissue damage, while alternative or M2 activation can be associated with resolution of inflammation and tissue repair. Because of these opposing effects of different immune cell phenotypes, immunomodulation rather than immune suppression may be an effective way to promote tissue repair in the retina. Thus, factors that can shift the immune microenvironment towards alternative activation are likely candidates for therapeutic agents in degenerative diseases of the CNS.

Immunomodulation may be of special interest in regenerative approaches relying on stem cell-derived retinal cells. Such regenerative therapies hold promise for the treatment of a range of age-related degenerative diseases. A critical limitation, however, is the fact that aged and diseased tissues provide a poor microenvironment for integration. A case in point are attempts to regenerate the vertebrate retina, a tissue that lacks endogenous stem cells and is subject to a variety of irreversible age-related degenerative pathologies. Human pluripotent stem cells can provide a virtually unlimited source of photoreceptors and retinal pigment epithelial (RPE) cells for replacement and restoration of vision, yet the poor integration efficiency of transplanted cells into the host retina has limited clinical applications. Retinal diseases targeted by this therapeutic approach, such as age-related macular degeneration (AMD) or Retinitis Pigmentosa (RP), are characterized by microglial activation and pro-inflammatory microenvironments that can negatively affect integration and repair. Thus, a better understanding of basic tissue repair mechanisms, and of the impact of inflammation on tissue repair, may lead to the development of rational therapeutic approaches toward improved integration efficiency.

The examples test this hypothesis. Using the Drosophila model for retinal tissue repair, Mesencephalic Astrocyte-derived Neurotrophic Factor (MANF) is identified as an evolutionarily conserved molecule with cytoprotective activity that can be used as a modulator of the tissue immune-microenvironment and to improve the efficiency of regenerative therapies. In flies, MANF is secreted by hemocytes in response to PvR signaling, and stimulates, in an autocrine loop, the transition of hemocytes to a morphology and function that promotes retinal repair. In mice, MANF promotes recruitment of anti-inflammatory macrophages to the retina in the mouse eye. Strikingly, this action of MANF is found to be sufficient to allay light-induced retinal damage, delay retinal degeneration in mouse models for Retinitis Pigmentosa and Leber Congenital Amaurosis, and to promote integration efficiency of exogenously supplied retinal photoreceptor cells. These findings identify a new role for MANF as an immune modulator for tissue repair, and pinpoint a new strategy to improve regenerative therapies in aged and damaged tissues.

### Example 1: MANF is Activated Downstream of Pvf-1/PvR Paracrine Signaling to Promote Retinal Repair in Drosophila

This example shows that, in flies, MANF is secreted by hemocytes in response to PvR signaling, and stimulates, in an autocrine loop, the transition of hemocytes to a morphology and function that promotes retinal repair.

Materials and Methods

Drosophila Stocks and Culture

Fly stocks were raised on standard cornmeal and molasses-based food. All experiments were performed at 25° C. Both sexes gave the same result in all experiments, unless otherwise described.

Fly lines GMR:Grim, Hml:Gal4 and W¹¹¹⁸ were provided by the Bloomington Drosophila Stock Center. RNAi lines PvrRNAiv105353 (UAS:PvRi), KdelRRNAiv9235 (UAS:KdelRi(35)) and KdelRRNAiv9236 (UAS:KdelRi(36)) were obtained from the Vienna Drosophila RNAi Center. The following lines were received as gifts: GMR:Gal4; UAS:Pvf1; UAS:MANF, MANF^{mut96} and MANF^{mut112}; GMR:Rh1G69D.

UV Damage in Drosophila Pupae Retina and Larvae

Pupae retinas were exposed 17.5 mJ of UV light. Second instar larvae were exposed to 50 mJ of UV light.

Pupae of the desired genotype, raised at 25° C. as described above were collected 24 hours after puparium formation and transferred to an apple plate. The anterior pupal case was removed to expose the developing retinas and pupae were arranged laterally to expose the right retina while shielding the left eye. UV irradiation was administered at 17.5 mJ by a UV crosslinker (Stratalinker 1800) and treated animals were reared in the dark until eclosion and aged for 5 days before analysis. Dissected heads were imaged using a Zeiss Discovery V20 stereoscope and the adult eye size (in pixels) quantified using ImageJ software after scale normalization (Rasband, W. S., ImageJ, U. S. National Institutes of Health, Bethesda, Md., USA, http://imagej.nih.gov/ij/, 1997-2014.). Relative eye size was determined by the ratio in the size of the irradiated eye (right) and the shielded eye (left) quantified from the same fly in the same image.

UV Damage in Drosophila Larvae

Crosses were set up to allow egg laying on standard apple plates (with yeast paste) for 12 hr. Approximately 60-65 h after egg laying, larvae of the desired genotypes were arranged on chilled apple plates on ice (approx. 30/plate), dorsal side up. Untreated controls were sorted and arranged (dorsal side up) on ice before being put at 25° C. to develop. UV-treated larvae were irradiated using a UV crosslinker (Stratalinker 1800) at 50 mJ and then allowed to develop at 25° C. Hemocyte samples were collected 24 h after UV exposure and analyzed as described below.

IHC of Hemocyte Smears

Five pairs of larvae were washed in water, dried, and bled by tearing the larval epidermis with two pairs of forceps into a 10 mL drop of PBS placed over Parafilm. To maximize the recovery of hemolymph and circulating cells, the larvae were torn inside out, into the drop. The drop was collected into one well of Millicell EZ SLIDE 8-well glass slide (EMD Millipore) containing 200 μL of 4% PFA. Cell suspension was fixed and cytospined by centrifuging at 2000 rpm for 10 min and washed 3 times in PBS at RT, blocked with 10% donkey serum (Jackson ImmunoResearch) in PBS and incubated with primary antibody (see below) diluted in blocking solution for one hour at RT. Primary antibody was washed 3 times with PBS and detected using alexa conjugated secondary antibodies (Invitrogen, Molecular Probes). Secondary antibody was washed 3 times with PBS and smears were counterstained with DAPI and mounted as described above for retinal cryosections.

TABLE 1-1
Primary IHC antibodies

| Target | | | Dilution |
|---|---|---|---|
| Gene | Species | Source | in IHC |
| MANF | rabbit | Sigma (SAB3500384) | 1:300 |
| GFP | mouse | DSHB (GFP-G1, supernatant)* | 1:50 |
| Pdgf-a | rabbit | Santa Cruz (H-77, sc-7958) | 1:50 |
| CD11b | rat | DSHB (M1/70.15.11.5.2, supernatant)* | 1:20 |
| Iba-1 | rabbit | WAKO (019-19741) | 1:250 |
| GFAP | mouse | Sigma (clone GFAP-B4, SAB4100002) | 1:400 |
| Ym1 | Rabbit | Stem Cell Technologies (01404) | 1:50 |
| Arginase1 | Goat | Santa Cruz (V-20, sc-18354) | 1:50 |
| Arginase 1 | mouse | BD Biosciences (610708) | 1:100 |
| Recoverin | | Gift from Dr. James Hurley, Univ. | 1:10000 |
| | | Washington | |
| Gr-1 | rat | AbD Serotec (RB6-8C5) | 1:100 |
| F4/80 | rat | AbD Serotec (ClA3-1) | 1:100 |
| CD68 | rat | AbD Serotec (FA-11) | 1:100 |
| Atilla | mouse | Gift from Dr. I. Ando, e Hungarian | 1:50 |
| | | Academy of Sciences | |

*The GFP-G1 and M1/70.15.11.5.2 monoclonal antibodies, developed by J.R. Sanes/M. Yamagata (Center for Brain Science, Cambridge, MA) and T. Springer (Center for Blood Research, Boston, MA) respectively, were obtained from the Developmental Studies Hybridoma Bank, created by the NICHD of the NIH and maintained at The University of Iowa, Department of Biology, Iowa City, IA 52242.

Histological Analysis, Imaging and Quantification Methods

Retinal sections and hemocyte smears were analyzed by IHC and other histological methods (see Extended Experimental procedures for details), imaged using a LSM 700 confocal laser scanning microscope and images were used for quantification purposes.

Quantification of Hemocyte Smears

Atilla+, Hml:GFP-high+, MANF+ and Arg+ cells were quantified in 1024×1024 px fields from confocal captures of hemocyte smears stained with the corresponding antibodies and DAPI. DAPI was used to determine the total number of hemocytes/field. Quantifications include 4/5 fields from 3-5 independent smears from at least 2 independent experiments.

Reverse-Transcription and Real-Time Quantitative PCR (RT-qPCR)

Total RNA from hemocytes (obtained from bleeding 20 larvae/sample, as described above for hemocyte smears) or from mouse retinas, were extracted using TRIzol and cDNA was synthesized using 250-500 ng of RNA and iScript cDNA synthesis kit (BioRad). Real-time PCR was performed on a Bio-Rad CFF96™ detection system, using SsoAdvanced™ Universal SYBR® Green Supermix (BioRad). Quantification of expression for each gene in each sample was normalized to actin5c (or beta-actin for mouse samples) and results are shown as gene expression levels relative to levels in control samples which are arbitrarily set to one. All quantifications include three to ten individual samples from multiple experiments.

RNA Sequencing

Larval hemolymph was isolated in PBS and centrifuged to concentrate hemocytes. Total RNA was extracted using Trizol reagent and used as template to generate RNASeq libraries for Illumina sequencing (TruSeq Stranded mRNA LT Kit). Libraries were sequenced using an Illumina MiSeq sequencer. Between 6 and 10 Million 300 bp reads were generated and mapped to the Drosophila genome Release 5.74. Expression was recorded as RPKM (Reads Per Kilobase of transcript per Million mapped reads) and further analyzed using Excel.

TABLE 1-2
Primer list for RT-qPCR

| Gene | Species | Forward Primer | Reverse Primer |
|---|---|---|---|
| MANF | mouse | CACCAGCCACTATTGAAGAAGA | AGCATCATCTGTGGCTCCAA |
| | | (SEQ ID NO: 36) | (SEQ ID NO: 37) |
| Beta-actin | mouse | GCTCTGGCTCCTAGCACCAT | GCCACCGATCCACACAGAGT |
| | | (SEQ ID NO: 38) | (SEQ ID NO: 39) |
| MANF | Drosophila | AGAAGGACGCACAAATCTGC | CGCGTACCTTCAGCTTCTTC |
| | | (SEQ ID NO: 40) | (SEQ ID NO: 41) |
| Pv12 | Drosophila | GGCCTATCACTTCGATGGA | GTCTCAATGTGAACCGAATCC |
| | | (SEQ ID NO: 42) | (SEQ ID NO: 43) |
| Hml | Drosophila | CCGATGATGACGACGAGGAT | GTTCGTTCCGTTCGTTCTCC |
| | | (SEQ ID NO: 44) | (SEQ ID NO: 45) |
| Arg | Drosophila | GCCCAACTGCTTGCCCAA | CCTCGTAGGGGTCAATGTCC |
| | | (SEQ ID NO: 46) | (SEQ ID NO: 47) |
| KdelR | Drosophila | TGAATATCTTTCGCTTTGCTGG | AGCAGCAGTATTATGATGGC |
| | | (SEQ ID NO: 48) | (SEQ ID NO: 49) |
| Actin 5c | Drosophila | AAGTTGCTGCTCTGGTTGTCG | GCCACACGCAGCTCATTGTAG |
| | | (SEQ ID NO: 50) | (SEQ ID NO: 51) |

Western Blots

For whole hemolymph samples 30 larvae/sample were bled into 10 μL of ice-cold RIPA buffer supplemented with protease inhibitors. For plasma and cell fraction separation of hemolymph samples, 60 larvae/sample were bled into 10 μL of ice-cold PBS, the cell and plasma fractions were separated by centrifugation and the cellular fraction re-suspended in 10 μL of ice-cold RIPA buffer supplemented with protease inhibitors. Lentiviral infected human fibroblasts were dissociated off the plates and cells lysed with RIPA buffer supplemented with protease inhibitors. Analyses were performed on 15% SDS-PAGE. After electrophoretic transfer of proteins from SDS-PAGE gels to nitrocellulose membranes using a Trans Blot Turbo Transfer system (BioRad), the membranes were blocked with Tris-buffered saline-0.1% Tween 20 containing 5% milk for 1 h and incubated overnight at 4° C. with primary antibodies. Membranes were then washed and incubated 1 h with a peroxidase-conjugated secondary antibody (1:5000; BioRad, 170-65-150), and developed using the Pierce ECL western blotting substrate (ThemoScientific, 32209).

TABLE 1-3
Primary antibodies

| Target | | | Dilution |
|---|---|---|---|
| Gene | Species | Source | in IHC |
| MANF | rabbit | Sigma (SAB3500384) | 1:1000 |
| GFP | rabbit | Clonthec (632593) | 1:1000 |
| Actin | rabbit | Cell Signaling (4967S) | 1:5000 |

Statistical Analysis

All counts are presented in this Example and in the Examples below as average and standard error of mean (s.e.m.). Statistical analysis was carried out using Microsoft Excel and student's t-test was used to determine statistical significance.

Results

Because innate immune cells can influence tissue repair and inflammation in both flies and vertebrates, it was hypothesized that these cells secrete evolutionarily conserved factors that might help promote repair and regeneration in the retina. To identify such factors, an established model of UV-induced tissue damage in Drosophila was used and RNA sequencing (RNAseq) was performed on isolated hemocytes to identify genes encoding secreted proteins that were induced after epithelial damage in larvae. PvR signaling can be required for hemocyte-mediated tissue repair, and therefore genes induced in a PvR-dependent manner were focused on (FIG. 8). As illustrated in FIG. 8A, candidate genes were selected by the following additive criteria: 1) Induction by UV in Hml>GFP larvae greater than 2; 2) Induction by UV in Hml>PvRRNAi larvae smaller than 2; and 3) change in expression levels by PvR loss of function smaller than 2.

Mesencephalic Astrocyte-derived Neurotrophic Factor (MANF) was identified in this screen, and, based on its evolutionarily conserved neurotrophic activity, its potential as a hemocyte-derived retinal repair factor was further explored.

Although MANF has been shown to be expressed in several tissues in flies and mice, its expression in immune cells had not previously been described. MANF is shown to be expressed in fly hemocytes using immunohistochemistry in hemolymph smears from late 2nd instar larvae (FIG. 1B, left). In these smears, hemocytes were identified by GFP expression driven by the hemocyte specific driver Hemolectin::Gal4 (Hml::Ga14). MANF was also detected by western blot in the plasma fraction of the hemolymph, confirming its secretion (FIG. 1B, right). Consistent with the RNAseq data, qRT-PCR analysis revealed that MANF mRNA levels were significantly higher in hemocytes from UV treated larvae compared to untreated controls (FIG. 1C, left), and that this induction was PvR dependent (FIG. 1C, middle). In these experiments, PvR was knocked down by RNAi before UV treatment, in hemocytes specifically, using Hml::Gal4 to express dsRNA#KK101575 (Vienna Drosophila RNAi Center, stock no. 105353). This PvRRNAi construct has no predicted off-targets and efficiently knocks down PvR (FIGS. S1C and S2A). For UV treatments, larvae were exposed to 50mJ of UV at 2nd instar stage and hemocytes collected hours after.

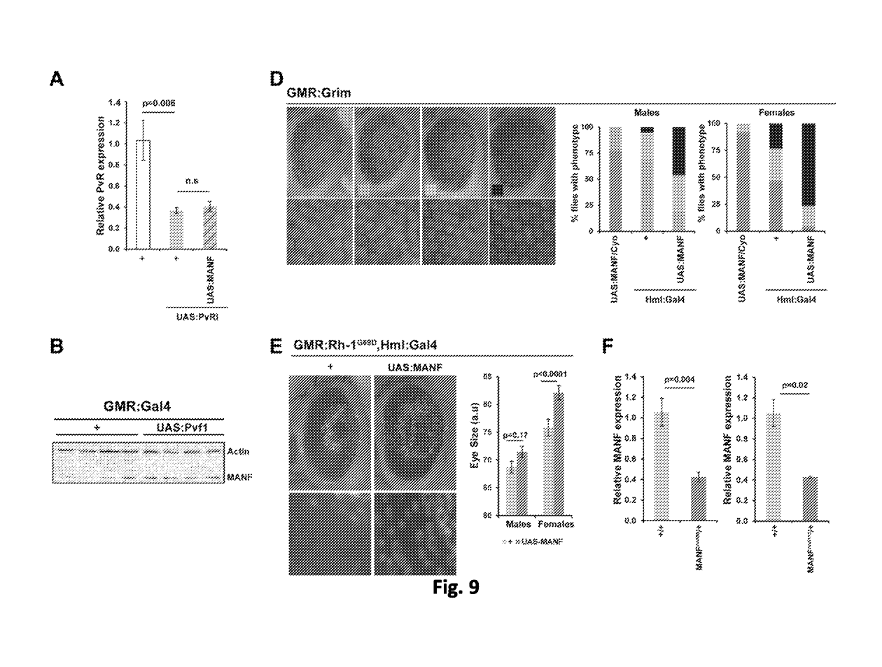

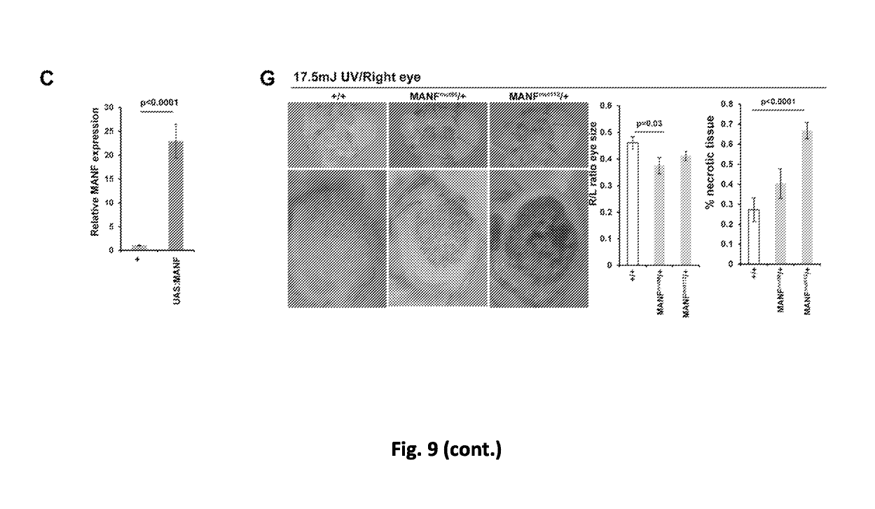

To confirm that Pvf-1/PvR signaling is not only required, but also sufficient to regulate MANF expression in hemocytes, Pvf-1 was overexpressed in the retina (using GMR::Gal4; Glass Multimer Reporter as a driver). This resulted in a significant induction of MANF mRNA specifically in hemocytes, in the absence of damage (FIG. 1C, right) and was accompanied by a significant increase in MANF protein in the hemolymph (FIG. 1D and FIG. 9B). Since Pvf-1 is up-regulated in the retina following tissue damage, and can be required for retinal repair, these data suggested that Pvf-1/PvR-mediated transcriptional induction of MANF in hemocytes, and secretion of MANF into the hemolymph, may be a key factor in the hemocyte-mediated tissue damage response.

This hypothesis was tested by assessing retinal tissue preservation in various models of retinal damage in Drosophila, following overexpression of MANF in hemocytes. Hml::Gal4 was used to over-express MANF in hemocytes before inducing tissue damage by UV-irradiation of the developing pupal retina. In this assay, damage is induced in a post-mitotic tissue; therefore, enhanced tissue preservation is independent of an endogenous regenerative process and is associated with repair mechanisms. Flies overexpressing MANF in hemocytes (FIG. 9C) showed significantly reduced tissue loss after UV exposure, suggesting that hemocyte-derived MANF can promote tissue repair (FIG. 1E-F). Importantly, MANF over-expression in hemocytes was also sufficient to significantly allay the enhanced tissue loss observed in flies in which PvR was knocked down in hemocytes (FIG. 1E-F), without affecting PvRRNAi knock-down efficiency (FIG. 9A). The cytoprotective activity of hemocyte-derived MANF was further confirmed in two other models of retinal damage, in which degeneration is induced by retinal (GMR driven) over-expression of the pro-apoptotic gene grim or of mutant Rhodopsin (Rh1G69D) (FIGS. 9D and E).

To test whether MANF is not only sufficient, but also required for retinal repair, flies carrying null mutations in the manf gene (MANF^{mut96} and MANF^{mut112}) were used. These null alleles are homozygous lethal at early 1st instar larval stages, yet MANF heterozygotes (which express significantly lower levels of MANF in hemocytes compared to wild-types; FIG. 9F) had a significantly increased tissue degeneration response to UV (FIG. 1G-H; FIG. 9G). Importantly, this increase in tissue loss could be rescued by MANF overexpression in hemocytes (FIG. 1G-H).

### Example 2: Damage Response-Associated PDGF-A/MANF Paracrine Signaling is Conserved in Mammals

This Example shows that damaged retinal cells engage an evolutionary conserved retinal damage response that involves the Pvf/PDGF-mediated recruitment/activation of MANF-expressing innate immune cells.

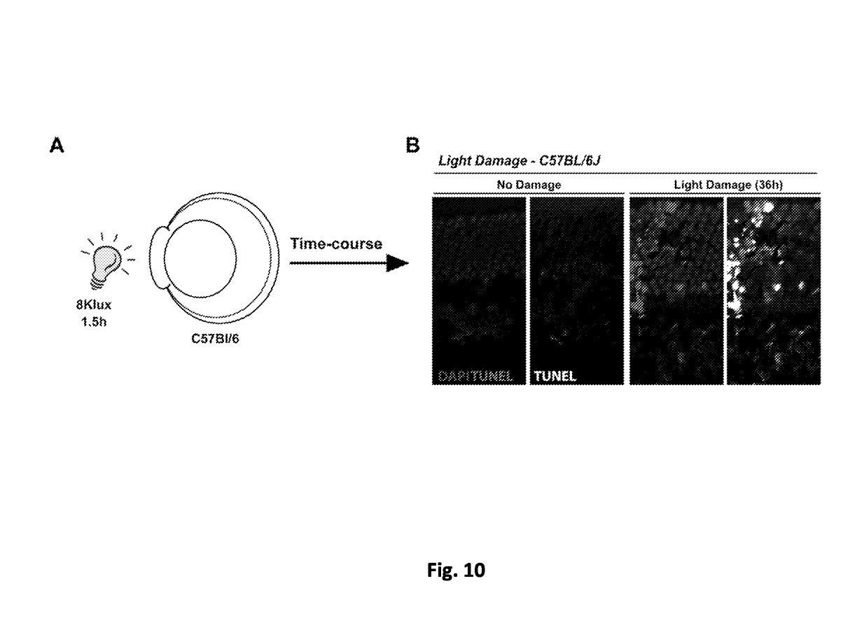

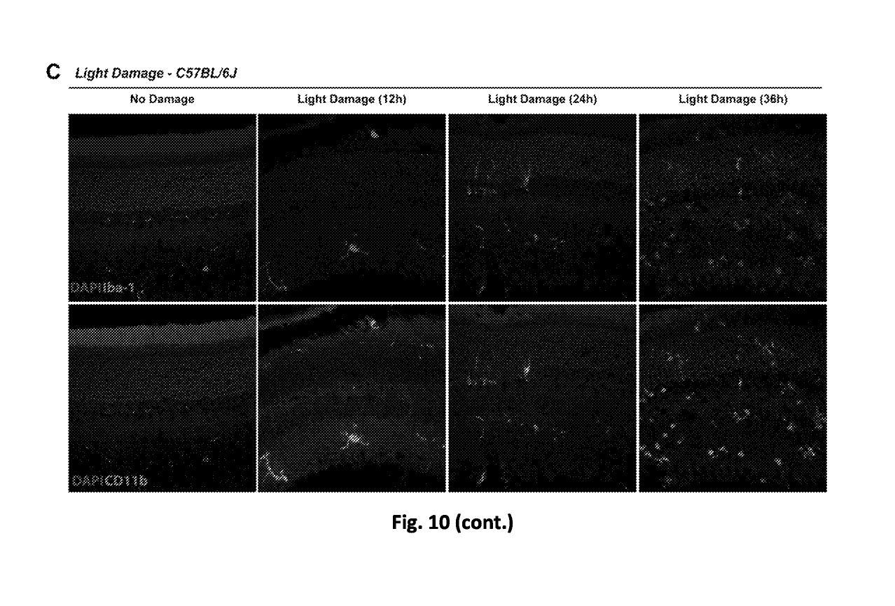

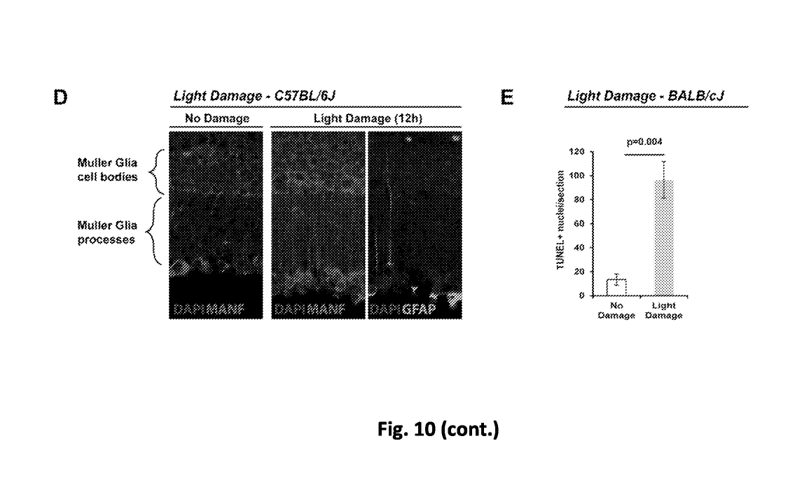

Because MANF is an evolutionarily conserved protein, its potential applications to allay retinal degeneration and improve retinal repair in mammals was explored. In mammals, retinal damage can be accompanied by the activation and recruitment of innate immune cells, including microglia and monocyte-derived macrophages. These localize to the plexiform layers of the retina and the vitreal and choroidal blood vessels, respectively, and invade the damaged tissue in response to injury. This response resembles the recruitment and activation of hemocytes after retinal damage in Drosophila, indicating that the paracrine interaction between damaged retinal cells and innate immune cells may indeed be conserved between flies and vertebrates. This idea was tested using light-induced damage of the mouse retina as a paradigm (FIG. 10A).

Materials and Methods

Mice

All mice used in the described studies were housed and bred at the AAALAC accredited vivarium of The Buck Institute for Research on Aging in a Specific Pathogen Free (SPF) facility and housed in individually ventilated cages on a standard 12:12 light cycle. All procedures were approved by the Buck Institute Institutional Animal Care and Use Committee (IACUC).

BALB/cJ albino (JAX, stock nr. 000651) and C57BL/6J pigmented (JAX, stock nr. 000664) wild type mice were purchased from The Jackson Laboratories (JAX).

Light Damage in Mice

Mice were dark adapted for 18 hours before the procedure. Test eyes were exposed to 5,000 lux (BALB/cJ) or 8,000 lux (C57BL/6J) of bright light using a 144-LED microscope ring light (AmScope) for 1-1.5 hours. After light damage, mice were allowed to recover from anesthesia, returned to their cages and housed in darkness until analysis. Undamaged control mice were housed in regular conditions (see above) throughout the experiment.

Animals were anesthetized for 15 min in an isoflurane chamber and pupils were dilated using a mixture of 5% Phenylephrine (Arcos Organics) and 1% Tropicamide (Alfa Aesar). When intravitreal injections were performed in combination, animals were injected prior to light exposure. Mice were kept under anesthesia during light exposure to ensure direct and constant illumination during the exposure time. Light intensity at the site of exposure was measured using a digital lux meter (LX1330B, Sinometer).

Mouse Retinal Tissue Harvesting and Storage

Whole eyes were dissected out from treated animals at the designated times and fixed for 1 h at 4° C. in 4% Paraformaldehyde in PBS (4% PFA) with a small incision in the cornea to allow intraocular access to fixative. After fixation, the cornea and lens were dissected out and the retinal tissue fixed for an additional hour in fresh 4% PFA. The tissue was washed 3 times with PBS and incubated with 15% sucrose in PBS overnight at 4° C. Sucrose treated tissue was embedded in 7.5% gelatin/15% sucrose in PBS at 37° C. for 2 h and then transferred to cryomolds (Tissuetek) and flash frozen with 2-methylbutane at −80° C. Frozen embedded tissue was cryosectioned at 14 μm thickness, collected in Superfrost Plus microslides (VWR) and stored at −80° C. until analysis.

IHC of Mouse Retinal Cryosections and Nuclei Staining

Cryosections collected in slides were thawed and hydrated in PBS for 15 minutes at room temperature (RT). For nuclei staining, slides were incubated for 5 min in 300 nM DAPI (4′,6-diamidino-2-phenylindole) in PBS at RT, rinsed in PBS and mounted with Fluoromount G media (17984-25, Electron Microscopy Sciences) and micro cover glass No. 2 (48382-128, VWR). For antibody staining, sections were permeabilized with PBS containing 0.1% Tween20 (PBT) for 15 min at RT, blocked with 10% donkey serum (Jackson ImmunoResearch) in PBT and incubated with primary antibody (see below) diluted in blocking solution, overnight at 4° C. Primary antibody was washed 3 times with PBT and detected using alexa conjugated secondary antibodies (Invitrogen, Molecular Probes). Secondary antibody was washed 6 times with PBT and sections were counterstained with DAPI and mounted as described above.

TUNEL Staining of Mouse Retinal Cryosections

Cryosections collected in slides were thawed and hydrated in PBS for 15 minutes at room temperature (RT). Tissue was permeabilized for 2 min in boiling 10 mM Citrate buffer and washed 3 times with PBS after cooling down to RT. Cryosections were incubated in the dark for 1 h at 37° C. with 50 μL of TUNEL reaction mixture (In situ cell death detection kit, TMR Red, Roche) prepared according to manufacturer's instructions. Slides were rinsed three times with PBS, counterstained with DAPI and mounted as described above.

qRT-PCR was performed as described above.

Recombinant Proteins for Intravitreal Injection

mrPDGF-AA (Biovision, Inc, Milpitas, Calif., USA): Mouse Recombinant Platelet-Derived Growth Factor-AA, a disulfide linked dimer comprised of 2 polypeptide chains was produced in E. coli, purchased in lyophilized form and reconstituted in PBS at a concentration of 1 mg/ml. Vehicle for all experiments was PBS (Phosphate Buffered Saline): 10× molecular biology grade PBS (46-013-CM, CORNING cellgro, lot 46013041) was diluted with sterile double distilled water to 1× and the final solution sterilized using a vacuum PVDF 0.22 μm filter system (F-2860-5, BioExpress).

Intravitreal Injection

Animals were anesthetized using isoflurane and kept under anesthesia during the procedure. Recombinant proteins or cells in 1 μL volume were injected into the right eye using a graduated pulled glass pipet and a wire plunger (Wiretrol II, 5-0000-2005, Durmmond Scientific Company).

Procedure:

Following testing the depth of anesthesia, the pipet was used to poke a hole just beyond the corneo-scleral margin. The pipet was further advanced carefully and the solution delivered into the vitreous space. The pipet was kept in place for 30 secs for the intra-ocular pressure to normalize and then gently withdrawn.

After both types of injections, eyes were coated with antibiotic ointment (Neomycin and polymyxin B Sulfates and Bacitracin Zinc ophthalmic Ointment, USP, 1748-235-35, Akorn) and animals were allowed to recover on a warm pad before being transferred to their cages.

Results

Exposure to high levels of bright light induces photoreceptor apoptosis that is dependent on c-fos/AP-1, and independent of photo transduction, recapitulating the response of developing Drosophila photoreceptors to UV irradiation. In model used, photoreceptor apoptosis was moderately induced in C57BL/6J mice exposed to 8 Klux of bright light for 1.5 hours (FIG. 10B), and was associated with microglia activation (FIG. 10C).

To test the conservation of Pvf-1/MANF paracrine signaling in retinal damage responses, a time-course analysis of innate immune cell recruitment and gene expression in the retina following light exposure was performed (FIGS. 2 and 10). PDGF-family and VEGF-family proteins are the mammalian homologs of Pvf-type ligands in Drosophila. PDGF-A expressing cells in the neural retina 6 hours after light damage were detected, while undamaged retinas were devoid of any PDGF-A signal (FIG. 2B). The induction of PDGF-A was followed by a significant increase in MANF transcripts (FIG. 2C) and the detection of MANF+ innate immune cells (FIG. 2D). Invading innate immune cells, identified by CD11b expression, localized to the vitreous, and were first detected twelve hours after light exposure (FIG. 2D, 12h). Resting microglia, localized to the plexiform layers in the absence of damage, did not express MANF (FIG. 2D, no damage). Thirty-six hours later, MANF+ innate immune cells were found within the outer nuclear layer (ONL) (FIG. 2D, 36h). This microglia/innate immune cell activation/recruitment was also accompanied by a re-distribution of MANF protein from the cell bodies of Muller glia (where it is detected in control conditions) to glial processes (identified by staining against Glial fibrillary acidic protein; GFAP, FIG. 10D). Thus, induction of PDGF-type ligands in the damaged retina and the recruitment of MANF+ innate immune cells recapitulate the early events of the retinal/hemocyte interaction observed in the fly.

In order to confirm the paracrine interaction, whether, as seen in flies, PDGF ligands are sufficient to promote MANF expression and innate immune cell recruitment or activation in the absence of damage was tested. Consistent with the existence of a conserved regulatory mechanism, intravitreal injection of mouse recombinant PDGF-AA, in undamaged C57BL/6J mice, resulted in a significant increase of CD11b+ innate immune cells in the eye (FIG. 2E-F). Importantly, CD11b+ cells also expressed MANF and were found within the retina (FIG. 2E, lower inset), in the vitreous (FIGS. 2E and G) and choroidal blood vessels (FIG. 2H).

### Example 3: MANF has a Conserved Neuroprotective Function in the Mammalian Retina

This Example suggests that MANF can generally prevent photoreceptor apoptosis and delay retinal degeneration independently of the damaging stimulus, supporting a model for MANF as an enabler of tissue repair.

Since hemocyte-derived MANF in Drosophila was sufficient to promote retinal repair (FIGS. 1E and F), it was hypothesized that the function of MANF-expressing innate immune cells in the mammalian retina is to promote tissue repair and/or cell survival. It follows that MANF protein supplementation would ameliorate retinal degeneration, and this hypothesis was tested in several mouse models of retinal regeneration.

Materials and Methods

TUNEL was performed as described above.

Light damage in mice was performed as described above.

Mice

Crx^{tvrm65} mice were generated in Dr. Nishina's lab and made available to our lab through The Jackson Laboratories (JAX). Crx^{tvrm65} are C57BL/6J mice carrying the tvrm65 recessive mutation in the last exon of the Crx gene. The mutation is predicted to cause an early termination at Leu277 (TTG) of the 323 aa from the longer isoform or at Leu253 of a 299 aa product from the shorter isoform.

Pde6b^{Rd1} are C3H/HeJ mice purchased from The Jackson Laboratory (JAX, stock nr. 000659). Pde6b^{Rd1} mice carry a spontaneous mutation common among laboratory inbred mice arising from a murine viral insertion in the first intron and a second nonsense mutation in exon 7 of the Pde6b gene.

Quantification of TUNEL nuclei

For experiments with Crx^{tvrm65}, TUNEL+ nuclei were counted directly under the microscope in whole eye sections stained with TUNEL and DAPI. Quantifications include 5 independent sections per eye, in a total of at least 5 eyes collected in two independent experiments.

For experiments with light damage and Pde6b^{Rd1}, TUNEL+ nuclei were counted in 1024×1024 px fields from confocal captures of retinal sections stained with TUNEL and DAPI. Quantifications include at least 3 independent sections per eye, in a total of 3 (Pde6b^{Rd1}) or 8 (Light damage) eyes, collected in 2 (Pde6b^{Rd1}) or 4 (Light damage) independent experiments.

Quantification of Nuclei Rows in the ONL

For all experiments, the number of nuclei in the ONL was determined using 1024×1024 px fields from confocal captures of retinal sections stained with DAPI. Quantifications include 3-5 independent sections per eye, in a total of 3-6 eyes collected in 2 independent experiments.

Quantification of Cells in the Vitreous

For all experiments the total numbers of CD11b+, Ym1+ and Arg1+ cells in the vitreous were counted directly under the microscope in whole eye sections stained with the corresponding antibodies. In all cases, co-staining with DAPI and CD11b was used to identify innate immune cells in the vitreous. Quantifications include 3-5 independent sections per eye, in a total of 5-8 eyes collected in at least two independent experiments.

The percentage of MANF+CD11B+ in light damage retinas were counted in 1024×1024 px fields from confocal captures of retinal sections stained with DAPI and the corresponding antibodies. Quantifications include 5-13 independent sections form a minimum of 4 eyes per condition.

The percentage of Gr1+, F4/80+ and CD68+ cells in Crx^{tvrm65} mice injected with hfib-MANF were counted in 1024×1024 px fields from confocal captures of retinal sections stained with DAPI and the corresponding antibodies. DAPI staining was used to quantify the total number of cells in the vitreous and estimate percentages. Co-staining with MANF and Arg1 was used to quantify fraction of MANF+ and Arg1+ co-expression in the same fields from the same sections. Quantifications include 10 independent sections form a total of 6 eyes from two independent experiments.

Recombinant Proteins for Intravitreal Injection

hrMANF (Icosagen AS, Tartu, Estonia, Batch 030513): Human recombinant mesencephalic astrocyte-derived neurotrophic factor was a kind gift from Amarantus Biosciences Inc. hrMANF was expressed by in a Chinese hamster ovary (CHO)-based cell line using QMCF technology and purified by ion-exchange and gel-filtration chromatography from serum-free CHO growth medium. rhMANF was provided as a 1 mg/ml solution in PBS, pH 7.4

MANF secreting human fibroblasts for intravitreal injection

Plenti6.3 eGFP-hMANF (Gift from M. Henderson and B. Harvey, NIH/NIDA, Baltimore, Md., USA) and EF1α-GFP (Gift from Dr. Charles Murry, UW, Seattle) plasmids were used for MANF and GFP (control fibroblasts) overexpression, respectively. Lentiviral generation was performed as reported. Briefly, the viruses were generated by co-transfection of the vector and the packaging constructs (VSVg, Gag/Pol, Rev) in the 293T cell line by the calcium phosphate precipitation technique. The following day, the precipitates were removed and fresh medium was added. This medium, containing the viral particles, was recovered 48 h later and centrifuged at 3,000×g for 10 min. Subconfluent cultures of human fibroblasts (Coriell Cell Repository, NJ) cultured in 6 well plates were infected by adding to each plate 1 mL of medium containing the viral particles, and 1 μL of polybrene (Sigma). The medium was removed 24 h later, and the cells were then cultured in DMEM containing 20% FBS. Before intravitreal injection, cells from 2 wells were dissociated with trypsin-EDTA (Gibco) and re-suspended in 10 μl of DMEM.

Intravitreal injection was performed as described above.

Results

BALB/cJ albino mice exhibit enhanced light sensitivity due to lack of pigmentation, and exposure to 5 kLux of white light for one hour results in a significant increase in photoreceptor apoptosis (FIG. 10E). In addition, two genetic models of retinal degeneration (Crx^{tvrm65} and Pde6b^{Rd1}) were used. Crx^{tvrm65} mice carry a recessive Crx loss of function mutation leading to photoreceptor degeneration starting at about post-natal day (P) 14 resulting in a reduction of photoreceptor cell bodies to 60% of controls by P21. Homozygotes for the Pde6b^{Rd1} loss of function allele of phosphodiesterase 6b are normal at birth and up to P6, but then exhibit rapid rod photoreceptor degeneration, resulting in the loss of almost all rod photoreceptors by P13. Consequently, a strong reduction in the number of photoreceptor rows is observed in the ONL

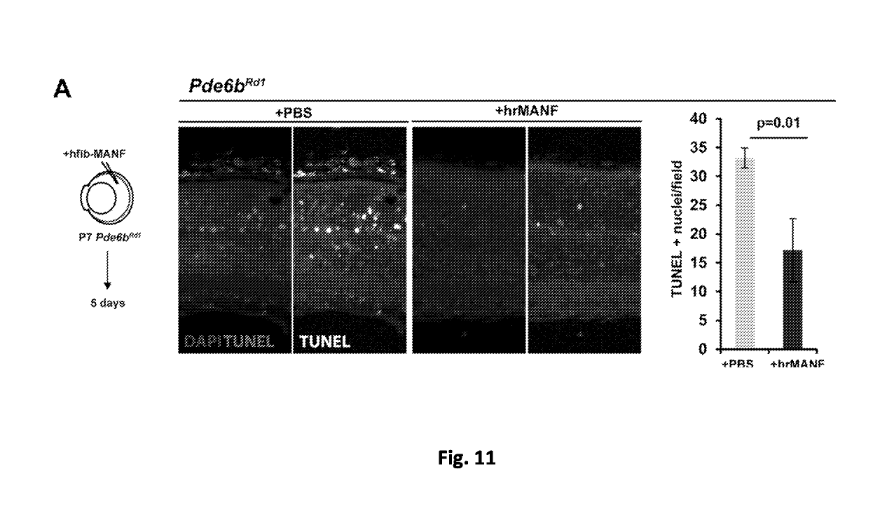

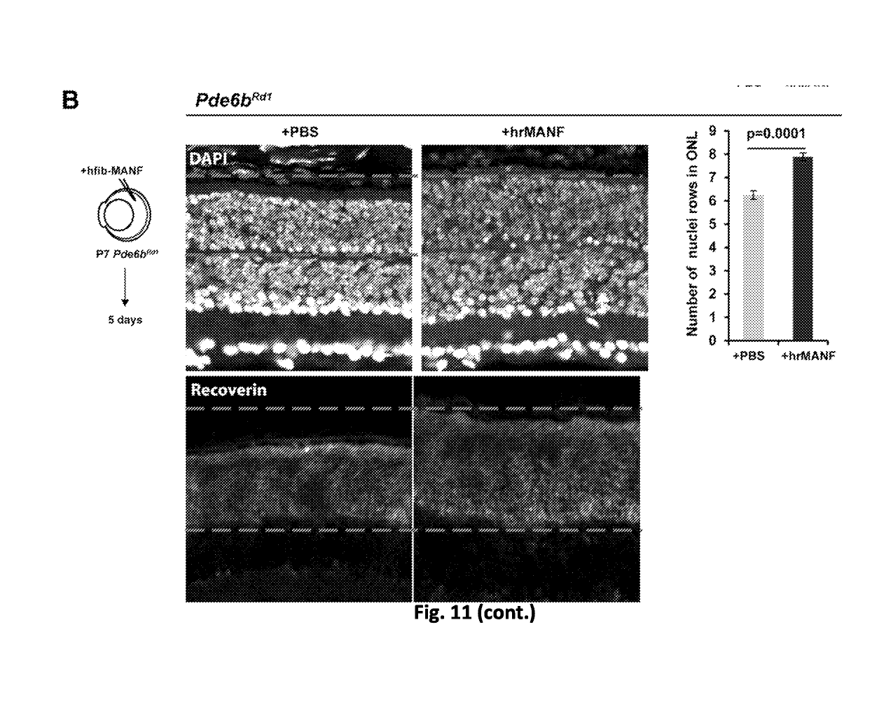

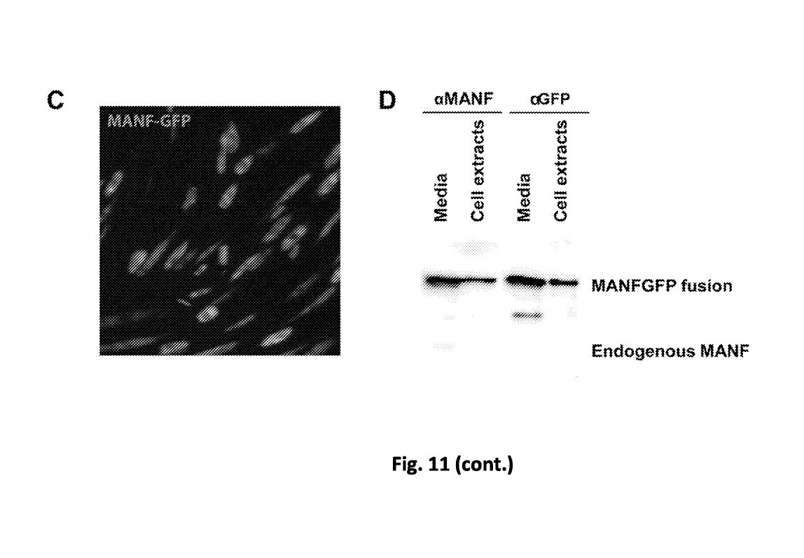

To test if MANF is sufficient to limit photoreceptor degeneration in these models, human recombinant MANF (hrMANF) protein or vehicle (PBS) was injected into the vitreous immediately prior to light exposure or at the onset of retinal degeneration (P14 for Crx^{tvrm65} mice and P7 for Pde6b^{Rd1} mice), and evaluated photoreceptor apoptosis by Terminal deoxynucleotidyl transferase dUTP Nick End Labeling (TUNEL). Indeed, MANF injection was sufficient to significantly reduce apoptosis in all three models of retinal degeneration (FIG. 3A-C and FIG. 11A-B). Light damaged retinas were analyzed two days following light exposure, when apoptotic photoreceptors can be clearly detected. The number of apoptotic nuclei in light damaged eyes injected with PBS was comparable to that of uninjected light-damaged eyes (FIG. 3A and FIG. 10E) and MANF significantly reduced apoptosis, which was now detected only in scattered cells (FIG. 3A). Crx^{tvrm65} mice are a model of slow retinal degeneration and thus at any given time point only scattered apoptotic nuclei are detected in the retina (FIG. 3B, PBS). Yet MANF intravitreal delivery significantly reduced the number of TUNEL+ nuclei two days after injection (FIG. 3B). As photoreceptors degenerate, the number of nuclei in the ONL is reduced, and in Crx^{tvrm65} mice there are on average 5-6 rows left at P21 (FIG. 3C, PBS). In MANF treated eyes, about two additional rows of photoreceptors were preserved one week after delivery (FIG. 3C), suggesting that inhibition of apoptosis effectively slows retinal degeneration in this model Similar results were observed in the Pde6b^{Rd1} mouse model analyzed five days after intravitreal delivery of MANF (FIG. 11A-B).

Finally, whether a persistent source of MANF could further delay retinal degeneration in Crx^{tvrm65} mice was investigated. For this purpose, human fibroblasts were infected with a lentivirus driving the expression of a functional MANF-GFP fusion protein. MANF-GFP expression could readily be detected in these fibroblasts and in the media supernatant, confirming that the fusion protein is efficiently secreted (FIG. 11C-D). When MANF-secreting fibroblasts were injected into the vitreous of P14 Crx^{tvrm65} mice, their retinas degenerated more slowly than control-fibroblast-injected retinas and contained at least one additional row of photoreceptors in the ONL by P28 (FIG. 3D).

### Example 4: MANF is an Immune Modulatory Factor

At least two non-exclusive mechanisms can be envisioned for this role of MANF in tissue repair: 1) MANF acts directly on photoreceptors and promotes survival/inhibits apoptosis, and/or 2) innate immune cell-derived MANF modulates inflammation and creates an immune microenvironment that promotes tissue repair. Inflammatory tissue microenvironments have been associated and causally linked to retinal degeneration, and monocyte-derived macrophages have been implicated in resolving inflammation and promoting tissue repair in several models of CNS injury. Based on these studies, and because recruitment of MANF+ innate immune cells is associated with tissue damage responses in the retina (FIG. 2), a possible immune modulatory function for MANF was investigated. Specifically, whether MANF can enhance tissue repair associated M2 activation of macrophages was tested. Using well-established markers of M2 activation (ym1 and arginase1), MANF-induced changes in the number and activation status of microglia and other innate immune cells in various models of retinal degeneration was assessed.

Materials and Methods

TUNEL and quantification of TUNEL+ cells was performed as described above.

Intavitreal injection with MANF-secreting fibroblasts was performed as described above.

Intavitreal injection with mrPDGF-AA was performed as described above.

Quantification of cells of the vitreous and quantification of nuclei rows in the ONL performed as described above.

Immunohistochemistry performed as described above.

Light damage in mice was performed as described above.

Mice used are described above.

Results

Consistent with a function as an immune modulatory factor, intravitreal injection of MANF-secreting fibroblasts resulted in the detection of MANF+ innate immune cells (CD11b+) in the vitreous of Crx^{tvrm65} mice, which are not detected in the control degenerating retinas (FIG. 3E, top panel). This indicates that in this damage model, enhancement of photoreceptor survival by MANF (FIG. 3B-D) is accompanied by the recruitment of MANF+ innate immune cells. Importantly, these CD11b+ cells with round morphology also expressed markers of M2 activation. The number of M2 (Ym1+) cells was significantly increased in Crx^{tvrm65} retinas supplemented with MANF-secreting fibroblasts (FIG. 3E). Of note, fibroblasts injected into the vitreous were detected by GFP expression at the time of dissection and were completely removed along with the lens, thus do not contributing to the cells counted within the vitreous in these experiments. Intravitreal delivery of hrMANF had similar effects in Crx^{tvrm65} retinas, significantly increasing the number M2 (Arg1+) innate immune cells (FIG. 3F), which are otherwise rare in Crx^{tvrm65} eyes (FIG. 3E-F). These observations were further supported in the light-damage model, in which intravitreal delivery of hrMANF was also sufficient to significantly increase the number of MANF+ and M2 (Arg1+) innate immune cells (FIG. 3G and FIG. 12A-B), found in the vitreous and invading the damaged retina.

Since PDGF-AA was sufficient to recruit MANF+ innate immune cells to the vitreous of non-damaged eyes (FIG. 2E-H) whether supplying PDGF-AA would also be sufficient to enhance M2 populations in the eye was tested. Indeed, recruitment of MANF+ monocyte-derived macrophages in response to PDGF-AA treatment was accompanied by a significant increase in the number of CD11b+ cells co-labeled with M2 markers (Ym1+) (FIG. 3H and FIG. 12C).

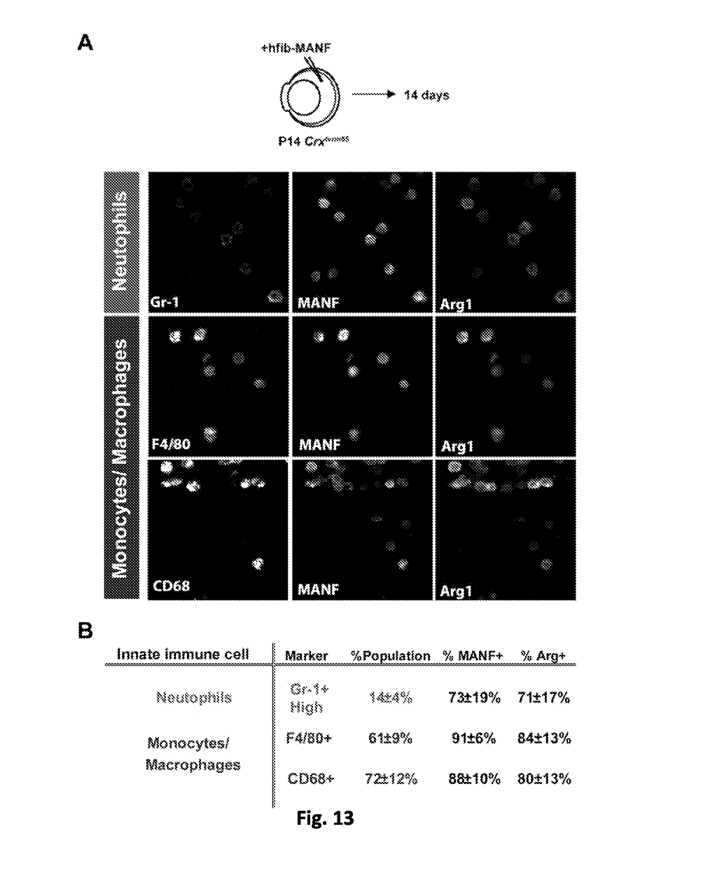

Further analysis of the cell population invading the damaged retinas supplemented with MANF revealed that the innate immune cell population recruited in these conditions was mostly composed by monocytes and monocyte-derived macrophages (60-80%, identified by F4/80 or CD68 expression, FIG. 13). Neutrophils, on the other hand, represented about 15% of the population (FIG. 13). Importantly, the majority of both macrophages and neutrophils expressed MANF and Arg1 (FIG. 13) and about 80% of the MANF expressing cells also expressed Arg1, suggesting that MANF expression is associated with M2 markers.

Together, these data support the notion that MANF modulates immune responses that enhance pro-repair conditions of the immune environment in the retina.

### Example 5: MANF Immune Modulatory Function is Conserved in Drosophila

This Example shows that the immune modulatory function of MANF is conserved in Drosophila and supports the idea that MANF has a conserved immune modulatory function that promotes activation of hemocytes/microglia/macrophages into phenotypes associated with tissue repair.

Materials and Methods

Fly lines used are as described above.

Hemocyte smears performed as described above.

Immunohistochemistry performed as described above.

UV damage to Drosophila performed as described above.

qRT-PCR performed as described above.

Results

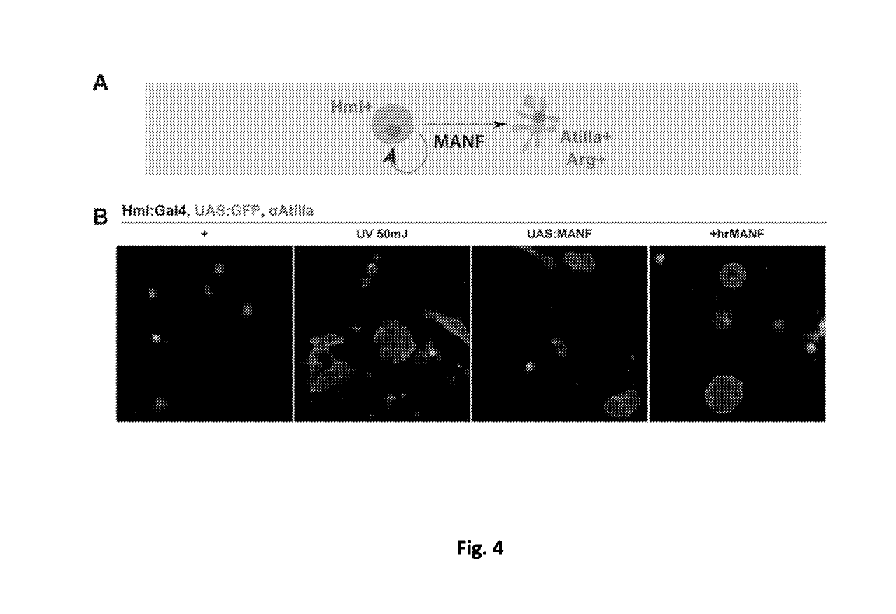

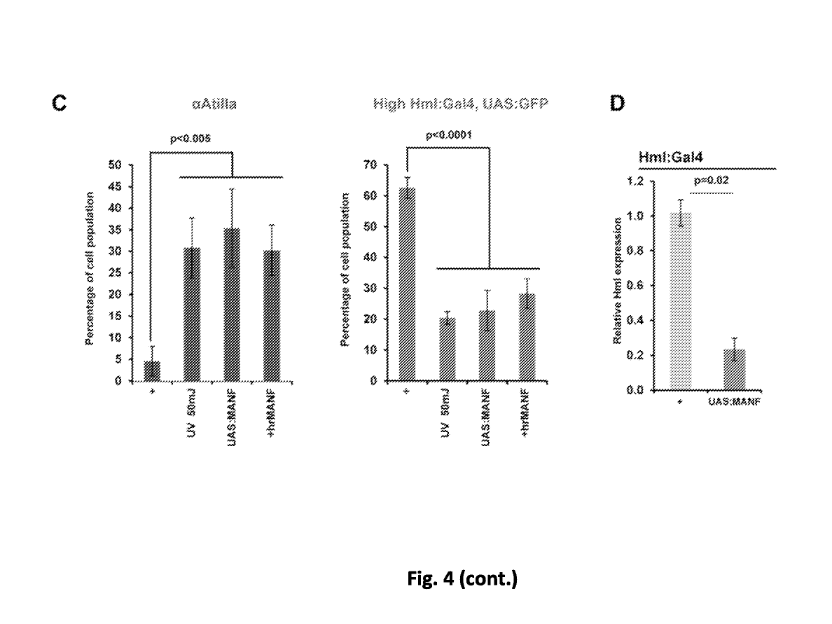

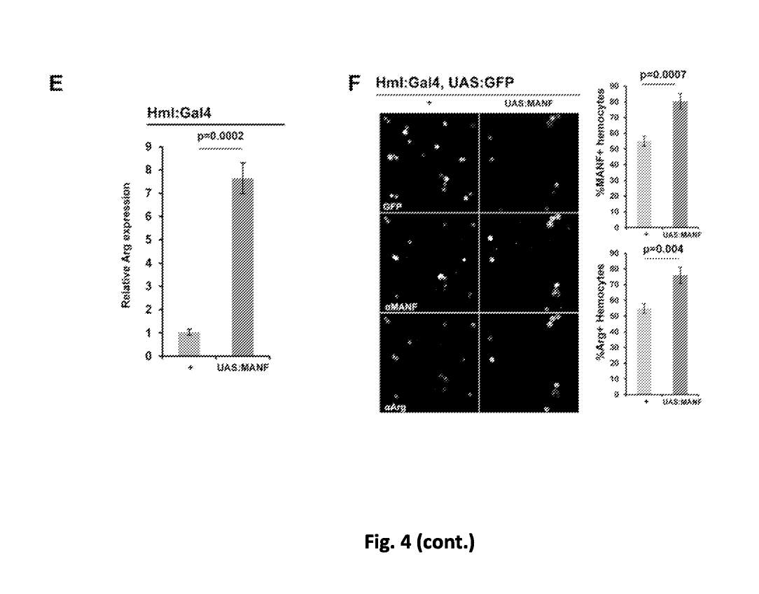

To gain more detailed insight into MANF immune modulatory function and its requirement for tissue repair, the Drosophila system was used. While phenotypes that directly resemble M1/M2 activation in mammals have not been described for fly hemocytes, acquisition of lamellocyte phenotypes, characterized by down-regulation of plasmatocyte makers (hemolectin, hemese), expression of Atilla protein and activation of a specific misshapen enhancer, have been reported in response to sterile wound healing. These cellular phenotypes thus correlate with hemocyte activation and may influence wound healing and tissue repair. In agreement, a significant increase in the proportion of lamellocyte-like cells (detected by atilla expression) and a decrease in the high Hml:GFP population in response to UV-induced epithelial damage in Drosophila larvae was observed (FIG. 4A-C).

To test if MANF has a conserved immune-modifying function in Drosophila, MANF was overexpressed in hemocytes in vivo or hemocytes in culture were treated with hrMANF protein. Both conditions were sufficient to significantly increase the proportion of lamellocytes in hemocyte smears, as detected by Atilla expression (FIG. 4A-C). This correlated with a significant decrease in the proportion of cells expressing GFP driven by Hml::Gal4 (FIG. 4C, right) and a significant decrease in hml transcripts (FIG. 4D). This suggests that MANF can act in a non-cell autonomous manner to promote hemocyte activation.

The Drosophila homolog of mammalian arginase1, arginase (arg) is expressed in fly hemocytes, suggesting that these cells may be able to acquire phenotypes similar to M2 polarization. Strikingly, MANF over-expression significantly increased Arg expression (FIG. 4E-F). Arginase and MANF protein expression almost completely overlapped in resting conditions as well as after MANF overexpression (FIG. 4F). In wild type larvae, MANF/Arg expressing hemocytes account for about 50% of the hemocyte population and consistent with transcriptional induction of Arg (FIG. 4E), about 80% of the hemocyte population expressed Arg after MANF overexpression, paralleling the increase in MANF+ hemocytes (FIG. 4F). These effects are similar to the effects of MANF in mouse innate immune cells, and support the idea that MANF has a conserved immune modulatory function that promotes activation of hemocytes/microglia/macrophages into phenotypes associated with tissue repair.

### Example 6: MANF Immune Modulatory Activity is Required for Retinal Protection

To test the requirement of MANF's immune modulatory function for retinal repair, retinal tissue preservation was assessed in conditions in which hemocytes express and secrete high levels of MANF, but are unable to be activated in response to this signal.

Materials and Methods

Fly lines used are as described above.

Immunohistochemistry performed as described above.

Over-expression or treatment with hrMANF performed as described above.

qRT-PCR performed as described above.

Western Blot performed as described above.

Results

MANF contains a highly conserved RTDL C-terminal amino acid sequence, which was shown to interact with Kdel Receptors (KdelRs) in human cells. KdelR1 and KdelR2 promote MANF intracellular accumulation, and the interaction between KdelRs and MANF at the cell surface can be required for recruitment of extracellular MANF to the plasma membrane and its downstream effects. The Drosophila genome contains one homolog of mammalian KdelRs. Whether the function of KdelR in regulating intracellular retention of MANF in Drosophila is conserved was tested. Indeed, knock down of KdelR in hemocytes (FIG. 5A) resulted in a significant induction of MANF transcripts and the detection of MANF protein in the hemolymph (FIGS. 5A and B), suggesting that KdelR-depleted hemocytes secrete high levels of MANF.

Based on the observation that MANF can promote hemocyte activation in a non-cell autonomous manner, it was hypothesized that the KdelR-mediated recruitment of MANF to the plasma membrane may be conserved in Drosophila and required for MANF-induced hemocyte activation. Supporting this idea, it was found that Arg expression and lamellocyte formation were significantly impaired in hemocytes in which KdelR was knocked down even after MANF over-expression or treatment with hrMANF (FIG. 5C-F). This observation shows that hemocyte-specific KdelR knock down is an efficient way to impair MANF-mediated hemocyte activation despite high MANF secretion (FIG. 5G).

Thus, the KdelR knock down conditions were used to test the requirement of hemocyte activation for the cytoprotective function of MANF. UV-induced tissue loss was significantly enhanced after KdelR knock down in hemocytes, and could not be rescued by MANF over-expression (FIG. 5H-I). This finding suggests that previously reported direct anti-apoptotic function of MANF observed in cell culture is not the only mechanism through which MANF protects tissues from degeneration in live organisms, highlighting the importance of tissue interactions and anti-inflammatory environments for tissue repair.

### Example 7: MANF Promotes Cell Integration in the Mammalian Retina

The cytoprotective and immune modulatory activities of MANF described above introduce it as a good candidate to be used as co-adjuvant in retinal regenerative therapies and to improve cell engraftment in the retina after transplantation. Retinal repair by transplantation of mouse and human photoreceptor precursors can restore vision in mouse models of retinal degeneration with efficiency depending on the ontogenetic stage of donor cells and on the degree and status of the degenerative microenvironment. Particularly, the presence of classical activated macrophages within the retinal tissue negatively correlates with integration efficiency. Therefore, it was hypothesized that combining the neuroprotective and immune modulatory functions of MANF could be an efficient way to enhance integration efficiency, by acting both to promote the survival of transplanted cells and halting the negative impact of pro-inflammatory environments associated with degenerating retinas. This hypothesis was tested in this Example.

Materials and Methods

Mice

Nrl-EGFP transgenic mice were purchased from The Jackson Laboratory (JAX, stock nr. 021232) and are genetically tagged to express the green fluorescent protein (GFP) gene driven by the neural retina-specific leucine (Nrl) zipper promoter. The Nrl gene encodes a basic motif-leucine zipper transcription factor that is a necessary regulator of photoreceptor development and is required for the development of rod photoreceptors. Thus, the rod photoreceptors of donor Nrl-EGFP mice can be traced in the host retina after transplantation by detecting the presence of GFP protein.

Preparation of Nrl-GFP Cells for Sub-Retinal Injection

Four retinas of Nrl-EGFP mice were dissected in chilled 1× Hank's Balanced Salt Solution (HBSS) solution. For dissociation, retinas were incubated with 1 mL of Accutase (Global Cell Solutions, Charlottesville, Va.) at 37° C. for 30 minutes, and mechanically dissociated by pipetting every 10 minutes. Accutase was inactivated with 200 μL of Fetal Bovine Serum (FBS, J R Scientific, Woodland, Calif.) and the cell suspension was centrifuged at 1500 rpm for 3 minutes. The supernatant was removed, and the cell pellet was suspended in 24 μL of neural stem cell culture medium (DMEM/F-12 containing 1% Sodium Pyruvate, 1% of HEPES, 1% of Essential amino acid, 2% glucose, 1% Sodium Bicarbonate, 1% Penicillin/Streptomycin and 1% N2 supplement) with a supplement of 1% FBS. The cell suspension was split into two 10 μL aliquots and was supplemented with either 2 μg (in 2 μL volume) of hrMANF or with 2 μL of PBS immediately before subretinal injection.

Sub-Retinal Injection

Dissociated GFP-expressing mouse retinal cells from the Nrl-GFP mice were transplanted into the subretinal space of recipient mice using the trans-corneal subretinal injection method (FIG. 6A). Eyes were collected one week after transplant and stained for GFP, MANF and CD11b expression by Immunohistochemistry (IHC) as described below.

Procedure:

Following testing the depth of anesthesia, a small hole was made on the cornea using a 30 Gauge needle. A pulled graduated glass pipet (Wiretrol II, 5-0000-2005, Durmmond Scientific Company) containing 2 μL of cell suspension (7.5×10⁵ cells) was inserted into the anterior chamber of the eye through the hole, advanced gently across the iris until it reached the subretinal space of the eye. The cells were then slowly injected into the subretinal space using a wire plunger.

Quantification of Cell Integration in Sub-Retinal Injections

For all experiments, successful subretinal injections were determined by detecting the presence of GFP expressing cells in the subretinal space. For eyes with successful injections, retinas were serially sectioned in about 50 slides with 3 sections/slides and every 5th slide was stained with an antibody against GFP and DAPI. The number of integrated cells was quantified in all sections observed under the microscope. Cells were considered integrated if they were present within two rows of the host ONL as defined by DAPI staining. The total number of integrated cells/eye was extrapolated from the total number of slides/eye. Quantifications include 4-9 eyes.

Quantification MANF-Expressing Innate Immune Cells in Transplant Sites

The number of MANF+CD11b+ innate immune cells in integration sites was performed in 10 fields (1024×1024 px) per condition, from confocal captures of retinal sections stained with GFP, CD11b and MANF. All fields quantified contained GFP+ cells in the subretinal space and MANF+CD11b+ innate immune cells were quantified within the ONL/INL and adjacent plexiform layers.

Results

Expression of MANF in microglia and/or macrophages at sites of integration of donor Nrl-GFP photoreceptors in undamaged retinas (FIG. 6A-B) was assessed. Indeed, immune cells at or close to sites of integration expressed MANF and their presence positively correlated with integration efficiency (FIG. 6C). Very poor or no integration of photoreceptors at sites lacking MANF+ immune cells was found, despite the presence of donor photoreceptors in the subretinal space.

To directly test if MANF supplementation can improve integration efficiency, photoreceptor integration from mouse Nrl-GFP donors one week after subretinal delivery (FIG. 6A) was compared with MANF or vehicle supplementation. First, the ability of MANF to enhance integration of mature (P21) donor photoreceptor cells was evaluated in wild type retinas, an ontogenic donor stage which usually fails to produce efficient integration. This failure can be, at least in part, due to increased susceptibility of mature photoreceptors to undergo apoptosis, and based on the data discussed above, this may be suppressed by MANF. Indeed, MANF supplementation could significantly improve the integration efficiency of P21 donor photoreceptors (FIG. 6D).

Finally, the ability of MANF to enhance integration was tested in degenerating retinas. As reported in other forms of retinal degeneration, photoreceptor integration efficiency was reduced in Crx^{tvrm65} retinas. Importantly, using MANF as a co-adjuvant, integration efficiency could be significantly enhanced to match that of wild type eyes (FIG. 6E).

Discussion

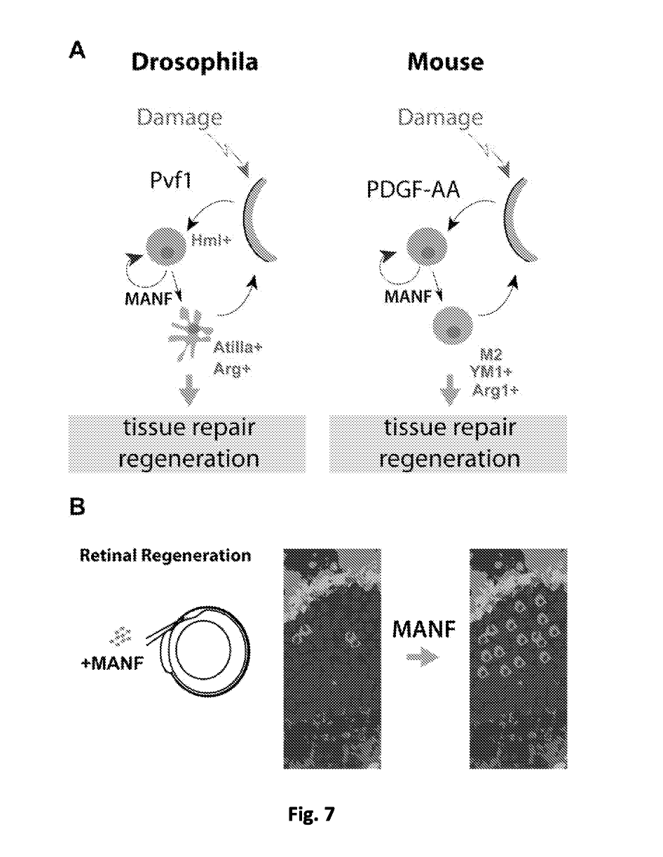

The results of Examples 1-7 identify MANF as an evolutionarily conserved immune modulator that plays a critical role in the regulatory network mediating tissue repair in the retina (FIG. 7A). The ability of MANF to increase regenerative success in the mouse retina highlights the promise of modulating the immune environment as a strategy to improve regenerative therapies (FIG. 7B).

Studies of tissues where regeneration is sustained endogenously by resident stem cells support the usefulness of immunomodulation for regenerative medicine. In skeletal muscle, type-2 innate signals (cytokine signals that favor M2 polarization) promote myogenesis and facilitate regeneration. In the CNS, in turn, immune cells can modulate neural progenitor function to facilitate neurogenesis, promote axonal regeneration and remyelination, and can play a central role in delivering anti-inflammatory signals. The results presented in this example strongly support the use of immunomodulation of the recipient environment as an efficient way to enhance the efficacy of cell replacement therapies because the results show that harnessing an endogenous, evolutionarily conserved, immune modulatory mechanism can enhance the integration efficiency of photoreceptors transplanted into damaged retinas.

Direct and indirect role of MANF in neuroprotection.

Several factors have previously been proposed to impact the efficiency of retinal repair. These include a number of neurotrophic factors, such as members of the FGF family, as well as BDNF, GDNF, IGF-1 and CNTF, which can protect against retinal degeneration. CNTF has been most widely studied and is currently in clinical trials for delivery using encapsulated cells. Most of these factors directly prevent photoreceptor apoptosis by stimulating AKT (IGFs and FGFs) or MAPK (CNTF) signaling pathways, which have an evolutionarily conserved anti-apoptotic function in neurons.

MANF has also been described as a neurotrophic factor, as it can rescue apoptotic neurons in culture and, together with its mammalian paralog CDNF, has been shown to prevent and rescue neuronal death in murine models of Parkinson disease. In the retina, MANF may also exert a direct neuroprotective effect, yet these data suggest a more expansive role: since MANF did not promote tissue repair in flies in which the hemocyte response was selectively ablated, we propose that the role of MANF in promoting alternative (M2) activation of innate immune cells is central to its function in tissue repair. Clinically, MANF may thus have a distinct advantage over previously described neurotrophic factors in both improving survival of transplanted cells directly, as well as in promoting a microenvironment supportive of local repair and integration. Because integration efficiency correlates with the extent of vision restoration, MANF supplementation will have an important impact in clinical settings.

Modulation of the immune environment and tissue repair.

The identification of an evolutionarily conserved, cytokine-like immune modulatory activity for MANF, and the importance of this activity for tissue repair, provides interesting new insight into the role of immune cells in tissue repair. In Drosophila, hemocyte activation was previously found to be important for tissue repair after damage, yet the molecular determinants and cellular phenotypes associated with this activation remained unclear. The data presented reveal evolutionary conservation of a PvR/MANF-signaling axis in the interaction between damaged tissue and immune cells, and in promoting tissue repair and homeostasis. Interestingly, a recent report has identified PDGF-AA signaling from senescent cells as an enhancer of tissue repair during epidermal wound closure, suggesting that similar mechanisms may be more broadly used.

Hemocyte states resembling M1/M2 macrophage activation in mammals have not previously been described in flies, yet the association of atilla and arginase expression (important makers of M2-activation in mammals) with improved tissue repair suggests that a protective, pro-repair, M2-like hemocyte activation state exists. Furthermore, a recent report shows that loss of MANF results in the infiltration of pupal brains with cells resembling hemocytes with high Rel/NFkB activity, potentially representing pro-inflammatory, M1-like phenotypes. This supports the idea that the mechanisms of innate immune cell polarization may be evolutionarily conserved and that Drosophila may be used as a tool to further characterize this homeostatic mechanism.

The findings presented here show that mammalian MANF, similar to fly MANF, also increases the presence of M2 macrophages/microglia in damaged tissue. Recent studies in MANF mutant mice, in turn, show that loss of MANF leads to beta cell loss in the pancreas. Beta cell loss is a common characteristic of conditions associated with chronic inflammation, and the data presented here indicate that MANF may be broadly required in various contexts to aid conversion of M1 pro-inflammatory macrophages into pro-repair anti-inflammatory M2 macrophages. This view is supported by the fact that MANF (and its close relative CDNF) exerts protective effects in vivo in murine models of Parkinson's disease. Since neurotoxic inflammation has been implicated in Parkinson's disease, it is thus possible that the protective effects of MANF in this context are, at least in part, mediated by immunomodulation. Indeed, the MANF paralog CDNF may have an anti-inflammatory function in murine models of Parkinson's disease and in nerve regeneration after spinal cord injury. The results presented here support a role for MANF in resolving inflammation and promoting tissue repair not only in the retina and brain but also in other tissues.

The evolutionary conservation of MANF, as well as its broad damage-stimulated expression, support a wide-ranging role in tissue repair and regeneration processes. The data presented support such a role and highlight its usefulness for regenerative medicine.

### Example 8: Immune System Modulation with MANF

Stem cell based therapies have been shown to hold real promise in the treatment of degenerative diseases of the retina. However, the efficiency of such strategies is still considerably low. Tissue repair mechanisms are conserved at the organism level and enhance the regenerative process. It was hypothesized that promoting tissue repair may also enhance the efficiency of cell engraftment in the retina. Key components of the retinal repair network have been identified in Drosophila involving interactions between the damaged retina and hemocytes. Drosophila were used to identify hemocyte derived factors that can promote tissue repair in the retina and the conservation of their function in the mammalian retina was tested. The work focused on Mesencephalic Astrocyte-derived Neurotrophic Factor (MANF).

Materials and Methods

UV induced retinal damage in flies and light induced retinal damage in mouse were used as model systems to test the effects of MANF. MANF was overexpressed in flies using the UAS/Gal4 system. In mice, recombinant protein was delivered by intravitreal injection.

Results

MANF was identified using RNAseq as a hemocyte derived protein in Drosophila that can promote tissue repair in the fly retina. This example shows that MANF is expressed in hemocytes of Drosophila larvae, it is secreted to the hemolymph and induced in response to stress in a Pvf-1/PvR dependent manner. Hemocyte specific MANF expression is sufficient to reduce tissue loss after UV and genetically-induced photoreceptor apoptosis. Moreover, stress induced MANF results in changes in the hemocyte population correlating with increased lamellocyte differentiation. We have tested the conservation of the pathway in mammalian retinal repair. As in flies, MANF is induced in microglia/macrophages invading the retina following light damage and this correlates with reduced tissue loss. Importantly, intravitreal delivery of MANF recombinant protein is sufficient to limit cell death following light damage and promotes alterations in macrophages/microglia.

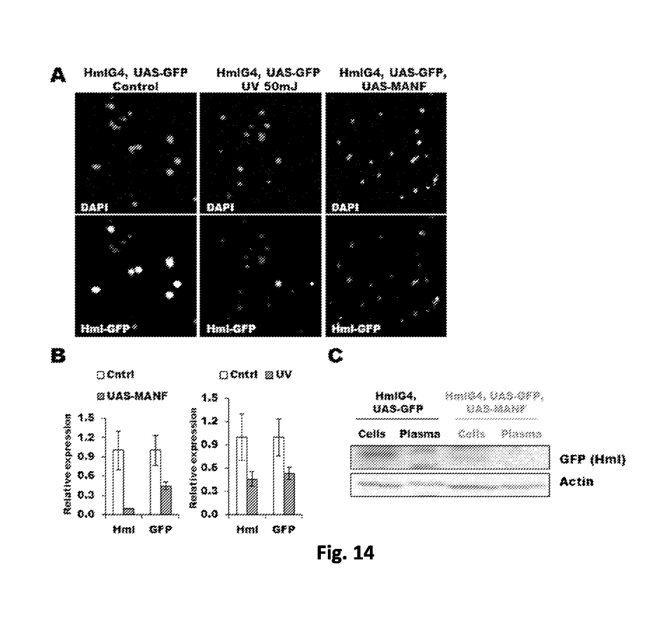

FIG. 14 shows that overexpression of MANF in Drosophila hemocytes mimics the stress response to UV exposure. Both overexpression of MANF and UV induced stress results in down regulation of plasmatocyte marker Hml. This was, to the inventors' knowledge, the first showing that MANF can modulate the immune system.

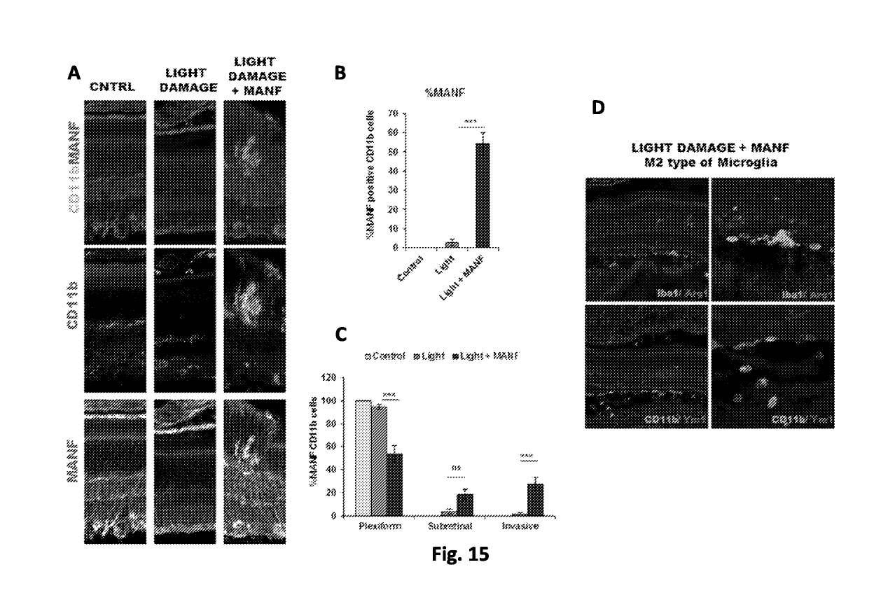

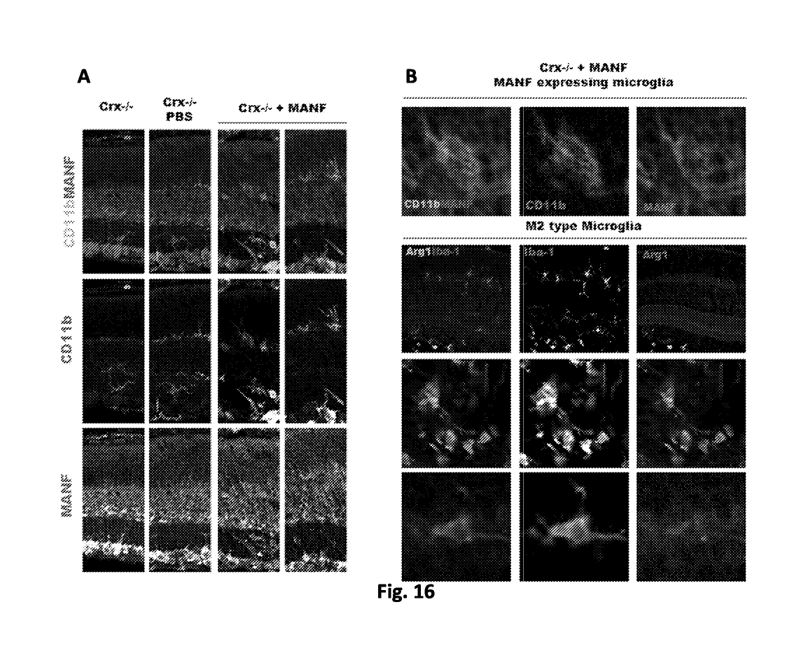

FIGS. 15 and 16 show that exogenous delivery of MANF changes the immune environment in two mouse models of retinal degeneration. FIG. 15 shows an increase in invasive MANF expressing amoeboid shape microglia/macrophages M2 type of microglia when MANF in injected prior to light induced retinal damage. FIG. 16 shows an increase in invasive MANF expressing amoeboid shape microglia/macrophases in a genetic model of retinal degeneration (CRX1−/−) following MANF injection. In both cases, M2 (alternative activated/tissue protective) macrophage markers can be detected.

Conclusions:

MANF is a conserved neuroprotective factor in the retina. MANF acts as an immune-modifying factor to limit cell loss following acute damage. This work will serve as a proof of concept to the use of tissue repair promoting factors as co-adjutants in stem cell regenerative therapies.

### Example 9: MANF Treatment Induced the Expression of Markers of M2 Activation in Cultured Macrophages

This examples shows that treatment with exogenous MANF results in MD marker expression in a macrophage cell line.

Materials and Methods

Abelson murine leukemia virus transformed RAW 264.7 (ATCC TIB-71™) macrophages were cultured according to known methods for 3, 6 and 16 h in DMEM medium supplemented with 10% Fetal Bovine serum and MANF (10 μg/μl), in the presence or absence of LPS (Lipopolysaccharide, pro-inflammatory agent). RT-PCR was performed as described above.

Results

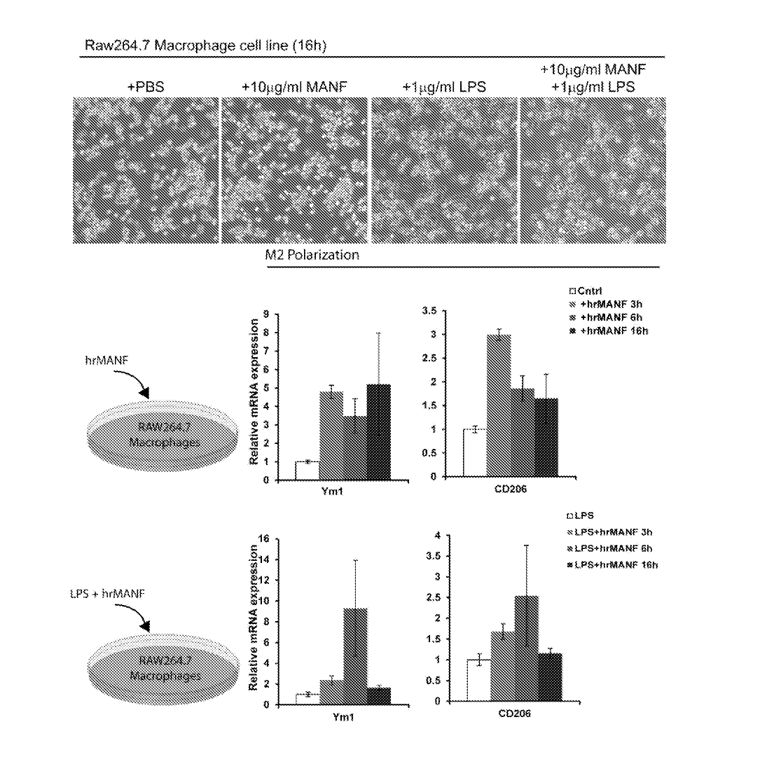

Bright filed images collected at 16 h (FIG. 17, top panel), show a partial recovery in morphology of LPS treated cells when MANF is co-supplemented (compare LPS vs. LPS+ MANF). RNA samples were collected at the designated times and relative mRNA levels of two markers of M2 (alternative) macrophage polarization (Ym1 and CD206) were quantified. In the absence (FIG. 17, top graph) and presence (FIG. 17, bottom graph) of a pro-inflammatory agent, MANF treatment induced the expression of markers of M2 activation in Raw264.7 Macrophages. N=3 for all time points and treatments.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

Each of the references below are incorporated by reference for all purposes.
Abrams, E. W., Cheng, Y. L., and Andrew, D. J. (2013). Drosophila KDEL receptor function in the embryonic salivary gland and epidermis. PloS one 8, e77618.
Arnold, L., Henry, A., Poron, F., Baba-Amer, Y., van Rooijen, N., Plonquet, A., Gherardi, R. K., and Chazaud, B. (2007). Inflammatory monocytes recruited after skeletal muscle injury switch into antiinflammatory macrophages to support myogenesis. The Journal of experimental medicine 204, 1057-1069.
Aurora, A. B., and Olson, E. N. (2014). Immune modulation of stem cells and regeneration. Cell stem cell 15, 14-25.
Azadi, S., Johnson, L. E., Paquet-Durand, F., Perez, M. T., Zhang, Y., Ekstrom, P. A., and van Veen, T. (2007). CNTF+BDNF treatment and neuroprotective pathways in the rd1 mouse retina. Brain research 1129, 116-129.
Babcock, D. T., Brock, A. R., Fish, G. S., Wang, Y., Perrin, L., Krasnow, M. A., and Galko, M. J. (2008). Circulating blood cells function as a surveillance system for damaged tissue in Drosophila larvae. Proceedings of the National Academy of Sciences of the United States of America 105, 10017-10022.
Barber, A. C., Hippert, C., Duran, Y., West, E. L., Bainbridge, J. W., Warre-Cornish, K., Luhmann, U F, Lakowski, J., Sowden, J. C., Ali, R. R., et al. (2013). Repair of the degenerate retina by photoreceptor transplantation. Proceedings of the National Academy of Sciences of the United States of America 110, 354-359.
Belacortu, Y., and Paricio, N. (2011). Drosophila as a model of wound healing and tissue regeneration in vertebrates. Developmental dynamics: an official publication of the American Association of Anatomists 240, 2379-2404.
Brock, A. R., Babcock, D. T., and Galko, M. J. (2008). Active cop, passive cop: developmental stage-specific modes of wound-induced blood cell recruitment in Drosophila. Fly 2, 303-305.
Cao, X., Shen, D., Patel, M. M., Tuo, J., Johnson, T. M., Olsen, T. W., and Chan, C. C. (2011). Macrophage polarization in the maculae of age-related macular degeneration: a pilot study. Pathology international 61, 528-535.
Chen, M., Luo, C., Penalva, R., and Xu, H. (2013). Paraquat-induced retinal degeneration is exaggerated in CX3CR1-deficient mice and is associated with increased retinal inflammation. Invest Ophthalmol Vis Sci 54, 682-690.
Chen, Y. C., Sundvik, M., Rozov, S., Priyadarshini, M., and Panula, P. (2012). MANF regulates dopaminergic neuron development in larval zebrafish. Developmental biology 370, 237-249.
Combadiere, C., Feumi, C., Raoul, W., Keller, N., Rodero, M., Pezard, A., Lavalette, S., Houssier, M., Jonet, L., Picard, E., et al. (2007). CX3CR1-dependent subretinal microglia cell accumulation is associated with cardinal features of age-related macular degeneration. The Journal of clinical investigation 117, 2920-2928.
Cruz-Guilloty, F., Saeed, A. M., Echegaray, J. J., Duffort, S., Ballmick, A., Tan, Y., Betancourt, M., Viteri, E., Ramkhellawan, G. C., Ewald, E., et al. (2013). Infiltration of proinflammatory ml macrophages into the outer retina precedes damage in a mouse model of age-related macular degeneration. International journal of inflammation 2013, 503725.
Demaria, M., Ohtani, N., Youssef, S. A., Rodier, F., Toussaint, W., Mitchell, J. R., Laberge, R. M., Vijg, J., Van Steeg, H., Dolle, M. E., et al. (2014). An Essential Role for Senescent Cells in Optimal Wound Healing through Secretion of PDGF-AA. Developmental cell 31, 722-733.
Dilly, A. K., and Rajala, R. V. (2008). Insulin growth factor 1 receptor/PI3K/AKT survival pathway in outer segment membranes of rod photoreceptors. Invest Ophthalmol Vis Sci 49, 4765-4773.
Duchek, P., Somogyi, K., Jekely, G., Beccari, S., and Rorth, P. (2001). Guidance of cell migration by the Drosophila PDGF/VEGF receptor. Cell 107, 17-26.
Dziennis, S., Van Etten, R. A., Pahl, H. L., Morris, D. L., Rothstein, T. L., Blosch, C. M., Perlmutter, R. M., and Tenen, D. G. (1995). The CD11b promoter directs high-level expression of reporter genes in macrophages in transgenic mice. Blood 85, 319-329.
Dzik, J. M. (2014). Evolutionary roots of arginase expression and regulation. Frontiers in immunology 5, 544.
Forbes, S. J., and Rosenthal, N. (2014). Preparing the ground for tissue regeneration: from mechanism to therapy. Nature medicine 20, 857-869.
Fox, I. J., Daley, G. Q., Goldman, S. A., Huard, J., Kamp, T. J., and Trucco, M. (2014). Stem cell therapy. Use of differentiated pluripotent stem cells as replacement therapy for treating disease. Science 345, 1247391.
Franceschi, C., and Campisi, J. (2014). Chronic inflammation (inflammaging) and its potential contribution to age-associated diseases. The journals of gerontology Series A, Biological sciences and medical sciences 69 Suppl 1, S4-9.
Freeman, M. (1996). Reiterative use of the EGF receptor triggers differentiation of all cell types in the Drosophila eye. Cell 87, 651-660.
Gordon, S. (2003). Alternative activation of macrophages. Nature reviews Immunology 3, 23-35.
Green, E. S., Rendahl, K. G., Zhou, S., Ladner, M., Coyne, M., Srivastava, R., Manning, W. C., and Flannery, J. G. (2001). Two animal models of retinal degeneration are rescued by recombinant adeno-associated virus-mediated production of FGF-5 and FGF-18. Molecular therapy: the journal of the American Society of Gene Therapy 3, 507-515.
Gupta, N., Brown, K. E., and Milam, A. H. (2003). Activated microglia in human retinitis pigmentosa, late-onset retinal degeneration, and age-related macular degeneration. Experimental eye research 76, 463-471.
Gust, J., and Reh, T. A. (2011). Adult donor rod photoreceptors integrate into the mature mouse retina. Investigative ophthalmology & visual science 52, 5266-5272.
Hao, W., Wenzel, A., Obin, M. S., Chen, C. K., Brill, E., Krasnoperova, N. V., Eversole-Cire, P., Kleyner, Y., Taylor, A., Simon, M. I., et al. (2002). Evidence for two apoptotic pathways in light-induced retinal degeneration. Nature genetics 32, 254-260.
Heino, T. I., Karpanen, T., Wahlstrom, G., Pulkkinen, M., Eriksson, U., Alitalo, K., and Roos, C. (2001). The Drosophila VEGF receptor homolog is expressed in hemocytes. Mechanisms of development 109, 69-77.
Henderson, M. J., Richie, C. T., Airavaara, M., Wang, Y., and Harvey, B. K. (2013). Mesencephalic astrocyte-derived neurotrophic factor (MANF) secretion and cell surface binding are modulated by KDEL receptors. The Journal of biological chemistry 288, 4209-4225.
Heredia, J. E., Mukundan, L., Chen, F. M., Mueller, A. A., Deo, R. C., Locksley, R. M., Rando, T. A., and Chawla, A. (2013). Type 2 innate signals stimulate fibro/adipogenic progenitors to facilitate muscle regeneration. Cell 153, 376-388.
Hirsch, E. C., and Hunot, S. (2009). Neuroinflammation in Parkinson's disease: a target for neuroprotection? The Lancet Neurology 8, 382-397.
Jasper, H., and Bohmann, D. (2002). Drosophila innate immunity: a genomic view of pathogen defense. Molecular cell 10, 967-969.
Jassim, O. W., Fink, J. L., and Cagan, R. L. (2003). Dmp53 protects the Drosophila retina during a developmentally regulated DNA damage response. The EMBO journal 22, 5622-5632.
Kaneko, H., Nishiguchi, K. M., Nakamura, M., Kachi, S., and Terasaki, H. (2008). Characteristics of bone marrow-derived microglia in the normal and injured retina. Investigative ophthalmology & visual science 49, 4162-4168.
Kang, M. J., and Ryoo, H. D. (2009). Suppression of retinal degeneration in Drosophila by stimulation of ER-associated degradation. Proceedings of the National Academy of Sciences of the United States of America 106, 17043-17048.
Karlstetter, M., Ebert, S., and Langmann, T. (2010). Microglia in the healthy and degenerating retina: insights from novel mouse models. Immunobiology 215, 685-691.
Karpac, J., Younger, A., and Jasper, H. (2011). Dynamic coordination of innate immune signaling and insulin signaling regulates systemic responses to localized DNA damage. Developmental cell 20, 841-854.
Kassen, S. C., Thummel, R., Campochiaro, L. A., Harding, M. J., Bennett, N. A., and Hyde, D. R. (2009). CNTF induces photoreceptor neuroprotection and Muller glial cell proliferation through two different signaling pathways in the adult zebrafish retina. Experimental eye research 88, 1051-1064.
Keeler, C. (1966). Retinal degeneration in the mouse is rodless retina. The Journal of heredity 57, 47-50.
Kelsey, E. M., Luo, X., Bruckner, K., and Jasper, H. (2012). Schnurri regulates hemocyte function to promote tissue recovery after DNA damage. Journal of cell science 125, 1393-1400.
Kigerl, K. A., Gensel, J. C., Ankeny, D. P., Alexander, J. K., Donnelly, D. J., and Popovich, P. G. (2009). Identification of two distinct macrophage subsets with divergent effects causing either neurotoxicity or regeneration in the injured mouse spinal cord. The Journal of neuroscience: the official journal of the Society for Neuroscience 29, 13435-13444.
Kokaia, Z., Martino, G., Schwartz, M., and Lindvall, O. (2012). Cross-talk between neural stem cells and immune cells: the key to better brain repair? Nature neuroscience 15, 1078-1087.
Kurimoto, T., Yin, Y., Habboub, G., Gilbert, H. Y., Li, Y., Nakao, S., Hafezi-Moghadam, A., and Benowitz, L. I. (2013). Neutrophils express oncomodulin and promote optic nerve regeneration. The Journal of neuroscience: the official journal of the Society for Neuroscience 33, 14816-14824.
Lamba, D. A., Gust, J., and Reh, T. A. (2009). Transplantation of human embryonic stem cell-derived photoreceptors restores some visual function in Crx-deficient mice. Cell stem cell 4, 73-79.
Lau, D., and Flannery, J. (2003). Viral-mediated FGF-2 treatment of the constant light damage model of photoreceptor degeneration. Documenta ophthalmologica Advances in ophthalmology 106, 89-98.
Lawrence, J. M., Keegan, D. J., Muir, E. M., Coffey, P. J., Rogers, J. H., Wilby, M. J., Fawcett, J. W., and Lund, R. D. (2004). Transplantation of Schwann cell line clones secreting GDNF or BDNF into the retinas of dystrophic Royal College of Surgeons rats. Invest Ophthalmol Vis Sci 45, 267-274.
Lindahl, M., Danilova, T., Palm, E., Lindholm, P., Voikar, V., Hakonen, E., Ustinov, J., Andressoo, J. O., Harvey, B. K., Otonkoski, T., et al. (2014). MANF is indispensable for the proliferation and survival of pancreatic beta cells. Cell reports 7, 366-375.
Lindholm, P., Peranen, J., Andressoo, J. O., Kalkkinen, N., Kokaia, Z., Lindvall, O., Timmusk, T., and Saarma, M. (2008). MANF is widely expressed in mammalian tissues and differently regulated after ischemic and epileptic insults in rodent brain. Molecular and cellular neurosciences 39, 356-371.
Lindholm, P., and Saarma, M. (2010). Novel CDNF/MANF family of neurotrophic factors. Developmental neurobiology 70, 360-371.
Lindholm, P., Voutilainen, M. H., Lauren, J., Peranen, J., Leppanen, V. M., Andressoo, J. O., Lindahl, M., Janhunen, S., Kalkkinen, N., Timmusk, T., et al. (2007). Novel neurotrophic factor CDNF protects and rescues midbrain dopamine neurons in vivo. Nature 448, 73-77.
London, A., Cohen, M., and Schwartz, M. (2013). Microglia and monocyte-derived macrophages: functionally distinct populations that act in concert in CNS plasticity and repair. Frontiers in cellular neuroscience 7, 34.
London, A., Itskovich, E., Benhar, I., Kalchenko, V., Mack, M., Jung, S., and Schwartz, M. (2011). Neuroprotection and progenitor cell renewal in the injured adult murine retina requires healing monocyte-derived macrophages. The Journal of experimental medicine 208, 23-39.
Lucin, K. M., and Wyss-Coray, T. (2009). Immune activation in brain aging and neurodegeneration: too much or too little? Neuron 64, 110-122.
Luo, X., Puig, O., Hyun, J., Bohmann, D., and Jasper, H. (2007). Foxo and Fos regulate the decision between cell death and survival in response to UV irradiation. The EMBO journal 26, 380-390.
MacLaren, R. E., Pearson, R. A., MacNeil, A., Douglas, R. H., Salt, T. E., Akimoto, M., Swaroop, A., Sowden, J. C., and Ali, R. R. (2006). Retinal repair by transplantation of photoreceptor precursors. Nature 444, 203-207.
Markus, R., Kurucz, E., Rus, F., and Ando, I. (2005). Sterile wounding is a minimal and sufficient trigger for a cellular immune response in Drosophila melanogaster. Immunology letters 101, 108-111.
Mills, C. D. (2012). M1 and M2 Macrophages: Oracles of Health and Disease. Critical reviews in immunology 32, 463-488.
Miron, V. E., Boyd, A., Zhao, J. W., Yuen, T. J., Ruckh, J. M., Shadrach, J. L., van Wijngaarden, P., Wagers, A. J., Williams, A., Franklin, R J, et al. (2013). M2 microglia and macrophages drive oligodendrocyte differentiation during CNS remyelination. Nature neuroscience 16, 1211-1218.
Nadella, R., Voutilainen, M. H., Saarma, M., Gonzalez-Barrios, J. A., Leon-Chavez, B. A., Duenas Jimenez, J. M., Duenas Jimenez, S. H., Escobedo, L., and Martinez-Fong, D. (2014). Transient transfection of human CDNF gene reduces the 6-hydroxydopamine-induced neuroinflammation in the rat substantia nigra. Journal of neuroinflammation 11, 209.
Neves, J., Demaria, M., Campisi, J., and Jasper, H. (2015). Of flies, mice and men: Evolutionarily conserved tissue damage responses and aging Developmental cell.
Novak, M. L., and Koh, T. J. (2013). Phenotypic transitions of macrophages orchestrate tissue repair. The American journal of pathology 183, 1352-1363.
Palgi, M., Lindstrom, R., Peranen, J., Piepponen, T. P., Saarma, M., and Heino, T. I. (2009). Evidence that DmMANF is an invertebrate neurotrophic factor supporting dopaminergic neurons. Proceedings of the National Academy of Sciences of the United States of America 106, 2429-2434.
Pastor-Pareja, J. C., Wu, M., and Xu, T. (2008). An innate immune response of blood cells to tumors and tissue damage in Drosophila. Disease models & mechanisms 1, 144-154; discussion 153.
Pearson, R. A., Hippert, C., Graca, A. B., and Barber, A. C. (2014). Photoreceptor replacement therapy: Challenges presented by the diseased recipient retinal environment. Visual neuroscience 31, 333-344.
Peng, B., Xiao, J., Wang, K., So, K. F., Tipoe, G. L., and Lin, B. (2014). Suppression of microglial activation is neuroprotective in a mouse model of human retinitis pigmentosa. The Journal of neuroscience: the official journal of the Society for Neuroscience 34, 8139-8150.
Petrova, P., Raibekas, A., Pevsner, J., Vigo, N., Anafi, M., Moore, M. K., Peaire, A. E., Shridhar, V., Smith, D. I., Kelly, J., et al. (2003). MANF: a new mesencephalic, astrocyte-derived neurotrophic factor with selectivity for dopaminergic neurons. Journal of molecular neuroscience: MN 20, 173-188.
Pitsouli, C., Apidianakis, Y., and Perrimon, N. (2009). Homeostasis in infected epithelia: stem cells take the lead. Cell host & microbe 6, 301-307.
Pittler, S. J., Keeler, C. E., Sidman, R. L., and Baehr, W. (1993). PCR analysis of DNA from 70-year-old sections of rodless retina demonstrates identity with the mouse rd defect. Proceedings of the National Academy of Sciences of the United States of America 90, 9616-9619.
Rapp, L. M., and Williams, T. P. (1980). The role of ocular pigmentation in protecting against retinal light damage. Vision research 20, 1127-1131.
Razzell, W., Wood, W., and Martin, P. (2011). Swatting flies: modelling wound healing and inflammation in Drosophila. Disease models & mechanisms 4, 569-574.
Reynolds, J., and Lamba, D. A. (2014). Human embryonic stem cell applications for retinal degenerations. Experimental eye research 123, 151-160.
Samson, M. L. (2000). Drosophila arginase is produced from a nonvital gene that contains the elav locus within its third intron. The Journal of biological chemistry 275, 31107-31114.
Sasahara, M., Otani, A., Oishi, A., Kojima, H., Yodoi, Y., Kameda, T., Nakamura, H., and Yoshimura, N. (2008). Activation of bone marrow-derived microglia promotes photoreceptor survival in inherited retinal degeneration. The American journal of pathology 172, 1693-1703.
Shechter, R., London, A., Varol, C., Raposo, C., Cusimano, M., Yovel, G., Rolls, A., Mack, M., Pluchino, S., Martino, G., et al. (2009). Infiltrating blood-derived macrophages are vital cells playing an anti-inflammatory role in recovery from spinal cord injury in mice. PLoS medicine 6, e1000113.
Sieving, P. A., Caruso, R. C., Tao, W., Coleman, H. R., Thompson, D. J., Fullmer, K. R., and Bush, R. A. (2006). Ciliary neurotrophic factor (CNTF) for human retinal degeneration: phase I trial of CNTF delivered by encapsulated cell intraocular implants. Proc Natl Acad Sci USA 103, 3896-3901.
Sinenko, S. A., and Mathey-Prevot, B. (2004). Increased expression of Drosophila tetraspanin, Tsp68C, suppresses the abnormal proliferation of ytr-deficient and Ras/Raf-activated hemocytes. Oncogene 23, 9120-9128.
Song, Z., Guan, B., Bergman, A., Nicholson, D. W., Thornberry, N. A., Peterson, E. P., and Steller, H. (2000). Biochemical and genetic interactions between Drosophila caspases and the proapoptotic genes rpr, hid, and grim. Molecular and cellular biology 20, 2907-2914.
Springer, T., Galfre, G., Secher, D. S., and Milstein, C. (1979). Mac-1: a macrophage differentiation antigen identified by monoclonal antibody. European journal of immunology 9, 301-306.
Stratoulias, V., and Heino, T. I. (2014). MANF silencing, immunity induction or autophagy trigger an unusual cell type in metamorphosing Drosophila brain. Cellular and molecular life sciences: CMLS.
Tadimalla, A., Belmont, P. J., Thuerauf, D. J., Glassy, M. S., Martindale, J. J., Gude, N., Sussman, M. A., and Glembotski, C. C. (2008). Mesencephalic astrocyte-derived neurotrophic factor is an ischemia-inducible secreted endoplasmic reticulum stress response protein in the heart. Circulation research 103, 1249-1258.
Tao, W., Wen, R., Goddard, M. B., Sherman, S. D., O'Rourke, P. J., Stabila, P. F., Bell, W. J., Dean, B. J., Kauper, K. A., Budz, V. A., et al. (2002). Encapsulated cell-based delivery of CNTF reduces photoreceptor degeneration in animal models of retinitis pigmentosa. Invest Ophthalmol Vis Sci 43, 3292-3298.
Tokusumi, T., Sorrentino, R. P., Russell, M., Ferrarese, R., Govind, S., and Schulz, R. A. (2009). Characterization of a lamellocyte transcriptional enhancer located within the misshapen gene of Drosophila melanogaster. PloS one 4, e6429.
Voutilainen, M. H., Back, S., Porsti, E., Toppinen, L., Lindgren, L., Lindholm, P., Peranen, J., Saarma, M., and Tuominen, R. K. (2009). Mesencephalic astrocyte-derived neurotrophic factor is neurorestorative in rat model of Parkinson's disease. The Journal of neuroscience: the official journal of the Society for Neuroscience 29, 9651-9659.
Wang, L., Kounatidis, I., and Ligoxygakis, P. (2013). Drosophila as a model to study the role of blood cells in inflammation, innate immunity and cancer. Frontiers in cellular and infection microbiology 3, 113.
Wenzel, A., Grimm, C., Marti, A., Kueng-Hitz, N., Hafezi, F., Niemeyer, G., and Reme, C. E. (2000). c-fos controls the “private pathway” of light-induced apoptosis of retinal photoreceptors. The Journal of neuroscience: the official journal of the Society for Neuroscience 20, 81-88.
West, E. L., Pearson, R. A., Barker, S. E., Luhmann, U. F., Maclaren, R. E., Barber, A. C., Duran, Y., Smith, A. J., Sowden, J. C., and Ali, R. R. (2010). Long-term survival of photoreceptors transplanted into the adult murine neural retina requires immune modulation. Stem Cells 28, 1997-2007.
Won, J., Shi, L. Y., Hicks, W., Wang, J., Hurd, R., Naggert, J. K., Chang, B., and Nishina, P. M. (2011). Mouse model resources for vision research. Journal of ophthalmology 2011, 391384.
Yin, Y., Henzl, M. T., Lorber, B., Nakazawa, T., Thomas, T. T., Jiang, F., Langer, R., and Benowitz, L. I. (2006). Oncomodulin is a macrophage-derived signal for axon regeneration in retinal ganglion cells. Nature neuroscience 9, 843-852.
Yoshida, N., Ikeda, Y., Notomi, S., Ishikawa, K., Murakami, Y., Hisatomi, T., Enaida, H., and Ishibashi, T. (2013). Laboratory evidence of sustained chronic inflammatory reaction in retinitis pigmentosa. Ophthalmology 120, e5-12.
Zeng, H. Y., Zhu, X. A., Zhang, C., Yang, L. P., Wu, L. M., and Tso, M. O. (2005). Identification of sequential events and factors associated with microglial activation, migration, and cytotoxicity in retinal degeneration in rd mice. Investigative ophthalmology & visual science 46, 2992-2999.
Zettervall, C. J., Anderl, I., Williams, M. J., Palmer, R., Kurucz, E., Ando, I., and Hultmark, D. (2004). A directed screen for genes involved in Drosophila blood cell activation. Proceedings of the National Academy of Sciences of the United States of America 101, 14192-14197.
Zhang, C., Shen, J. K., Lam, T. T., Zeng, H. Y., Chiang, S. K., Yang, F., and Tso, M. O. (2005). Activation of microglia and chemokines in light-induced retinal degeneration. Molecular vision 11, 887-895.
Zhao, H., Cheng, L., Du, X., Hou, Y., Liu, Y., Cui, Z., and Nie, L. (2014). Transplantation of Cerebral Dopamine Neurotrophic Factor Transducted BMSCs in Contusion Spinal Cord Injury of Rats: Promotion of Nerve Regeneration by Alleviating Neuroinflammation. Molecular neurobiology.
Akimoto, M., Cheng, H., Zhu, D., Brzezinski, J. A., Khanna, R., Filippova, E., Oh, E. C., Jing, Y., Linares, J. L., Brooks, M., et al. (2006). Targeting of GFP to newborn rods by Nrl promoter and temporal expression profiling of flow-sorted photoreceptors. Proc Natl Acad Sci USA 103, 3890-3895.
Chang, B., Hawes, N. L., Hurd, R. E., Davisson, M. T., Nusinowitz, S., and Heckenlively, J. R. (2002). Retinal degeneration mutants in the mouse. Vision research 42, 517-525.
Tiscornia, G., Singer, O., and Verma, I. M. (2006). Production and purification of lentiviral vectors. Nature protocols 1, 241-245.
Won, J., Shi, L. Y., Hicks, W., Wang, J., Hurd, R., Naggert, J. K., Chang, B., and Nishina, P. M. (2011). Mouse model resources for vision research. Journal of ophthalmology 2011, 391384.

1-74. (canceled)

75. A method of cell therapy comprising:
administering an effective amount of an immune modulating peptide comprising a MANF family protein, or fragment thereof, to a subject in need thereof; and
administering an effective amount of replacement cells to the subject.

76. The method of claim 75, wherein the immune modulating peptide is administered prior to the replacement cells.

77. The method of claim 75, wherein the immune modulating peptide is administered concurrently with the replacement cells.

78. The method of claim 75, wherein the replacement cells are contacted with the immune modulating peptide prior to administration.

79. The method of claim 75, wherein the MANF family protein is mesencephalic astrocyte-derived neurotrophic factor (MANF) or a fragment thereof.

80. The method of claim 75, wherein the peptide sequence of the immune modulating peptide comprises a sequence that has at least about 80% identity with SEQ ID NO:3.

81. The method of claim 75, wherein the immune modulating peptide has a length that is at least 80% the length of SEQ ID NO:3.

82. The method of claim 75, wherein the peptide sequence of the immune modulating peptide consists of a sequence listed in Table 3.

83. The method of claim 75, wherein the peptide sequence of the immune modulating peptide comprises SEQ ID NO:32 and SEQ ID NO:33.

84. The method of claim 82, wherein the immune modulating peptide is cell permeable.

85. The method of claim 75, wherein the MANF family protein is conserved dopamine neurotrophic factor (CDNF) or a fragment thereof.

86. The method of claim 75, wherein the peptide sequence of the immune modulating peptide comprises a sequence that has at least about 80% identity with SEQ ID NO:6.

87. The method of claim 86, wherein the immune modulating peptide has a length that is at least 80% the length of SEQ ID NO: 6.

88. The method of claim 75, wherein the peptide sequence of the immune modulating peptide consists of a sequence listed in Table 4.

89. The method of claim 75, wherein the peptide sequence of the immune modulating peptide comprises SEQ ID NO:34 and SEQ ID NO:35.

90. The method of claim 88, wherein the neuroprotective peptide is cell permeable.

91. The method of claim 75, wherein the replacement cells comprise adult stem cells, amniotic stem cells, cord blood stem cells, induced pluripotent stem cells, or a combination thereof.

92. The method of claim 75, wherein the replacement cells were produced from adult stem cells, amniotic stem cells, cord blood stem cells, induced pluripotent stem cells, or a combination thereof.

93. The method of claim 75, wherein the replacement cells comprise induced pluripotent stem cells.

94. The method of claim 75, wherein the replacement cells were produced from induced pluripotent stem cells.

95. The method of claim 75, wherein the replacement cells comprise autologous cells.

96. The method of claim 75, wherein the replacement cells comprise allogenic cells.

97. The method of claim 75, wherein the replacement cells were produced using a method that does not destroy an embryo capable of developing into a viable organism.

98. The method of claim 75, wherein the subject has a retinal disorder.

99. The method of claim 98, wherein the retinal disorder is macular degeneration, diabetic eye disease, age-related macular degeneration, branch retinal vein occlusion, central retinal vein occlusion, central retinal artery occlusion, central serous retinopathy, diabetic retinopathy, Fuchs' dystrophy, giant cell arteritis, glaucoma, hypertensive retinopathy, thyroid eye disease, iridocorneal endothelial syndrome, ischemic optic neuropathy, juvenile macular degeneration, macular edema, macular telangiectasia, marfan syndrome, optic neuritis, photokeratitis, retinitis pigmentosa, retinopathy of prematurity, stargardt disease, usher syndrome, Wolfram syndrome, or Leber Congenital Amaurosis.

100. The method of claim 98, wherein the replacement cells comprise retinal pigment epithelium cells.

101. The method of claim 98, wherein the replacement cells comprise retinal photoreceptor cells.

102. The method of claim 98, wherein the replacement cells are administered by intravitreal administration, intracameral administration, conjunctival administration, intracorneal administration, intraocular administration, ophthalmic administration, retrobulbar administration, subconjunctival administration, or by transplant.

103. The method of claim 98, wherein the immune modulating peptide is administered by intravitreal administration, intracameral administration, conjunctival administration, intracorneal administration, intraocular administration, ophthalmic administration, retrobulbar administration, or subconjunctival administration.


SEQUENCE LISTING (51 sequences)

SEQ ID NO: 1 - protein, 185 aa, Homo sapiens
MRRMRRMWATQGLAVALALSVLPGSRALRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 2 - protein, 179 aa, Homo sapiens [sequence omitted]

SEQ ID NO: 3 - protein, 158 aa, Homo sapiens
LRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 4 - protein, 159 aa, Artificial sequence
  Synthetic construct
MLRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 5 - protein, 187 aa, Homo sapiens
MWCASPVAVVAFCAGLLVSHPVLTQGQEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPKTEL

SEQ ID NO: 6 - protein, 161 aa, Homo sapiens
QEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPKTEL

SEQ ID NO: 7 - protein, 162 aa, Artificial sequence
  Synthetic construct
MQEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPKTEL

SEQ ID NO: 8 - protein, 40 aa, Homo sapiens
ILDDWGETCKGCAEKSDYIRKINELMPKYAPKAASARTDL

SEQ ID NO: 9 - protein, 39 aa, Homo sapiens
LDDWGETCKGCAEKSDYIRKINELMPKYAPKAASARTDL

SEQ ID NO: 10 - protein, 38 aa, Homo sapiens
DDWGETCKGCAEKSDYIRKINELMPKYAPKAASARTDL

SEQ ID NO: 11 - protein, 37 aa, Homo sapiens
DWGETCKGCAEKSDYIRKINELMPKYAPKAASARTDL

SEQ ID NO: 12 - protein, 36 aa, Homo sapiens
WGETCKGCAEKSDYIRKINELMPKYAPKAASARTDL

SEQ ID NO: 13 - protein, 35 aa, Homo sapiens
GETCKGCAEKSDYIRKINELMPKYAPKAASARTDL

SEQ ID NO: 14 - protein, 34 aa, Homo sapiens
ETCKGCAEKSDYIRKINELMPKYAPKAASARTDL

SEQ ID NO: 15 - protein, 33 aa, Homo sapiens
TCKGCAEKSDYIRKINELMPKYAPKAASARTDL

SEQ ID NO: 16 - protein, 32 aa, Homo sapiens
CKGCAEKSDYIRKINELMPKYAPKAASARTDL

SEQ ID NO: 17 - protein, 40 aa, Homo sapiens
KQILHSWGEECRACAEKTDYVNLIQELAPKYAATHPKTEL

SEQ ID NO: 18 - protein, 39 aa, Homo sapiens
QILHSWGEECRACAEKTDYVNLIQELAPKYAATHPKTEL

SEQ ID NO: 19 - protein, 38 aa, Homo sapiens
ILHSWGEECRACAEKTDYVNLIQELAPKYAATHPKTEL

SEQ ID NO: 20 - protein, 37 aa, Homo sapiens
LHSWGEECRACAEKTDYVNLIQELAPKYAATHPKTEL

SEQ ID NO: 21 - protein, 36 aa, Homo sapiens
HSWGEECRACAEKTDYVNLIQELAPKYAATHPKTEL

SEQ ID NO: 22 - protein, 35 aa, Homo sapiens
SWGEECRACAEKTDYVNLIQELAPKYAATHPKTEL

SEQ ID NO: 23 - protein, 34 aa, Homo sapiens
WGEECRACAEKTDYVNLIQELAPKYAATHPKTEL

SEQ ID NO: 24 - protein, 33 aa, Homo sapiens
GEECRACAEKTDYVNLIQELAPKYAATHPKTEL

SEQ ID NO: 25 - protein, 32 aa, Homo sapiens
EECRACAEKTDYVNLIQELAPKYAATHPKTEL

SEQ ID NO: 26 - protein, 31 aa, Homo sapiens
ECRACAEKTDYVNLIQELAPKYAATHPKTEL

SEQ ID NO: 27 - protein, 30 aa, Homo sapiens
CRACAEKTDYVNLIQELAPKYAATHPKTEL

SEQ ID NO: 28 - DNA, 993 bp, Homo sapiens
agctacggcgcgcggccgggacttggaggcggtgcggcgcggcgggtgcggttcagtcggtcggcggcggcagcggaggaggaggaggaggaggaggatgaggaggatgaggaggatgtgggccacgcaggggctggcggtggcgctggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcacggaccgatttgtagtctgctcaatctctgttgcacctgagggggaaaaaacagttcaactgcttactcccaaaacagcctttttgtaatttattttttaagtgggctcctgacaatactgtatcagatgtgaagcctggagctttcctgatgatgctggccctacagtacccccatgaggggattcccttccttctgttgctggtgtactctaggacttcaaagtgtgtctgggatttttttattaaagaaaaaaaatttctagctgtccttgcagaattatagtgaataccaaaatggggttttgccccaggaggctcctaaaaaaaaaaaaaaaaaaaaaaaaaaaaaaa

SEQ ID NO: 29 - DNA, 540 bp, Homo sapiens
atgtgggccacgcaggggctggcggtggcgctggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcacggaccgatttgtag

SEQ ID NO: 30 - DNA, 1330 bp, Homo sapiens
gcgcgggtggcggaggcgattgaagctgctggcccagcatgtggtgcgcgagcccagttgctgtggtggccttttgcgccgggcttttggtctctcacccggtgctgacgcagggccaggaggccggggggcggccaggggccgactgtgaagtatgtaaagaattcttgaaccgattctacaagtcactgatagacagaggagttaacttttcgctggacactatagagaaagaattgatcagtttttgcttggacaccaaaggaaaagaaaaccgcctgtgctattatctaggagccacaaaagacgcagccacaaagatcctaagtgaagtcactcgcccaatgagtgtgcatatgcctgcaatgaagatttgtgagaagctgaagaagttggatagccagatctgtgagctgaaatatgaaaaaacactggacttggcatcagttgacctgcggaagatgagagtggcagagctgaagcagatcctgcatagctggggggaggagtgcagggcctgtgcagaaaaaactgactatgtgaatctcattcaagagctggcccccaagtatgcagcgacacaccccaaaacagagctctgatctccaatgccagcacatttgtgacttgtaattagagagaaaagtgactctctaggatatggacatgttgattaaggataactgggaatgcatcatatttggtctcatgctttttgtgttggtattattcctcagaattttgttacgtgggtttatgagtgaaactaatactactgataacttacatttgcagtgtaccaaaagctaaaagttcctttctcataagtttcttggaatgactatgccagttttcattgcctgtctcctaaaagtgacctactgacaaattgatggagtaaattgattccaagaaagaagaaggcattcagagactcctctctggatgcaattttaaaatatattggactaaaacaaaagacacaacagtcagcttatctaatgcacaacttcaatcccaaatacagaatcaaaagtttttttcaagtgaattttctgttttcactctatattgtagctctctttggtatcagaaatggtcaggcaggagtactcgtttttcccattggaagaaaacccacttaaacctacttaaagaaaatagagagatcaagaaaccgtataagtatagccaatgtttggcaattatatcaacatttacattgttaattgaaccaagctcaaggtctttttcattccttacagtcacatatcagacaagtttttgatggaagattaaagcatattaaaaacactcataggcaacttgg

SEQ ID NO: 31 - DNA, 564 bp, Homo sapiens
atgtggtgcgcgagcccagttgctgtggtggccttttgcgccgggcttttggtctctcacccggtgctgacgcagggccaggaggccggggggcggccaggggccgactgtgaagtatgtaaagaattcttgaaccgattctacaagtcactgatagacagaggagttaacttttcgctggacactatagagaaagaattgatcagtttttgcttggacaccaaaggaaaagaaaaccgcctgtgctattatctaggagccacaaaagacgcagccacaaagatcctaagtgaagtcactcgcccaatgagtgtgcatatgcctgcaatgaagatttgtgagaagctgaagaagttggatagccagatctgtgagctgaaatatgaaaaaacactggacttggcatcagttgacctgcggaagatgagagtggcagagctgaagcagatcctgcatagctggggggaggagtgcagggcctgtgcagaaaaaactgactatgtgaatctcattcaagagctggcccccaagtatgcagcgacacaccccaaaacagagctctga

SEQ ID NO: 32 - protein, 4 aa, Artificial Sequence
  Conserved sequence in MANF
CKGC

SEQ ID NO: 33 - protein, 4 aa, Artificial Sequence
  KDEL-like C-terminal sequence
RTDL

SEQ ID NO: 34 - protein, 4 aa, Artificial Sequence
  Conserved sequence in CDNF
CRAC

SEQ ID NO: 35 - protein, 4 aa, Artificial Sequence
  KDEL-like C-terminal sequence
KTEL

SEQ ID NO: 36 - DNA, 22 bp, Mus musculus
caccagccactattgaagaaga

SEQ ID NO: 37 - DNA, 20 bp, Mus musculus
agcatcatctgtggctccaa

SEQ ID NO: 38 - DNA, 20 bp, Mus musculus
gctctggctcctagcaccat

SEQ ID NO: 39 - DNA, 20 bp, Mus musculus
gccaccgatccacacagagt

SEQ ID NO: 40 - DNA, 20 bp, Drosophila melanogaster
agaaggacgcacaaatctgc

SEQ ID NO: 41 - DNA, 20 bp, Drosophila melanogaster
cgcgtaccttcagcttcttc

SEQ ID NO: 42 - DNA, 19 bp, Drosophila melanogaster
ggcctatcacttcgatgga

SEQ ID NO: 43 - DNA, 21 bp, Drosophila melanogaster
gtctcaatgtgaaccgaatcc

SEQ ID NO: 44 - DNA, 20 bp, Drosophila melanogaster
ccgatgatgacgacgaggat

SEQ ID NO: 45 - DNA, 20 bp, Drosophila melanogaster
gttcgttccgttcgttctcc

SEQ ID NO: 46 - DNA, 18 bp, Drosophila melanogaster
gcccaactgcttgcccaa

SEQ ID NO: 47 - DNA, 20 bp, Drosophila melanogaster
cctcgtaggggtcaatgtcc

SEQ ID NO: 48 - DNA, 22 bp, Drosophila melanogaster
tgaatatctttcgctttgctgg

SEQ ID NO: 49 - DNA, 20 bp, Drosophila melanogaster
agcagcagtattatgatggc

SEQ ID NO: 50 - DNA, 21 bp, Drosophila melanogaster
aagttgctgctctggttgtcg

SEQ ID NO: 51 - DNA, 21 bp, Drosophila melanogaster
gccacacgcagctcattgtag